(19) United States
(12) Patent Application Publication
(10) Pub. No.: US 2026/0088134 A1
(43) Pub. Date: Mar. 26, 2026

(54) Systems and Methods for Spatial Alignment of Cellular Specimens and Applications Thereof

(71) Applicant: The Board of Trustees of the Leland Stanford Junior University, Stanford, CA (US)

(72) Inventors: Aaron M. Newman, San Mateo, CA (US); Milad R. Vahid, Stanford, CA (US); Erin Brown, Stanford, CA (US); Chloe Steen, Oslo (NO)

(73) Assignee: The Board of Trustees of the Leland Stanford Junior University, Stanford, CA (US)

(21) Appl. No.: 18/866,981

(22) PCT Filed: May 18, 2023
(86) PCT No.: PCT/US2023/067198

§ 371 Date: Nov. 18, 2024

Related U.S. Application Data
(60) Provisional application No. 63/364,935, filed on May 18, 2022.

(51) Int. Cl. G16B 45/00; G16B 25/10; G16H 10/40

(52) U.S. Cl. CPC G16B 45/00; G16B 25/10; G16H 10/40

(57) ABSTRACT

Processes to spatially align single cells to yield a specimen map with single cell resolution are provided. Methods can perform spatial omics on a specimen to yield spatial omics data. The spatial omics data can be used in combination with single cell omics data to assign single cells to spatial coordinates to yield a resolved specimen map.

### CROSS REFERENCE TO RELATED APPLICATIONS

This application claims priority to U.S. Provisional Application Ser. No. 63/364,935, entitled Robust alignment of single-cell and spatial transcriptomes with CytoSPACE, filed May 18, 2022, which is incorporated herein by reference in its entirety.

### STATEMENT REGARDING FEDERALLY SPONSORED RESEARCH OR DEVELOPMENT

This invention was made with Government support under contracts CA255450 and CA1871925 awarded by the National Institutes of Health. The Government has certain rights in the invention.

### TECHNICAL FIELD

The disclosure provides description of generating spatially resolved specimen maps at single cell resolution using spatial omics data.

### BACKGROUND

Spatial transcriptomics is a high-throughput methodology of assigning cell types to specific regions within a histological section of tissue or cell culture, as assessed by the collection of transcriptome profiles from that region. Generally, the method independently analyzes very small regions of a histological section of few cells (as few as about five, but typically between 10 and 40 cells) for transcript expression. Transcript expression in individual regions can be assessed in various different methodologies, such as fluorescent in situ hybridization (FISH), in situ sequencing, laser capture microdissection and subsequent transcript analysis, iterative microdigestion and subsequent transcript analysis, and in situ capture and subsequent transcript analysis. The subsequent transcript analysis can be performed using any expression analysis technique, such as quantitative polymerase chain reaction, microarray, and RNA sequencing.

### SUMMARY OF THE INVENTION

Systems and methods of the disclosure render spatially resolved maps of a specimen with single cell resolution. Spatial omics data can be acquired from the specimen. Referential single cell omics data can be utilized to match the spatial omics data. Based on a global optimal solution, single cells derived from the referential single cell omics data can be imputed to a spatial coordinates to yield a spatially resolved map of the specimen.

In some implementations, a method comprises analyzing transcriptomes in a plurality of cells to determine cell type. The method comprises assigning the cells to locations in a tissue sample based on all possible location assignments. The method comprises detecting a genetic and/or spatial signature specific to a condition within the cells assigned to the locations in the tissue sample. The method comprises assaying a sample obtained from a subject to detect the signature. The method comprises reporting presence or severity of the condition in the subject based on the detected signature.

In some implementations, the condition is cancer and the spatial signature predicts a response to therapy, toxicity of a therapy, resistance to a therapy, cancer progression, a likelihood of metastasis, a likelihood of a transition from pre-invasive to invasive cancer, or a likelihood of recurrence.

In some implementations, the method comprises prior to the assigning step, obtaining estimates of fractional abundance of the cell types in the tissue sample and number of cells at the locations.

In some implementations, the genetic and/or spatial signature specific to the condition includes information about proximity or interaction among different types of cells.

In some implementations, the method comprises providing expression profiles for tissue cells at the locations within the tissue sample.

In some implementations, the tissue sample includes a section of a solid tumor.

In some implementations, the assigning step uses a convex optimization function.

In some implementations, the method comprises performing the assaying step for a plurality of test samples each exposed to one of a plurality of candidate compounds and identifying a compound that treats the condition.

In some implementations, the analyzing step includes accessing a database or atlas of the transcriptomes of the cells.

In some implementations, the assignment step ensures a globally optimal assignment of the cells to the locations.

In some implementations, the assigning step uses a shortest augmenting path algorithm.

In some implementations, the condition includes T cell exhaustion.

In some implementations, the analyzing step includes single-cell RNA-sequencing (scRNA-Seq) to obtain the transcriptomes.

In some implementations, a method is for yielding a spatially resolved map of a specimen. The method comprises obtaining, using a computational processing system, spatial omics data from a plurality of regions that cover a specimen. The specimen is a collection of cells that comprises a plurality of cell types. The method comprises estimating, using the computational processing system, a number of cells per region of the plurality of regions from the spatial omics data. The method comprises estimating, using the computational processing system, a fraction of each cell type of the plurality of cell types from the spatial omics data. The method comprises querying, using the computational processing system, referential single cell omics data to match a number of cells for each cell type of the plurality of cell types to yield a set of single cell omics data for spatial assignment. The method comprises, based on a globally optimal solution, assigning, using the computational processing system, single cells from the set of single cell omics data to spatial coordinates to yield a spatially resolved map of the specimen.

In some implementations, a method is for yielding a spatially resolved map of a specimen. The method comprises obtaining, using a computational processing system, spatial omics data from a plurality of regions that cover a specimen. The specimen is a collection of cells that comprises a plurality of cell types. The method comprises estimating, using the computational processing system, a number of cells per region of the plurality of regions. The method comprises, based on a globally optimal solution, concurrently: determining a fraction of each cell type of the plurality of cells; querying, using the computational processing system, single cell omics data to match the number of cells for each cell type of the plurality of cell types to yield a set of single cell omics data for spatial assignment; and assigning, using the computational processing system, single cells from the set of single cell omics data to spatial coordinates to yield a spatially resolved map of the specimen.

In some implementations, a method is for yielding a spatially resolved map of a specimen for a set of one or more cell types. The method comprises obtaining, using a computational processing system, spatial omics data from a plurality of regions that cover a specimen. The specimen is a collection of cells that comprises a plurality of cell types. The method comprises estimating, using the computational processing system, a number of cells per region of the plurality of regions from the spatial omics data. The method comprises estimating, for each region of the plurality regions, using the computational processing system, a fraction of each cell type of the plurality of cell types from the spatial omics data. The method comprises querying, for each region comprising a fraction of a cell type of the set of one or more cell types, using the computational processing system, referential single cell omics data to match the number of cells for each cell type of the plurality of cell types to yield a set of single cell omics data for spatial assignment. The method comprises, based on a globally optimal solution, assigning, for each region comprising a fraction of a cell type of the set of one or more cell types, using the computational processing system, single cells from the set of single cell omics data to spatial coordinates to yield a spatially resolved map of the specimen consisting of the cell types of the set of one or more cell types.

In some implementations, querying the referential single cell omics data to match the number of cells for each cell type of the plurality of cell types further comprises removing, for each region comprising a fraction of a cell type of the set of one or more cell types, using the computational processing system, single cell omics data of one or more single cells within the referential single cell omics data when the number of single cells within the referential single cell omics data is greater than the number of cells estimated within the specimen.

In some implementations, querying the referential single cell omics data to match the number of cells for each cell type of the plurality of cell types further comprises adding, for each region comprising a fraction of a cell type of the set of one or more cell types, using the computational processing system, single cell omics data of one or more single cells within the referential single cell omics data when the number of single cells within the referential single cell omics data is less than the number of cells estimated within the specimen.

In some implementations, each region comprises a number of subregions equal to with the number of cells estimated for each region.

In some implementations, assigning single cells from the set of single cell omics data to spatial coordinates comprises generating, for each region comprising a fraction of a cell type of the set of one or more cell types, using the computational processing system, a matrix of single cell omics profiles with single cells and a matrix of specimen omics profiles with subregions and utilizing, using the computational processing system, the matrices to determine a globally optimal solution.

In some implementations, the method further comprises determining, using the computational processing system, the globally optimal solution by summation of assignments of singles cells to subregions that minimizes a linear cost function.

In some implementations, the spatial omics is one of: spatial transcriptomics, spatial genomics, spatial epigenomics, spatial methylomics, spatial proteomics, or spatial metabolomics.

In some implementations, the method further comprises extracting source material to perform the spatial omics from each region of the plurality of regions. The source material is extracted via laser capture microdissection, iterative microdigestion, or in situ capture.

In some implementations, the spatial omics is spatial transcriptomics. The method further comprises determining expression of a plurality of transcripts via in situ hybridization.

In some implementations, the step of estimating the number of cells per region comprises estimating, using the computational processing system, the number of cells per region of the plurality of regions based on an amount of source material derived from each region, as determined by the spatial omics data.

In some implementations, the step of estimating the number of cells per region comprises estimating, using the computational processing system, the number of cells per region via cell segmentation.

In some implementations, each region of the plurality of regions is examined for segmented nuclei or staining of cell membranes and the estimation of the number of cells is based on the nuclei count or cell membrane count.

In some implementations, estimating a fraction of each cell type of the plurality of cell types is estimated by a deconvolution method.

In some implementations, the deconvolution method is determined from the spatial omics data and an a priori defined reference.

In some implementations, the deconvolution method is determined by: Spatial Seurat, RCTD, SPOTlight, cell2location, or CIBERSORTx.

In some implementations, querying the referential single cell omics data to match the number of cells for each cell type of the plurality of cell types further comprises removing, using the computational processing system, single cell omics data of one or more single cells within the referential single cell omics data when the number of single cells within the referential single cell omics data is greater than the number of cells estimated within the specimen.

In some implementations, querying the referential single cell omics data to match the number of cells for each cell type of the plurality of cell types further comprises adding, using the computational processing system, single cell omics data of one or more single cells within the referential single cell omics data when the number of single cells within the referential single cell omics data is less than the number of cells estimated within the specimen.

In some implementations, adding single cell omics data of one or more single cells comprises duplicating single cell omics data of the single cell omics data.

In some implementations, adding single cell omics data of one or more single cells comprises generating single cell omics data representative of the single cell omics data.

In some implementations, each region comprises a number of subregions equal to with the number of cells estimated for each region.

In some implementations, assigning single cells from the set of single cell omics data to spatial coordinates comprises generating, using the computational processing system, matrix of single cell omics profiles with single cells and a matrix of specimen omics profiles with subregions and utilizing, using the computational processing system, the matrices to determine a globally optimal solution.

In some implementations, the method further comprises determining, using the computational processing system, the globally optimal solution by summation of assignments of singles cells to subregions that minimizes a linear cost function.

In some implementations, the determining the globally optimal solution further comprises solving, using the computational processing system, the globally optimal solution via a shortest augmenting paths-based Jonker-Volgenant algorithm.

In some implementations, the determining the globally optimal solution comprises solving, using the computational processing system, the globally optimal solution via a cost scaling push-relabel method.

In some implementations, the spatially resolved map of the specimen has a single-cell resolution.

In some implementations, a method is for yielding a spatially resolved map of a specimen via spatial transcriptomics. The method comprises obtaining, using a computational processing system, spatial transcriptomics data from a plurality of regions that cover a specimen. The specimen is a collection of cells that comprises a plurality of cell types. The method comprises estimating, using the computational processing system, a number of cells per region of the plurality of regions from the spatial transcriptomics data. The method comprises estimating, using the computational processing system, a fraction of each cell type of the plurality of cell types from the spatial transcriptomics data. The method comprises querying, using the computational processing system, referential single cell transcriptomics data to match the number of cells for each cell type of the plurality of cell types to yield a set of single cell transcriptomics data for spatial assignment. The method comprises, based on a globally optimal solution, assigning, using the computational processing system, single cells from the set of single cell transcriptomics data to spatial coordinates to yield a spatially resolved map of the specimen.

In some implementations, the method comprises extracting RNA from each region of the plurality of regions, wherein the RNA is extracted via in situ capture. The method comprises sequencing the extracted RNA from each region of the plurality of regions to yield the spatial transcriptomics data from the plurality of regions.

In some implementations, the RNA is extracted from each region of the plurality of regions via 10×Genomics Visium or NanoString GeoMX.

In some implementations, the sequencing is performed by one of the following techniques: whole exome sequencing, capture targeted sequencing, amplification-based targeted sequencing, sequencing based on random priming, or end-biased sequencing.

In some implementations, the method comprises determining expression of a plurality of transcripts via in situ hybridization to yield the spatial transcriptomics data from the plurality of regions.

In some implementations, the expression of the plurality of transcripts is determined via Vizgen MERSCOPE, NanoString CosMX, 10×Genomics Xenium, or hybridization-based in situ sequencing.

In some implementations, the step of estimating the number of cells per region comprises estimating, using the computational processing system, the number of cells per region of the plurality of regions based on a number of detectably expressed genes.

In some implementations, the number of detectably expressed genes is determined by a number of unique molecular identifiers.

In some implementations, the step of estimating the number of cells per region comprises estimating, using the computational processing system, the number of cells per region via cell segmentation.

In some implementations, each region of the plurality of regions is examined for segmented nuclei or staining of cell membranes and the estimation of the number of cells is based on the nuclei count or cell membrane count.

In some implementations, estimating a fraction of each cell type of the plurality of cell types is estimated by a deconvolution method.

In some implementations, the deconvolution method is determined from the spatial omics data and an a priori defined reference.

In some implementations, the deconvolution method is determined by: Spatial Seurat, RCTD, SPOTlight, cell2location, or CIBERSORTx.

In some implementations, querying the referential single cell transcriptomics data to match the number of cells for each cell type of the plurality of cell types further comprises removing, using the computational processing system, single cell transcriptomics data of one or more single cells within the referential single cell transcriptomics data when the number of single cells within the referential single cell transcriptomics data is greater than the number of cells estimated within the specimen.

In some implementations, querying the referential single cell transcriptomics data to match the number of cells for each cell type of the plurality of cell types further comprises adding, using the computational processing system, single cell transcriptomics data of one or more single cells within the referential single cell transcriptomics data when the number of single cells within the referential single cell transcriptomics data is less than the number of cells estimated within the specimen.

In some implementations, adding single cell transcriptomics data of one or more single cells comprises duplicating single cell transcriptomics data of the single cell transcriptomics data.

In some implementations, adding single cell transcriptomics data of one or more single cells comprises generating single cell transcriptomics data representative of the single cell transcriptomics data.

In some implementations, each region comprises a number of subregions equal to with the number of cells estimated for each region.

In some implementations, assigning single cells from the set of single cell transcriptomics data to spatial coordinates comprises generating, using the computational processing system, matrix of single cell transcriptomics profiles with single cells and a matrix of specimen transcriptomics profiles with subregions and utilizing, using the computational processing system, the matrices to determine a globally optimal solution.

In some implementations, the method further comprises determining, using the computational processing system, the globally optimal solution by summation of assignments of singles cells to subregions that minimizes a linear cost function.

In some implementations, the determining the globally optimal solution further comprises solving, using the computational processing system, the globally optimal solution via a shortest augmenting paths-based Jonker-Volgenant algorithm.

In some implementations, the determining the globally optimal solution further comprises solving, using the computational processing system, the globally optimal solution via a cost scaling push-relabel method.

In some implementations, the spatially resolved map of the specimen has a single cell resolution.

In some implementations, a method is to diagnose a medical disorder based on spatial signatures. The method comprises rendering a spatially resolved map of a tissue specimen extracted from a patient. The rendering a spatially resolved map comprises generating spatial omics data from a plurality of regions that cover the tissue specimen. The rendering a spatially resolved map comprises querying, using a computational processing system, referential single cell omics data to yield a set of single cell omics data for spatial assignment. The rendering a spatially resolved map comprises, based on a globally optimal solution, assigning, using the computational processing system, single cells from the set of single cell omics data to spatial coordinates to yield the spatially resolved map of the tissue specimen. The method comprises assessing the spatially resolved map to detect a presence of a spatial signature, wherein the spatial signature is associated with a characteristic of a medical disorder. The method comprises determining the patient has the characteristic of the medical disorder by the presence of the spatial signature within the spatially resolved map.

In some implementations, assessing the spatially resolved map to detect the presence of the spatial signature further comprises utilizing the rendered spatially resolved map of the tissue specimen as input in a trained machine learning model to yield a likelihood of the characteristic of medical disorder. Determining the patient has the characteristic of medical disorder is determined by the likelihood of the characteristic of medical disorder.

In some implementations, the characteristic of medical disorder is a response to therapy.

In some implementations, the method further comprises administering the therapy based on a presence of the spatial signature that indicates the patient will respond to the therapy.

In some implementations, the characteristic of medical disorder is a need for a further diagnostic technique to be performed.

In some implementations, the method further comprises performing the further diagnostic technique based on a presence of the spatial signature indicated the patient will need for the further diagnostic technique to be performed.

In some implementations, the method further comprising performing a spatial omics protocol using the tissue specimen extracted from the patient. The spatial omics protocol is utilized to render the spatially resolved map.

In some implementations, the method further comprising extracting the tissue specimen from the patient to perform the spatial omics protocol.

In some implementations, the tissue specimen comprises tissue of a tumor, of a multicellular organ, infiltrated by immune cells, infected with pathogens, interacting with microbiomes.

In some implementations, the medical disorder is cancer, a pathogenic infection, an organ dysfunction, an inflammatory disorder, an autoimmune disorder, diabetes, liver dysfunction, heart disease, or a neurodegenerative disorder.

In some implementations, the characteristic of the medical disorder is a particular pathology, a likelihood of success or failure of a therapy, a severity of the medical disorder, a need for a particular medical intervention, or a likelihood of a future medical complication.

In some implementations, a method is to diagnose a cancer based on spatial signatures. The method comprises rendering a spatially resolved map of a tumor specimen from a patient. Rendering a spatially resolved map comprises generating spatial omics data from a plurality of regions that cover the tumor specimen. Rendering a spatially resolved map comprises querying, using a computational processing system, referential single cell omics data to yield a set of single cell omics data for spatial assignment. Rendering a spatially resolved map comprises, based on a globally optimal solution, assigning, using the computational processing system, single cells from the set of single cell omics data to spatial coordinates to yield the spatially resolved map of the tumor specimen. The method comprises assessing the spatially resolved map to detect a presence of a spatial signature, wherein the spatial signature is associated with a cancer characteristic. The method comprises determining the patient has the cancer characteristic by the presence of the spatial signature within the spatially resolved map.

In some implementations, assessing the spatially resolved map to detect the presence of the spatial signature further comprises utilizing the rendered spatially resolved map of the tumor specimen as input in a trained machine learning model to yield a likelihood of the cancer characteristic. Determining the patient has the cancer characteristic of medical disorder is determined by the likelihood of the cancer characteristic.

In some implementations, the cancer characteristic is a response to a therapy, a toxicity of a therapy, or a resistance to a therapy.

In some implementations, the cancer characteristic is the response to the therapy. The method comprises administering the therapy based on a presence of the spatial signature that indicates the patient will respond to the therapy.

In some implementations, the cancer characteristic is the toxicity of the therapy.

The method further comprises administering the therapy based on a presence of the spatial signature that indicates the therapy is not toxic to the patient.

In some implementations, the cancer characteristic is the resistance to the therapy. The method further comprises administering the therapy based on a presence of the spatial signature that indicates the patient will not be resistant to the therapy.

In some implementations, the therapy comprises one of: immunotherapy, chemotherapy, radiotherapy, a targeted therapy, hormone therapy, or surgical resection.

In some implementations, the method comprises performing a spatial omics protocol using the tumor specimen extracted from the patient. The spatial omics protocol is utilized to render the spatially resolved map.

In some implementations, the method comprises extracting the tumor specimen from the patient to perform the spatial omics protocol.

In some implementations, the cancer characteristic is cancer progression, a likelihood of metastasis, a transition from pre-invasive to invasive cancer, or a likelihood of recurrence.

In some implementations, a method is for training a machine learning model to predict spatial signatures from spatially resolved maps. The method comprises rendering a spatially resolved map of a plurality of multicellular specimens. Each multicellular specimen is associated with a biological characteristic. The method comprises rendering a spatially resolved map of a plurality of multicellular control specimens. each multicellular control specimen is not associated with the biological characteristic Rendering of each spatially resolved map comprises generating spatial omics data from a plurality of regions that cover the specimen. Rendering of each spatially resolved map comprises querying, using a computational processing system, referential single cell omics data to yield a set of single cell omics data for spatial assignment. Rendering of each spatially resolved map comprises, based on a globally optimal solution, assigning, using the computational processing system, single cells from the set of single cell omics data to spatial coordinates to yield the spatially resolved map of the specimen. The method comprises training a machine learning model with each spatially resolved map of the plurality of multicellular specimens and of the plurality of multicellular control specimens to predict the biological characteristic from a spatially resolved map.

In some implementations, the biological characteristic comprises a pathology, a medical disorder, a health status, a metabolic status, an organ status, an activation of multicellular communication, a multicellular transition, or a multicellular response to a stimulus.

In some implementations, each multicellular specimen is a tumor specimen and the biological characteristic is a cancer characteristic selected from: a response to a therapy, a toxicity of a therapy, or a resistance to a therapy.

In some implementations, the therapy comprises one of: immunotherapy, chemotherapy, radiotherapy, a targeted therapy, hormone therapy, or surgical resection.

In some implementations, each multicellular specimen is a tumor specimen and the biological characteristic is a cancer characteristic selected from: cancer progression, a likelihood of metastasis, a transition from pre-invasive to invasive cancer, or a likelihood of recurrence.

In some implementations, the machine learning model is a classifier.

In some implementations, the machine learning model is a regressor.

In some implementations, the machine learning model incorporates a deep neural network (DNN), a convolutional neural network (CNN), a graph neural network (GNN), a recurrent neural network, a long short-term memory (LSTM) network, a kernel ridge regression (KRR), or gradient-boosted random forest decision trees.

In some implementations, the machine learning model incorporates a spatial encoder.

### BRIEF DESCRIPTION OF THE DRAWINGS

The description and claims will be more fully understood with reference to the following figures and data graphs, which are presented as exemplary embodiments of the invention and should not be construed as a complete recitation of the scope of the invention.

FIG. 1 provides an example of a computational method for spatial alignment of cells from spatial omics data.

FIG. 2 provides computing systems for cellular spatial alignment.

FIG. 3A provides a schematic showing CytoSPACE versus conventional methods for decoding the cellular composition of bulk spatial transcriptomic data.

FIG. 3B provides a schematic of a typical CytoSPACE workflow.

FIG. 4A provides a framework for evaluating CytoSPACE using simulated spatial transcriptomic datasets with fully defined single-cell composition and spot resolution.

FIGS. 4B to 4F provide data depicting maintenance of gene-level spatial dependencies in simulated ST data and impact of controlled noise on scRNA-seq query data. 4B: Pearson correlation analysis of log₂ expression levels in (i) scRNA-seq mapped to Slide-seq beads (as part of simulated ST dataset construction) vs. (ii) the original Slide-seq beads. The resulting p-values were Benjamini-Hochberg adjusted separately for each brain region and shown as q-values. *Q<0.05; ***Q<0.001; ****Q<0.0001; ns, not significant. Sub., Subiculum. 4C and 4D: Box plots showing the effect of adding noise to the scRNA-seq query datasets used in simulation experiments. 4E and 4F: UMAPs of scRNA-seq after the addition of noise for mouse cerebellum (4E) and mouse hippocampus (4F) datasets.

FIGS. 5A to 5E provide estimation of alignment uncertainty in simulated ST datasets. 6A: Confidence scores for all mapped cells. 6B: Box plots showing confidence scores stratified by brain region and correct/incorrect assignments. For a given cell of type i, “correct” was defined as spots containing at least one cell of type i. Statistical significance was determined by a two-sided Wilcoxon test. ****P<2e-16. 6C: Box plots showing the area under the curve (AUC) for distinguishing correct from incorrect spots by cell type (n=11, cerebellum; n=17, hippocampus). 6D and 6E: Impact of imposing a 10% confidence score threshold (>0.1) on the fraction (6D) and absolute number (6E) of retained cells. The box center lines, box bounds, and whiskers in panels b and c denote the medians, first and third quartiles and minimum and maximum values, respectively.

FIGS. 6A to 6E provide estimation of cell type fractions and the number of cells per spot in bulk spatial trancriptome data. 6A: Application of Spatial Seurat to infer cell type fractions in simulated ST datasets. Scatter plots show ground truth cell type fractions (x-axis) versus estimated fractions (y-axis) for simulated ST data of mouse cerebellum (top) and hippocampus (bottom) sections with different spot resolutions. Single-cell RNA sequencing data were first perturbed with the addition of noise to 5% of the transcriptome. 6B: Scatter plot showing the number of cells per spot estimated by CytoSPACE in simulated ST datasets (y-axis) versus ground truth (x-axis) at a mean of 5 cells per spot for mouse cerebellum and hippocampus sections. Relative density is depicted by point size. Concordance and significance were assessed by Pearson r or Spearman p and a two-sided t test, respectively. 6C: Same as 6B but showing correlation coefficients (Pearson and Spearman) for all analyzed spot resolutions. All correlations are significant (P<10⁻²⁰). 6D: Paired analysis showing the difference in performance between log₂ adjustment and the non-log linear scale for predicting the number of cells per spot for all six combinations of spot resolutions in simulated ST datasets (mean of 5, 15, and 30) for Pearson and Spearman correlation coefficients. Statistical significance was calculated with a two-sided paired Wilcoxon test. 6E: Concordance between the number of cells per spot imputed by the default RNA-based approach implemented in CytoSPACE (y-axis) and a cell segmentation algorithm (VistoSeg) respectively applied to paired gene expression data and a histological image of an adult mouse brain coronal sample profiled by 10× Visium. The box center lines, box bounds, and whiskers indicate the medians, first and third quartiles and minimum and maximum values within 1.5× the interquartile range of the box limits, respectively. Linear regression, shown with a 95% confidence interval, was applied to the box plot medians. In 6A and 6B, concordance was assessed by Pearson correlation (r), Spearman correlation (p), and/or linear regression (dashed lines). A two-sided t-test was used to assess whether each correlation result was significantly nonzero. No adjustments for multiple comparisons were made.

FIG. 7A provides heat maps depicting CytoSPACE performance for aligning scRNA-seq data (with 5% added noise) to spatial locations in ST datasets simulated with 5 cells per spot, on average.

FIG. 7B provides performance across distinct methods, mouse brain regions, and noise levels for assigning individual cells to the correct spot in simulated ST datasets. Each point represents a single cell type (mouse cerebellum, n=11; mouse hippocampus, n=17). The box center lines, box bounds, and whiskers indicate the medians, first and third quartiles and minimum and maximum values within 1.5× the interquartile range of the box limits, respectively. Statistical significance was assessed relative to CytoSPACE using a two-sided paired Wilcoxon test. The resulting p-values were Benjamini-Hochberg-adjusted for each noise level and tissue type combination and reported as the maximum Q value (*Q<0.05, ***Q<0.001).

FIG. 7C provides extended benchmarking analysis on simulated ST data (related to FIG. 7C). Box plots depicting the fraction of all single-cell transcriptomes assigned to the correct ST spot, shown for different spot resolutions (mean of 5, 15, and 30 cells per spot) and scRNA-seq noise levels (perturbations added to 5%, 10%, and 25% of the transcriptome) for an extended array of 13 methods. Statistical significance was determined using a two-sided paired Wilcoxon test relative to CytoSPACE. P-values were corrected using the Benjamini-Hochberg method and are expressed as q-values (**Q<0.01). The box center lines, box bounds, and whiskers indicate the medians, first and third quartiles and minimum and maximum values within 1.5× the interquartile range of the box limits, respectively.

FIGS. 7D and 7E provide CytoSPACE, Tangram, and CellTrek alignments for all cell types analyzed in simulated ST datasets (related to FIG. 7A). Heat maps depicting single-cell mapping accuracy, defined as the fraction of single cells correctly mapped to their ground truth spot, shown for three methods and all evaluated cell types mapped to mouse cerebellum (n=11 cell types) (7D) and hippocampus (n=17 cell types) (7E) ST datasets defined by simulation, with a mean of 5 cells per spot.

FIGS. 8A to 8C provide performance of CytoSPACE with RCTD. 8A and 8B: Comparison of cell type fractions estimated by Spatial Seurat and RCTD for (8A) simulated datasets with a mean of 5 cells per spot and 5% noise added to scRNA-seq data and (8B) simulated datasets across all analyzed spot resolutions and noise levels. Concordance was assessed by Pearson correlation (r), Spearman correlation (p), and linear regression (dashed lines). A two-sided t-test was used to assess whether each correlation result was significantly nonzero. 8C: Same as FIG. 7C but showing the application of CytoSPACE with RCTD for cell type fraction estimation (rather than Spatial Seurat) against selected comparator methods. The box center lines, box bounds, and whiskers in b and c indicate the medians, first and third quartiles and minimum and maximum values within 1.5× the interquartile range of the box limits, respectively. Statistical significance was determined using a two-sided paired Wilcoxon test relative to CytoSPACE. P-values were corrected using the Benjamini-Hochberg method and are expressed as q-values (**Q<0.01).

FIGS. 9A and 9B provide data showing an association between CytoSPACE performance and inferred global cell type abundance in simulated spatial transcriptomics datasets. 9A: Scatter plots comparing single-cell mapping accuracy in simulated ST datasets (with a mean of 5 cells per spot) with mean cell type fractional abundances inferred by Spatial Seurat for all cell types and noise levels. Linearity was determined by Pearson correlation. 9B: Same as the 9A but summarizing Pearson correlation significance values across all evaluated simulated ST datasets, spot resolutions, and noise levels. The box center lines, box bounds, and whiskers indicate the medians, first and third quartiles and minimum and maximum values within 1.5× the interquartile range of the box limits, respectively. A two-sided t-test was used to assess whether each correlation coefficient was significantly nonzero. P-values were corrected using the Benjamini-Hochberg method and expressed as q-values.

FIGS. 10A to 10E provide data showing an impact of perturbing estimates of cell type fractional abundance and the number of cells per spot. 10A: Box plots showing the effect of perturbation on cell type fractional abundance estimates over five separate trials, expressed relative to the original estimates (left) and in absolute units (right) for mouse cerebellum (top) and hippocampus (bottom) datasets with a mean of 5 cells per spot and 5% noise added to scRNA-seq query datasets. 10B: Box plots showing CytoSPACE performance on simulated ST datasets before and after perturbing cell type fractions for all spot resolutions and scRNA-seq noise levels. 10C: Scatter plot showing the effect of controlled perturbation on the estimated number of cells per spot for a representative simulated ST dataset (mouse hippocampus with a mean of 5 cells per spot). 10D: Box plots showing Pearson correlations between perturbed and original estimates of the number of cells per spot for all evaluated simulated ST datasets across five trials. 10E: Box plots showing CytoSPACE performance on all simulated ST datasets before and after perturbing estimates of the number of cells per spot (related to 10D). P-values were corrected using the Benjamini-Hochberg method and are expressed as q-values (*Q<0.05; **Q<0.01). The box center lines, box bounds, and whiskers in 10A, 10B, 10D, and 10E indicate the medians, first and third quartiles and minimum and maximum values within 1.5× the interquartile range of the box limits, respectively.

FIGS. 11A and 11B provide data showing stability of CytoSPACE cell-to-spot assignments across multiple seeds and distance metrics. 11A: Same as FIG. 7C but showing CytoSPACE performance for 10 different random samplings of each scRNA-seq query dataset. Statistical significance was calculated using a one-way repeated measures ANOVA. ns, not significant. 11B: Same as FIG. 7C but showing CytoSPACE performance on simulated ST datasets using Pearson correlation, Spearman correlation, or Euclidean distance to calculate the CytoSPACE cost matrix. P-values were corrected using the Benjamini-Hochberg method and are expressed as q-values (**Q<0.01).

FIGS. 12A to 12C provide single-cell RNA-seq data mapped onto ST profiles of diverse human tumor specimens. Gray boxes denote cell types without author-supplied annotations in the corresponding scRNA-seq atlas.

FIG. 13A provides workflow for evaluating spatial enrichment in the tumor core or periphery. DEGs, differentially expressed genes.

FIG. 13B provides spatial enrichment of T cell exhaustion genes in T cell transcriptomes mapped by CytoSPACE to a melanoma sample (row 1, panel a). NES, normalized enrichment score.

FIG. 13C provides data same as FIG. 13B but showing NES for 6 scRNA-seq/ST pairs (n=12 values per box) and 3 methods.

FIGS. 14A and 14D provide spatial enrichment of tumor-associated cell states across methods and datasets. 14A: Left: Bubble plot showing the spatial enrichment of exhaustion genes in CD4 and CD8 T cell transcriptomes mapped onto ST spots by CytoSPACE, Tangram, and CellTrek (related to FIG. 13C). Right: Same as FIG. 14B but showing performance for Tangram and CellTrek. Single-cell RNA-seq datasets without annotated plasma cells are indicated by gray boxes (“N/A”). Bubbles denote normalized enrichment scores calculated by pre-ranked GSEA. 14D: Fraction of datasets per cell type for which the expected spatial enrichment direction was correctly inferred by each of the 13 evaluable methods for each of the gene sets analyzed in this work (n=11 distinct gene sets with 12 data points per method, as canonical exhaustion genes were analyzed for CD4 and CD8 T cells). The box center lines, box bounds, and whiskers indicate the medians, first and third quartiles and minimum and maximum values within 1.5× the interquartile range of the box limits, respectively. Statistical significance was determined using a two-sided paired Wilcoxon test relative to CytoSPACE, with p-values corrected using the Benjamini-Hochberg method.

FIG. 14B provides spatial enrichments of CE9 and CE10-specific cell states in data mapped by CytoSPACE and analyzed by pre-ranked GSEA. Datasets without annotations are indicated in gray.

FIG. 14B provides spatial enrichments of CE9 and CE10-specific cell states in data across 13 methods and 66 combinations of dataset pairs and cell states. To unify the expected enrichment direction of cell states, NES values for CE10 were multiplied by −1.

FIGS. 15A to 15F provide data showing robustness of CytoSPACE applied to tumor spatial transcriptomics datasets. 15A: Same as FIG. 9A but analyzing inferred cell type abundances vs. mean CytoSPACE performance across six tumor ST datasets, where performance is defined as cell state enrichments measured by normalized enrichment score (NES). Of note, to unify the expected enrichment directions, NES values for CE10 were multiplied by −1. 15B: Same as FIG. 10A but showing cell type fraction perturbations for a representative CRC ST dataset. 15C: Same as FIGS. 13C and 14C but showing the impact of perturbing cell type fractions on CytoSPACE performance. 15D: Box plots showing Pearson correlations between perturbed and original estimates of the number of cells per spot for all six tumor ST datasets across five trials. 15E: CytoSPACE performance on all six tumor scRNA-seq/ST dataset pairs before and after perturbing estimates of the number of cells per spot across five trials along with “flattening” the number of cells per spot, in which spots were assigned the same number of cells. P-values were corrected using the Benjamini-Hochberg method and expressed as q-values. *Q<0.05; **Q<0.01. 15F: Same as FIGS. 13C and 14C but comparing NES values for cell state enrichment between the default seed and 9 additional random samplings of the scRNA-seq query dataset.

FIGS. 16A and 16B provide single-cell spatial analysis of TREM2⁺ and FOLR2⁺ macrophage states across datasets and methods. 16A: Expected spatial localization of TREM2⁺ and FOLR2⁺ macrophages in human tumors (Nalio Ramos et al.). 16B: Box plots comparing the log₂ fold change of TREM2 and FOLR2 expression in single macrophage/monocyte transcriptomes grouped into ‘near’ (Euclidean distance to tumor=O) and ‘far’ (Euclidean distance to tumor>0) categories. Each point represents an scRNA-seq/ST pair analyzed in FIG. 14B. The box center lines, box bounds, and whiskers denote the medians, first and third quartiles and minimum and maximum values, respectively. Two-group comparisons were performed using a two-sided paired Wilcoxon test (indicated by the horizontal line above each pair of TREM2⁺ and FOLR2⁺ boxes). ns, not significant.

FIGS. 17A and 17B provide UMAP projections of scRNA-seq tumor atlases labeled by predicted spatial locations. UMAP embeddings showing all single-cell transcriptomes mapped by CytoSPACE to ST samples. Cells are colored by lineage (17A) and by relative distance to tumor cells (17B).

FIG. 18A provides a schematic of the mouse nephron and collecting duct system. Known locations of epithelial states are denoted by numbers.

FIG. 18B provides epithelial cell transcriptomes from a mouse kidney scRNA-seq atlas mapped onto a 10× Visium sample of normal mouse kidney by CytoSPACE, shown using jitter within assigned spots.

FIGS. 18C and 18D provide single-cell cartography of the normal mouse kidney using CytoSPACE. 18C: Mouse kidney scRNA-seq atlas mapped onto a 10× Visium sample of normal mouse kidney, shown for epithelial cell transcriptomes mapped by CytoSPACE and colored by the known zonal region of each cell (as in FIG. 18A) superimposed over the Visium histological image. Zone colors of individual epithelial cells mapped by CytoSPACE are averaged per spot. 18D: Scatter plot showing the statistical significance of co-association between podocytes (epithelial state 1) and all other cell types/states mapped by CytoSPACE (x-axis), and the same for parietal cells (epithelial state 2). Spots were scored as ‘present’ if at least one cell of a given cell type was mapped by CytoSPACE, and ‘absent’ otherwise. Significance of co-association was subsequently calculated using a two-sided Fisher's exact test and represented as −log₁₀ p-values. Self-comparisons are denoted by NA (not applicable).

FIGS. 19A to 19C provide epithelial cell transcriptomes from a mouse kidney scRNA-seq atlas mapped onto a 10× Visium sample of normal mouse kidney by CytoSPACE, Tangram, and CellTrek, each cell colored by known distance to the inner medulla.

FIG. 19D provides concordance between predicted and known distances of each epithelial state to the base of the inner medulla.

FIGS. 20A to 20F provide CytoSPACE-guided reconstruction of the nephron and collecting duct system. 20A: Similar to FIG. 18A but showing epithelial cell states colored by physically adjacent phenotypes. The corresponding cell state ontology is provided in Table 5. 20B: UMAP embedding of a normal mouse kidney scRNA-seq atlas (mapped by CytoSPACE) and colored as in 20A. 20C: Left: Heat map showing the pairwise spatial overlap between all kidney epithelial cell states mapped by CytoSPACE to a 10× Visium sample of normal mouse kidney (related to FIG. 19A). Overlap was determined by the Jaccard index and normalized to the maximum value per row. Right: Heat map showing known adjacent states (as in 20A). 20D: Spring layout of the data in 20C, where each cell state is plotted along with its closest 4 neighbors (in rank space) inferred by CytoSPACE. Selected kidney structures are indicated. Edge thickness is proportional to the degree of overlap in rank space. Statistical significance was calculated by a one-sided permutation test. 20E: Scatter plot comparing (i) the distance between each state i and the nth nearest neighbor (state j) predicted by CytoSPACE (median rank across all evaluable states, y-axis) with (ii) the distance between state i and its ground truth nth nearest neighbor (x-axis). Distances between states were calculated as the number of known consecutive states between i and j. Nearest neighbors from 1 to 10 were evaluated. Agreement was assessed by Pearson correlation and Lin's concordance correlation coefficient (CCC). A two-sided t-test was applied to determine if the correlation coefficient was significantly non-zero. 20F: Same analysis as in panel 20E but for all evaluated methods, comparing performance using CCC. DEEPsc assigned all cells to the same spot and was omitted.

FIG. 21A provides Left: MERSCOPE profile of a breast cancer specimen, colored by cell type. Right: scRNA-seq data mapped to the MERSCOPE profile by CytoSPACE, with previously annotated cell types from the scRNA-seq atlas distinguished by color.

FIG. 21B provides enrichment of CD4 T cell states within tumor regions (pre-ranked GSEA), comparing scRNA-seq data mapped to MERSCOPE (CytoSPACE) with MERSCOPE alone.

FIGS. 22A to 22I provide technical assessment of CytoSPACE applied to single-cell ST data. 22A: Workflow for analyses in FIGS. 22B to 22E. 22B: Left: MERSCOPE reference profile of a breast cancer specimen, with major cell types distinguished by color. Right: MERSCOPE query dataset mapped to the reference profile by CytoSPACE, with query cell types distinguished by color. 22C: Concordance of phenotypes between reference and query cells following alignment. 22D: Analysis of mapping accuracy, showing the significance of the Pearson correlation between the log₂ GEPs of (i) the reference cells and (ii) query cells mapped to the reference cells, stratified by cell type. The matrix diagonal captures comparisons between query cell GEPs and their corresponding reference cell assignments. Non-matching pairwise combinations (off-diagonal entries) represent cell-type-specific controls. 22E: Analysis of the retention of pairwise distances between cells after mapping with CytoSPACE. For each cell type, the scatter plot shows a Retention index, defined as the Pearson correlation between matrices Q and R, versus the variance in matrix R (panel a). The significance of the linear regression line was assessed by a two-sided t-test. 22F: Analysis of gene-level concordance, showing the significance of the Pearson correlation between the log₂ expression levels of (i) the scRNA-seq data (Wu et al.) mapped to MERSCOPE and (ii) the original MERSCOPE data, analyzed separately for each gene (n=497 in common) and cell type. As a control, non-matching pairwise combinations of the same 497 genes were also assessed (off-diagonal entries in the correlation matrix). 22G: Concordance of cell type labels between MERSCOPE and scRNA-seq following alignment. 22H: Left: Tumor and adjacent normal regions. Right: FOLR2 expression in single-cell transcriptomes (Wu et al.) annotated as “Macrophages/Monocytes” and mapped by CytoSPACE, showing elevated levels in adjacent normal regions, consistent with expectation. 22I, Same as FIG. 21B but for CD8 T cells. In d and f, a maximum of 1,000 cells and 1,000 off-diagonal correlations per cell type were randomly sampled for analysis. For each cell type, p-values were Benjamini-Hochberg adjusted and expressed as −log₁₀ q-values, which were multiplied by −1 for negative correlations. Group comparisons in d and f were evaluated using a one-sided Wilcoxon test relative to the matrix diagonal and p-values were Benjamini-Hochberg adjusted. ****Q<0.0001. In d and f, the center lines, bounds, and whiskers indicate the medians, first and third quartiles and minimum and maximum values within 1.5× the interquartile range of the box limits, respectively. GEP, gene expression profile.

### DETAILED DESCRIPTION

Turning now to the drawings and data, systems and methods to spatially align cells within a population of cells are provided. In the various embodiments of the systems and methods, spatial omics analysis is performed to assign a cell type to a particular location within a spatially defined population as to map out the cells within that population. The systems and methods can be performed on various multicellular networks that comprise a plurality of cell types within a region of analysis. The systems and methods can determine the spatial relationship between cell types within the region of analysis, providing single cell resolution within the region. The results of the systems and methods can be mapped, annotated, and visualized, resolving the spatial interaction of each cell within the multicellular network assessed.

The various systems and methods can be applied a variety of omics. The term “omics” is to be understood any of a variety of substantially complete cellular analyses. In some implementations, “omics” refers to transcriptomics, genomics, epigenomics, methylomics, proteomics, and metabolomics. Further, as is understood in the field, any and all these omics can be utilized for spatial analysis and thus the systems and methods can be adapted to the specific parameters for performing such analysis. Generally, when any of a particular set of omics can delineate one cell from another cell within a population, the systems and methods as described herein can be applied. For example, genomics can be utilized to differentiate cells within populations of cells with mixed genomics, such as environments of mixed species (e.g., biofilm, microbiomes), and environments of high genomic heterogeneity (e.g., tumors, neural tissue). For more details on spatial transcriptomics, see, e.g., M. Asp, J. Bergenstrahle, and J. Lundeberg, Bioessays. 2020 October; 42(10):e1900221; and P. L. Stahl, et al., Science. 2016 Jul. 1; 353(6294):78-82; the disclosures of which are each incorporated herein by reference. For more details on spatial genomics, see, e.g., T. Zhao, et al., Nature. 2022 January; 601(7891):85-91; and R. U. Sheth, et al., Nat Biotechnol. 2019 August; 37(8):877-883; the disclosures of which are incorporated herein by reference. For more details on spatial epigenomics, see, e.g., T. Lu, et al., Cell. 2022 Nov. 10; 185(23):4448-4464.e17, the disclosure of which is incorporated herein by reference. For more details on spatial methylomics, see, e.g., N. Loyfer, et al., Nature. 2023 January; 613(7943):355-364, the disclosure of which is incorporated herein by reference. For more details on spatial proteomics, see, e.g., E. Lundberg and G. H. H. Borner, Nat Rev Mol Cell Biol. 2019 May; 20(5):285-302, the disclosure of which is incorporated herein by reference. For more details on spatial metabolomics, see, e.g., L. R. Conroy, et al., Nat Commun. 2023 May 13; 14(1):2759, the disclosure of which is incorporated herein by reference. Further, it should be understood that various omics can be combined for spatial analysis, for instance, as described in D. Zhang, et al., Nature. 2023 April; 616(7955):113-122, the disclosure of which is incorporated herein by reference.

The various systems and methods refer to the spatial alignment of cell types. The term “cell type” is to refer to a particular label of a cell that can be differentiated from other cells based on its omics profile. A variety of contributions can affect an omics profile and thus cell type is to be interpreted broadly to potentially include minor variations that are detectable its omics profile. In some instances, a cell type refers to cells having a particular function. For example, cell types can refer to various immune cells (e.g., macrophages, CD4 T-cells, CD 8 T-cells, B-cells, etc.) or to various cells of an organ system (e.g., cardiomyocytes, pericytes, myeloid cells, fibroblasts, adipocytes, endothelial cells, etc.). In some instances, a cell type refers a level of developmental maturation or stemness. For example, cell types can refer to various cells of hematopoietic development (e.g., hematopoietic stem cell, myeloid progenitor, myeloblast, monocyte, macrophage). In some instances, a cell type refers to genetic heterogeneity. For example, cells of a tumor can various amount of somatic mutations that can be differentiated. In some instances, a cell type refers to a cell that has reacted in a particular way to one or more stimuli. For example, a T cell that is naïve to a cancer and a T cell that has infiltrated a cancer. For example, an epithelial cell that has been in contact with a pathogen and an epithelial cell naïve to pathogen contact. In some instances, cell type refers to a variety of species or strains of cells. For example, cells within a microbiome can comprise a variety of types of bacteria. In some instances, a cell type refers to a mixture definitions (e.g., a cell having a particular function, a particular developmental maturation, a particular somatic genetic makeup, and/or a particular response to stimuli).

Spatial omics, especially spatial transcriptomics, has become a powerful tool for delineating spatial differences (e.g., spatial expression patterns) in spatially organized specimens (e.g., primary tissue specimens). Commonly used platforms remain limited to bulk omics measurements, where each spatially-resolved expression profile is derived from a region having as many as 10, 20, or 40 cells or more. To compensate for this, several computational methods have been developed to infer cellular composition in a given bulk omics sample representing a region. Most such methods use reference profiles derived from representative single-cell omics data derived from a particular cell type to deconvolve these into a matrix of cell type proportions (e.g., region comprises X % of cell type 1, Y % of cell type 2, and Z % of cell type 3). These methods lack granularity, hindering the discovery of spatially defined cell states, their interaction patterns, and their surrounding communities.

Alternatively, spatial omics can be performed in situ, meaning the assessment of biomolecules is performed and visualized within the specimen. An advantage of in situ spatial omics is that it provides subcellular resolution. The improved resolution, however, comes with the disadvantage that assessment is limited to a low number of biomolecules (up to about 1000 probes) and lack complex analysis of those molecules (e.g., somatic mutations within genes cannot be assessed). Accordingly, these methods lack the omics depth and complexity that would be desired at single-cell resolution.

To address these limitations, the systems and methods described here were developed to provide single-cell spatial organization. The systems and methods can utilize an efficient computational approach for aligning individual cells from a cell-type reference to precise spatial locations within regions of spatially organized specimens. Unlike other methods, the solution described herein formulates single-cell spatial assignment as a convex optimization problem and solves this problem using a global approach to find an optimum or a minimum error. This systems and methods yield an optimal spatial assignment result and has greater noise tolerance than other common methods. The output is a reconstructed spatial alignment of cells that can be visualized up to single-cell resolution, allowing for better understanding of multicellular ecosystems. For instance, the ecosystems of a tumor microenvironment, a site of immune cell infiltration, various multicellular organ systems, host-pathogen interactions, and microbiomes can be assessed, delineating a spatial organization and communication between various cells.

The systems and methods can spatially align cells utilizing spatial omics and a single-cell reference for cell-types as input. For instance, in the realm of spatial transcriptomics, a set of referential single-cell RNA-seq results classified with a cell type can be utilized. The systems and methods can use the input to determine a fractional abundance of each cell type within the spatial omics sample and a number of cells per spot. In some implementations, fractional abundance can be determined using a deconvolution tool, such as (for example) Spatial Seurat, RCTD, SPOTlight, cell2location, or CIBERSORTx. In some implementations, fractional abundance can be determined iteratively as cells are mapped. In some implementations, the number of cells is inferred by estimating RNA abundance. In some implementations, the number of cells is determined by cell segmentation. The systems and methods further randomly sample the single-cell reference for cell-types to match the predicted number of cells per cell. The systems and methods further assign each cell to spatial coordinates as determined by convex optimization method. In some implementations, the optimization method minimizes a correlation-based cost function constrained by the inferred number of cells per region via a shortest augmenting path optimization algorithm.

The innovative systems and methods described herein transform spatial omics data (at a resolution of about 5 to 20 cells per region) into a spatially arranged map of cells at a single-cell resolution. These systems and methods provide a dramatic improvement to the computational spatial mapping of cells yet to be realized in this technical field. This improvement can be readily appreciated by the results of performing the method, which provide highly accurate single-cell resolution outputs that can be visualized in color-coded maps. The examples described herein compare the innovative methods with the prior state-of-the-art methods and the results of the comparison clearly show the dramatic improvement.

### Spatial Alignment of Specimens

Several embodiments are directed to assign single cells to a spatial alignment from spatial omics data. In many embodiments, spatial omics data is gathered from a plurality of regions and compared with single cell omics data. In some embodiments, a global optimization solution is utilized to align single cells to yield a spatial arrangement.

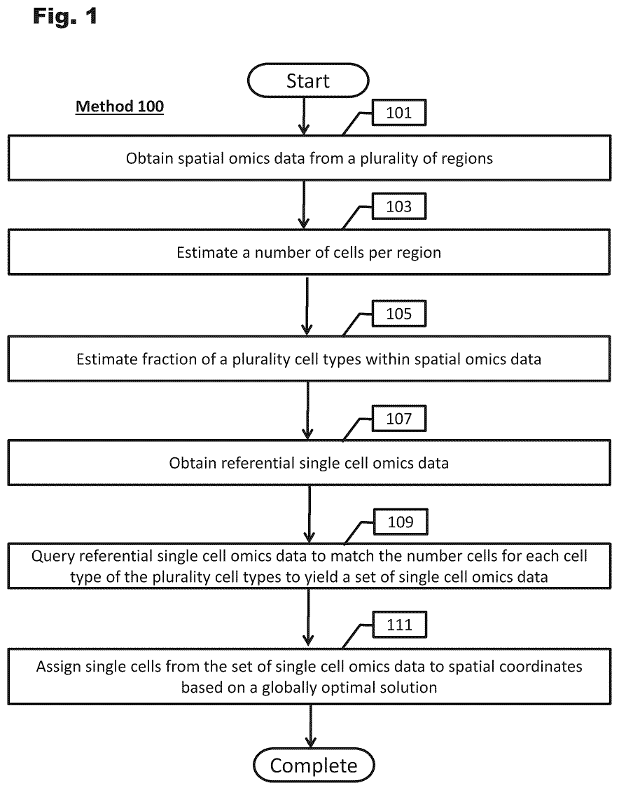

Provided in FIG. 1 is a computational method to yield a spatial arrangement of single cells based spatial omics data. Method 100 can begin by obtaining spatial omics data from a plurality of regions of a specimen. A specimen is a collection of cells having a plurality of cell types that are defined by a spatial arrangement. In some implementations, a specimen is derived from an in vivo source. In some implementations, a specimen is derived from an in vitro source. In some implementations, a specimen is derived from an environmental source. A spatially defined can be a primary tissue specimen, a biofilm or other organized cellular growth, a cell culture, an organoid, or any other specimen that can be defined by a plurality of cell types in a defined spatial arrangement. In various examples, the specimen is a tumor, a multicellular organ specimen, a multicellular organoid specimen, a specimen comprising tissue infiltrated by immune cells, a specimen comprising host tissue and pathogens, or a specimen comprising host tissue and microbiomes. The omics data can be derived from a living specimen or from a fixed specimen, as appropriate to the methodology to perform spatial omics assessment.

Any spatial omics data can be utilized provided it can differentiate the cell types of a specimen. Spatial omics that can be assessed include (but are not limited to) is spatial transcriptomics, spatial genomics, spatial epigenomics, spatial methylomics, spatial proteomics, or spatial metabolomics. As dependent on the omics type, biomolecules can be collected from the cell type and processed for perform the omics analysis.

To perform spatial transcriptomics, RNA can be extracted from the specimen, processed and assessed. Any appropriate method for assessing the transcriptome can be utilized, including (but not limited to) in situ hybridization, in situ sequencing, microarrays, and RNA-sequencing. RNA-sequencing can be whole exome sequencing, capture targeted sequencing, amplification-based targeted sequencing, sequencing based on random priming, or end-biased sequencing, with or without unique molecular identifiers (UMIs). When deciding on how to assess the transcriptome, there is a balance between the depth of genes analyzed and spatial resolution. For instance, in situ methods have subcellular resolution but cannot assess a large depth of genes whereas sequencing methods have low resolution (between about 5 and 20 cells per region) but can provide near-complete transcriptome depth, and. A number of platforms have been developed for performing spatial transcriptomics. Examples for situ hybridization transcriptomics include (but are not limited to) Vizgen MERSCOPE, NanoString CosMX, 10×Genomics Xenium, and hybridization-based in situ sequencing (HybISS) (for more on MERSCOPE, see J. Liu, et al., Life Sci Alliance. 2022 Dec. 16; 6(1):e202201701; for more on CosMX, see S. He, et al., Nat Biotechnol. 2022 December; 40(12):1794-1806; for more on Xenium, see S. M. Salas, et al., bioRxiv 2023.02.13.528102; for more on HybISS, see D. Gyllborg, et al., Nucleic Acids Res. 2020 Nov. 4; 48(19):e112; the disclosure of which are each incorporated herein by reference). Examples for RNA-seq transcriptomics include (but are not limited to) 10×Genomics Visium and NanoString GeoMX, each of which can be combined with high-throughput sequencers (e.g., Illumina HT series) (for more on Visium, see P. L. Stahl, et al., Science. 2016 Jul. 1; 353(6294):78-82; for more on GeoMX, see K. Roberts, bioRxiv 2021.03.20.436265; the disclosures of which are each incorporated herein by reference).

To perform spatial genomics, genomic DNA can be extracted from the specimen, processed and assessed. Any appropriate method for assessing the genome can be utilized, including (but not limited to) microarrays and DNA-sequencing. DNA-sequencing can be whole genome sequencing, whole exome sequencing, capture targeted sequencing, or amplification-based targeted sequencing.

To perform spatial transcriptomics, DNA or RNA can be extracted from the specimen, processed and assessed. Any appropriate method for assessing the epigenome can be utilized, including (but not limited to) chromatin-immunoprecipitation sequencing, chromatin access assessment, and as inferred from RNA-sequencing. Chromatin access assessment can be performed using (for example) assay for transposase-accessible chromatin with sequencing (ATAC-Seq).

To perform spatial methylomics, DNA or RNA can be extracted from the specimen, processed and assessed. Any appropriate method for assessing the methylome can be utilized, including (but not limited to) methylation assessment and as inferred from RNA-sequencing. Methylation assessment can be performed using (for example) bisulfite conversion sequencing or enzymatic methyl sequencing (EM-Seq).

To perform spatial genomics, proteinaceous species can be extracted from the specimen, processed and assessed. Any appropriate method for assessing the proteome can be utilized, including (but not limited to) mass spectrometry and protein microarrays.

To perform spatial metabolomics, metabolites species can be extracted from the specimen, processed and assessed. Any appropriate method for assessing the metabolome can be utilized, including (but not limited to) mass spectrometry and nuclear magnetic resonance spectroscopy.

In many implementations, the source material for performing spatial omics is captured in a plurality of regions. Generally, the plurality of regions covers the specimen to be assessed, or at least a portion thereof. Various methods can be utilized to capture source material from the plurality regions, which may be dependent on various protocols and particular type of omics to be assessed. In some implementations, the source material is extracted from the plurality of regions using laser capture microdissection. In some implementations, the source material is extracted from the plurality of regions using iterative microdigestion. In some implementations, the source material is extracted from the plurality of regions using in situ capture.

In many implementations, spatial omics is performed in situ, meaning the omics analysis is performed directly on an intact specimen. Generally, a fixed specimen (e.g., formalin fixed paraffin embedded tissue) or a fresh frozen specimen is permeabilized and detection of biomolecules for omics analysis is performed therein. Because in situ omics is performed directly on the specimen and provides subcellular resolution, a plurality regions can be defined as desired by the user and can be as granular as a single cell.

Upon assessment of a plurality of regions, spatial omics data can be retrieved. The spatial omics data can be further processed to ensure high data quality for further downstream assessment. For example, reads that map poorly in a sequencing result can be discarded. Many other processing steps can be performed, as is routine when assessing omics data.

Method 100 determines (103) estimates a number of cells per region. The number of cells per region provides an inference of average region size and the number of sub-regions within each region. Several different techniques can be utilized to estimate the number cells per region. In some implementations, the number of cells per region is estimated based on the omics analysis. In some implementations, the number of cells per region is estimated via cell segmentation.

To estimate cell number from omics analysis, an assumption that the source material derived from the plurality of cells can be utilized to infer a cell number. For example, the number of detectably expressed genes per cell corresponds well to the total captured mRNA content, which can be utilized to determine a number of cells. For instance, when single cell RNA seq is performed, the number of detectably expressed genes is utilized to determine when a result has more than a single cell (e.g., result of a doublet). When transcriptomic analysis is performed, the number of unique molecular identifiers provides a proxy for the number of detectably expressed genes and thus can provide an estimate of number of cells per region. Similar analyses can be performed for other omics using inputs of DNA, proteinaceous species, and metabolites.

To estimate cell number via cell segmentation, the regions of specimen are examined for segmented nuclei and/or staining of cell membranes. Based on the nuclei count or cell membrane count, a cell count per region is estimated. Various imaging processing methods can be utilized to perform cell segmentation, such as (for example) VistoSeg and CellPose (M. Tippani, et al., bioRxiv, 2021.2008.2004.452489 (2022); and C. Stringer, et al., Nature Methods 18, 100-106 (2021); the disclosures of which are incorporated herein by reference).

Method 100 estimates (105) a fraction of a plurality of cell types within the spatial omics data. Various techniques can be utilized to estimate cell fraction. In some implementations, cell fraction is estimated by a deconvolution method. In some implementations, cell fraction is computed as part of the optimization solution to assign single cells to spatial coordinates, as discussed in greater detail at step 111.

A number of cellular deconvolution methods to estimate cell fraction for omics data from a plurality of regions are available as computational processing applications. Generally, a global determination of proportional cell types within a specimen are determined from the bulk omics profile using an a priori defined reference (typically derived single cell analysis). Methods for cellular deconvolution that can be utilized include (but are not limited to) Spatial Seurat, RCTD, SPOTlight, cell2location, and CIBERSORTx (for Spatial Seurat, see T. Stuart et al., Cell 177, 1888-1902 e1821 (2019); for RCTD, see D. M Cable, et al., Nature Biotechnology 40, 517-526 (2022); for SPOTlight, see M. Elosua-Bayes, et al., Nucleic Acids Res 49, e50 (2021); for cell2location, see V. Kleshchevnikov, et al., Nature Biotechnology 40, 661-671 (2022); and for CIBERSORTx, see A. M. Newman, Nat Biotechnol 37, 773-782 (2019); the disclosure of which are each incorporated herein by reference).

In some implementations, cellular deconvolution is performed on individual regions (instead of globally) to yield a cell fraction for each region. Regional cellular deconvolution convolution can be performed on each region of the specimen or a specific set of regions. An advantage of performing regional cellular deconvolution is that if a particular cell type (or a set of cell types) are only needed to be assessed for spatial arrangement, the regions that lack the cell type (or set of cell types) can be ignored when assigning cells to spatial coordinates.

Method 100 obtains (107) referential single cell omics data. The single cell omics data is utilized to infer single cell omics data of particular cell types. Referential single cell data can be obtained via a database, published (or otherwise available) data sets, or determined experimentally. To determine experimentally, cells of a particular cell type can be isolated (e.g., via flow cytometry) and their single cell omics data determined.

Method 100 queries (109) the referential single cell omics data to match the number cells for each cell type of the plurality of cell types to yield a set of single cell omics for spatial assignment. This step harmonizes the queried referential single cell omics data with the omics data of the specimen. Harmonization is repeated for each cell type. In some implementations, cell types of the specimen that are lowly represented or unrepresented can be excluded from analysis (e.g., cell type with a fraction below a threshold), as their contribution may not be significant to the final spatial mapping alignment.

If the queried single cell omics data has sequencing data of a number of cells that is greater than the number of cells estimated within the specimen, single cell omics data of one or more single cells is removed such that the single cell omics data matches the number of cells estimated within the specimen. If the queried single cell omics data has sequencing data of a number of cells that is less than the number of cells estimated within the specimen, single cell omics data of one or more single cells is added such that the single cell omics data matches the number of cells estimated within the specimen. Any method of adding single omics data can be utilized. In some implementations, adding single omics data is achieved by duplicating single cell data of the single cell omics data. In some implementations, adding single omics data is achieved by generating single cell data to add to the single cell omics data, which can be generated such that it is representative of the single cell omics data.

Method 100 assigns (111) single cells from the set of single cell omics data to spatial coordinates based on a globally optimal solution. In some implementations, global convex optimization is performed to assign single cells. In some implementations, the optimization is linear. In some implementations, the optimization is nonlinear. To perform optimization, each region can include a set of subregions consistent with the number of cells estimated for each region. A matrix of single cell omics profiles with single cells and a matrix of specimen omics profiles with subregions. The single cells can be assigned to the subregions such that the sum of optimal cell/subregion assignments that provide a global optimization. In some implementations, global optimization is determined by the sum of cell/subregion assignments that minimize a linear cost function.

Various solvers can be utilized to determine a globally optimal solution. In some implementations, the shortest augmenting paths-based Jonker-Volgenant algorithm is utilized determine a globally optimal solution (R. Jonker and A. A. Volgenant, Computing 38, 325-340 (1987), the disclosure of which is incorporated herein by reference). In some implementations, the cost scaling push-relabel method is utilized determine a globally optimal solution (A. V. Goldberg and R. Kennedy, Math. Program. 71, 153-177 (1995), the disclosure of which is incorporated herein by reference).

In some implementations, instead of predetermining a fraction of each cell type of the spatial omics data (as done in step 105), the fraction of each cell type is determined as part of the global optimization. Accordingly, the sum of optimal cell/subregion assignments also assesses variations of cell type number to yield a global optimization.

In some implementations, when regional cellular deconvolution is performed to determine cell fraction in each region, global optimization is only performed on regions containing a set of one or more cell types that are to be assigned coordinates. The other regions can be ignored.

Based upon global optimization of assigning single cells to subregions, a spatially aligned map of the cells can be generated. When global optimization is only performed on regions containing a set of one or more cell types, a focused spatially aligned map of the cells of the set of one or more cell types can be generated. Examples of generated maps are provided within the Examples section below.

While specific examples of methods for yielding a spatial arrangement of single cells using spatial omics data are described above, one of ordinary skill in the art can appreciate that various steps of the process can be performed in different orders and that certain steps may be optional according to some embodiments of the invention. As such, it should be clear that the various steps of the process could be used as appropriate to the requirements of specific applications. Furthermore, any of a variety of processes for yielding a spatial arrangement of single cells using spatial omics data appropriate to the requirements of a given application can be utilized in accordance with various embodiments of the invention.

The spatial alignment of single cells to yield a map of specimen can be utilized in a number of downstream applications. In some implementations, a reconstructed spatial alignment of cells that can be visualized up to single-cell resolution. In some implementations, a spatial alignment of single cells can yield detailed information of an ecosystem of a microenvironment. For instance, assessment of a tumor specimen can provide details of the tumor growth, cancer progression, and/or response to therapy. Any of a number multicellular ecosystems can be assessed, such as (for example) a tumor microenvironment, a site of immune cell infiltration, a multicellular organ system, a host-pathogen interaction, and a microbiome.

Results of a spatial alignment can be utilized to determine various signatures associated with spatial context. For example, when assessing a cancer specimen, signatures associated with therapy response, therapy resistance, cancer progression, and cancer recurrence can be determined. These signatures can then be utilized to formulate diagnostics.

Spatial signatures can be further delineated by training a computational machine learning model to provide a prediction. For example, a plurality tissue samples having an association with a particular biological characteristic can each be assessed for spatial signatures. The particular biological characteristic can be any characteristic, such as a pathology, a medical disorder, a health status, a metabolic status, an organ status, an activation of multicellular communication, a multicellular transition, a multicellular response to a stimulus, or any other characteristic that can be associated with a particular spatial arrangement of cells. In the realm of cancer diagnostics, a cancer characteristic is assessed such as (for example) therapy response, therapy resistance, cancer progression, and cancer recurrence. A machine model can be trained to predict the particular biological characteristic based on a rendered spatially resolved map of single cells. Machine models can inherently detect spatial signatures from the spatially resolved map, even in scenarios in which a trained clinician cannot detect the spatial signature. The model can be trained with multicellular specimens that are known to have an association with the particular characteristic. The model can be further trained with multicellular control specimens that are known to have not be associated with a particular biological characteristics. For example, spatial alignments derived from tumor samples from a plurality of patients that were resistant a particular therapy and spatial alignments derived from tumor samples from a plurality of patients that were responsive a particular therapy can be utilized to train a model to predict a likelihood whether a tumor sample will resist that particular therapy. In various implementations, the training can be supervised, partially supervised, or unsupervised. In some implementations, the machine learning model is a classifier. In some implementations, the machine learning model is a regressor. The model can incorporate one or more of any appropriate architectures, such as (for example) a deep neural network (DNN), a convolutional neural network (CNN), a graph neural network (GNN), a recurrent neural network, a long short-term memory (LSTM) network, a kernel ridge regression (KRR), and gradient-boosted random forest decision trees. In some implementations, the model incorporates a spatial encoder.

Diagnostic procedures can be developed using spatial signatures. These diagnostic procedures can comprise the following steps:
Render a spatially resolved map of a tissue specimen derived from a patient
Assess the spatially resolved map to detect a spatial signature
Based on the spatial signature, determine a diagnosis

In some implementations, a diagnostic procedure can comprise a step that determines a therapy based on the spatial signature. In some implementations, a diagnostic procedure can comprise a step that administers a therapy that is determined based on the spatial signature. In some implementations, a diagnostic procedure can comprise a step that determines a further diagnostic technique to be performed. In some implementations, a diagnostic procedure can comprise a step that performs a further diagnostic technique that is determined based on the spatial signature.

In some implementations, a diagnostic procedure comprises performing a spatial omics protocol using the tissue specimen derived from the patient, where the spatial omics protocol is utilized to develop the spatial alignment. In some implementations, a diagnostic procedure comprises obtaining a tissue specimen from the patient. In some implementations, a diagnostic procedure comprises obtaining a tissue specimen from the patient. Tissue specimens can comprise a tumor, a multicellular organ specimen, a specimen comprising tissue infiltrated by immune cells, a specimen comprising host tissue and pathogens, or a specimen comprising host tissue and microbiomes. In some instances, the patient has disease or a medical disorder and the tissue specimen comprises the disease or a medical disorder or is affected by a medical disorder. Medical disorders can include (but are not limited to) cancer, pathogenic infection, an organ dysfunction, an inflammatory disorder, an autoimmune disorder, diabetes, liver dysfunction, heart disease, or a neurodegenerative disorder. In some implementations, a diagnostic procedure can predict a characteristic of a medical disorder. Characteristics can include (but are not limited to) a particular pathology, likelihood of success or failure of a therapy, a severity of the medical disorder, a need for a particular medical intervention, and a likelihood of a future medical complication.

In the realm of cancer diagnostics, various cancer-related characteristics can be diagnosed. In some implementations, a diagnostic procedure can predict response to a therapy. In some implementations, a diagnostic procedure can predict toxicity of a therapy. In some implementations, a diagnostic procedure can predict resistance to a therapy. Therapies can include (but are not limited to) immunotherapy, chemotherapy, radiotherapy, a targeted therapy, hormone therapy, and surgical resection. In some implementations, a diagnostic procedure can predict cancer progression. In some implementations, a diagnostic procedure can predict a likelihood of metastasis. In some implementations, a diagnostic procedure can predict a likelihood of a transition from pre-invasive to invasive cancer. In some implementations, a diagnostic procedure can predict a likelihood of recurrence.

### Systems of Spatial Alignment

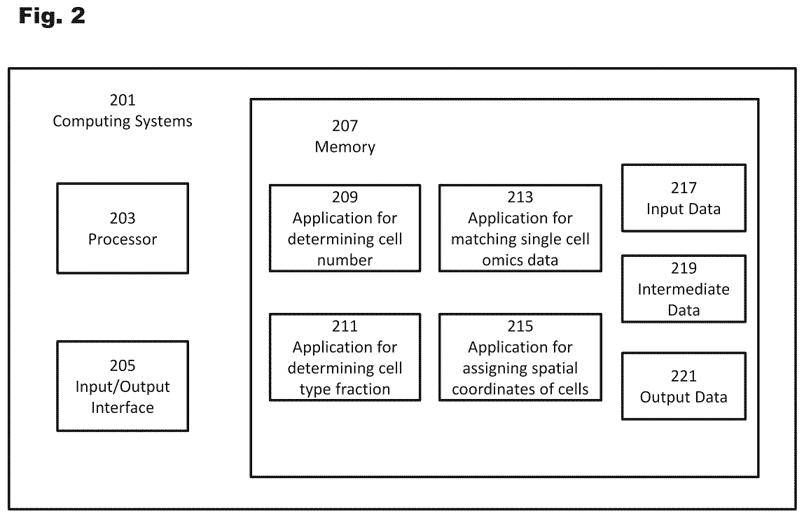

Turning now to FIG. 2, a computational processing system for cellular spatial alignment in accordance with various embodiments of the disclosure typically utilizes a processing system including one or more of a CPU, GPU and/or neural processing engine. In a number of embodiments, spatial omics input data is processed to spatially align cells using single cell omics data via a computational processing system. In some embodiments, the computational processing system is housed within a computing device that is in direct association a system for capturing spatial omics data. In some embodiments, the computational processing system is housed separately from and receives the acquired spatial-omics data. In certain embodiments, the computational processing system is in communication with the system for capturing spatial-omics data. In various embodiments, the processing system communicates with the system for capturing spatial omics data by any appropriate means (e.g., a wireless connection). In certain embodiments, the computational processing system is implemented as a software application on a computing device such as (but not limited to) remote processor, CPU, mobile phone, a tablet computer, and/or portable computer.

A computational processing system in accordance with various embodiments of the disclosure is illustrated in FIG. 2. The computational processing system 201 includes a processor system 203, an I/O interface 205, and a memory system 207. As can readily be appreciated, the processor system 203, I/O interface 205, and memory system 207 can be implemented using any of a variety of components appropriate to the requirements of specific applications including (but not limited to) CPUs, GPUs, ISPs, DSPs, wireless modems (e.g., WiFi, Bluetooth modems), serial interfaces, volatile memory (e.g., DRAM) and/or non-volatile memory (e.g., SRAM, and/or NAND Flash). In the illustrated embodiment, the memory system is capable of storing a number of applications and/or data. Applications can include (but is not limited to) an application for determining cell number 209 (e.g., number cells in a spot), an application for determining cell type fraction 211 (e.g., cell types within a spot), an application for matching single cell omics data 213 (e.g., using single cell sequencing data reference to match cell types to a spot), and an application for assigning spatial coordinates of cells (e.g., assignment of cells to particular spots). The various applications can be downloaded and/or stored in non-volatile memory. When executed, the various applications are each capable of configuring the processing system to implement computational processes including (but not limited to) the computational methods described above and/or combinations and/or modified versions of the computational methods described above. In several embodiments, the various applications utilize input data 217, generate and/or utilize intermediate data 219, and generate output data 221, each of which can be stored in the memory system, which can be stored transiently for performing the computational methods or for longer terms such that the data can be retrieved at a later time point. Input data can include (but are not limited to) spatial-omics data and single cell sequencing data. Intermediate data can include (but are not limited to) cell number per spot, cell type fraction, and a likelihood that a single cell sequencing result matches spatial-omics data. Output data ca include (but is not limited to) assignment of cells to spatial coordinates and visualization of the spatial alignment of cells. It is to be understood that input data 217, intermediate data 219, and output data 221 can be utilized in number of different ways and thus should not be limited in any particular way. For instance, any data can be utilized as an output to an output interface (e.g., monitor or other computational system) or utilized as an input for any other process.

While specific computational processing systems are described above with reference to FIG. 2, it should be readily appreciated that computational processes and/or other processes utilized in the provision of spatial cell alignment with various embodiments of the disclosure can be implemented on any of a variety of processing devices including combinations of processing devices. Accordingly, computational devices in accordance with embodiments of the disclosure should be understood as not limited to specific computational processing systems and/or cellular spatial alignment applications. Computational devices can be implemented using any of the combinations of systems described herein and/or modified versions of the systems described herein to perform the processes, combinations of processes, and/or modified versions of the processes described herein.

### Examples

The embodiments of the disclosure will be better understood with the several examples provided within. Many exemplary results of methods to yield a spatial alignment of individual cells from scRNA-seq are described. As can be readily discerned from one particular implementation, CytoSPACE, the methods as described herein outperform several other methods currently in practice.

High-Resolution Alignment of Single-Cell and Spatial Transcriptomes with CytoSPACE

Single-cell spatial organization is a key determinant of cell state and function. For example, in human tumors, local signaling networks differentially impact individual cells and their surrounding microenvironments, with implications for tumor growth, progression, and response to therapy. While spatial transcriptomics (ST) has become a powerful tool for delineating spatial gene expression in primary tissue specimens, commonly used platforms, such as 10× Visium, remain limited to bulk gene expression measurements, where each spatially-resolved expression profile is derived from as many as 10 cells or more (J. Hu, et al., Comput Struct Biotechnol J 19, 3829-3841 (2021), the disclosure of which is incorporated herein by reference).

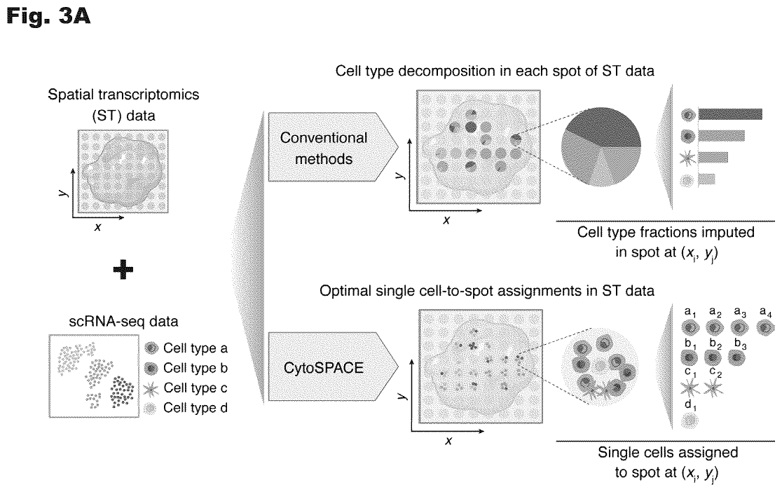

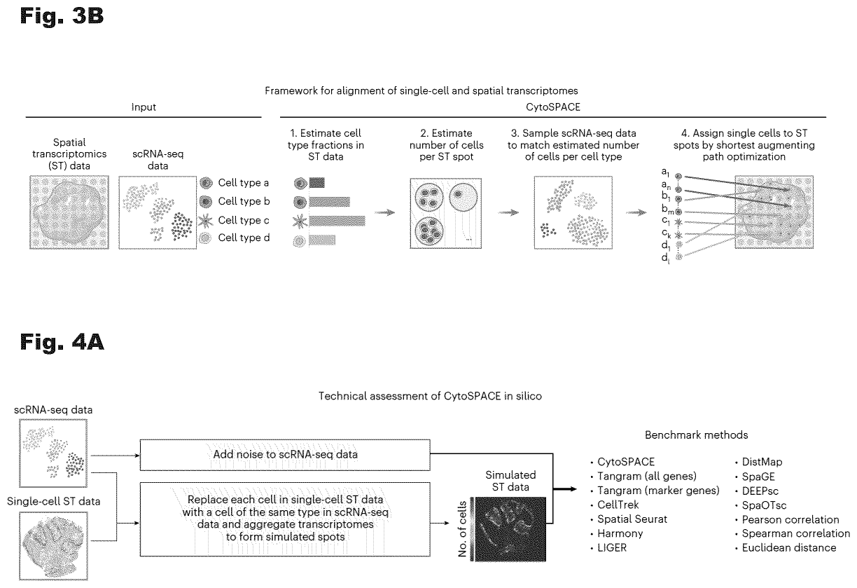

Accordingly, several computational methods have been developed to infer cellular composition in a given bulk ST sample. Most such methods use reference profiles derived from single-cell RNA sequencing (scRNA-seq) data to deconvolve ST spots into a matrix of cell type proportions. However, these methods lack single-cell resolution, hindering the discovery of spatially defined cell states, their interaction patterns, and their surrounding communities (FIG. 3A).

To address this challenge, cellular (Cyto) Spatial Positioning Analysis via Constrained Expression alignment (CytoSPACE) was developed as an example of providing single-cell spatial organization. CytoSPACE is an efficient computational approach for mapping individual cells from a reference scRNA-seq atlas to precise spatial locations in a bulk or single-cell ST dataset (FIGS. 3A and 3B). Unlike other methods (see T. Biancalani et al., Nature Methods 18, 1352-1362 (2021); and R. Wei Nature Biotechnology 40, 1190-1199 (2022)), the solution described herein formulates single-cell/spot assignment as a convex optimization problem and solves this problem using the Jonker-Volgenant shortest augmenting path algorithm (R. Jonker and A. A. Volgenant, Computing 38, 325-340 (1987), the disclosure of which is incorporated herein by reference). This approach guarantees an optimal mapping result while exhibiting improved noise tolerance. The output is a reconstructed tissue specimen with both high gene coverage and spatially resolved scRNA-seq data suitable for downstream analysis, including the discovery of context-dependent cell states. On both simulated and real ST datasets, it was found that CytoSPACE substantially outperforms related methods for resolving single-cell spatial composition.

CytoSPACE proceeds in four main steps (FIG. 3B). First, to account for the disparity between scRNA-seq and ST data in the number of cells per cell type, two parameters are required: (i) the fractional abundance of each cell type within the ST sample and (ii) the number of cells per spot. The fractional abundance is determined using an external deconvolution tool, such as Spatial Seurat, RCTD, SPOTlight, cell2location, or CIBERSORTx (for Spatial Seurat, see T. Stuart et al., Cell 177, 1888-1902 e1821 (2019); for RCTD, see D. M Cable, et al., Nature Biotechnology 40, 517-526 (2022); for SPOTlight, see M. Elosua-Bayes, et al., Nucleic Acids Res 49, e50 (2021); for cell2location, see V. Kleshchevnikov, et al., Nature Biotechnology 40, 661-671 (2022); and for CIBERSORTx, see A. M. Newman, Nat Biotechnol 37, 773-782 (2019); the disclosure of which are each incorporated herein by reference). By default, the number of cells is directly inferred by CytoSPACE using an approach for estimating RNA abundance, though alternative methods including cell segmentation approaches can also be used (see, e.g., M. Tippani, et al., bioRxiv, 2021.2008.2004.452489 (2022); and C. Stringer, et al., Nature Methods 18, 100-106 (2021); the disclosures of which are each incorporated by reference). Once both parameters are estimated, the scRNA-seq dataset is randomly sampled to match the predicted number of cells per cell type in the ST dataset. Up-sampling is done for cell types with insufficient representation, either by drawing with replacement or by introducing placeholder cells. Finally, CytoSPACE assigns each cell to spatial coordinates in a manner that minimizes a correlation-based cost function constrained by the inferred number of cells per spot via a shortest augmenting path optimization algorithm. An efficient integer programming approximation method that yields comparable results is also provided (A. V. Goldberg and R. Kennedy, Math. Program. 71, 153-177 (1995), the disclosure of which is incorporated herein by reference).

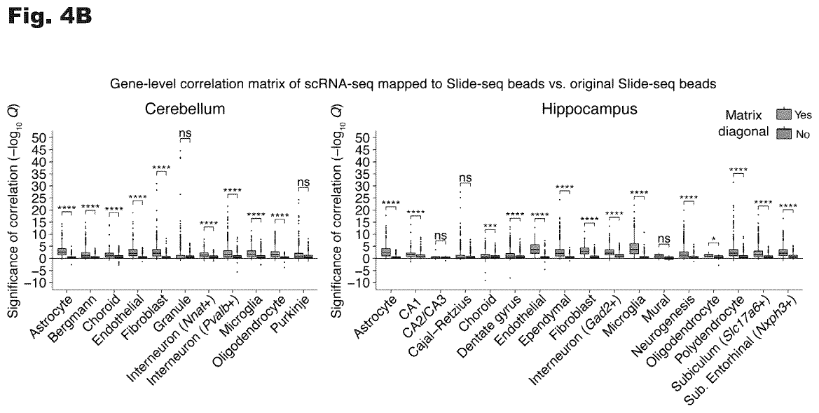

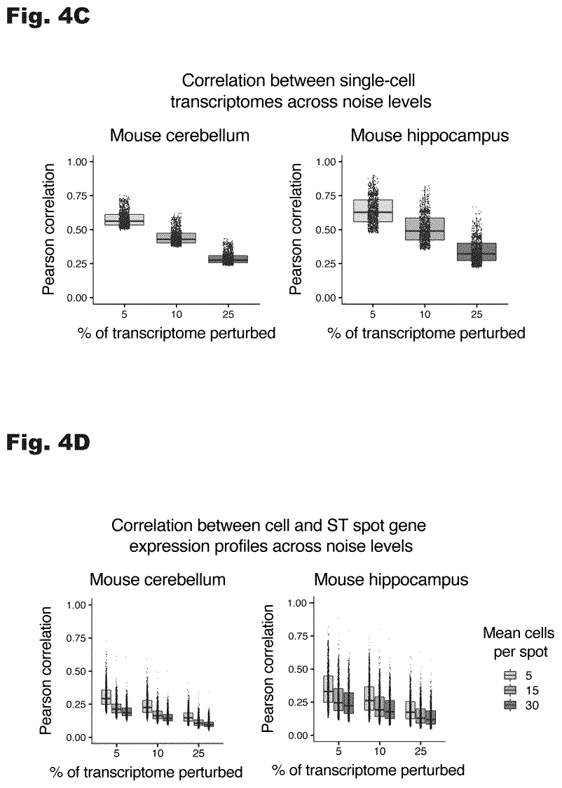

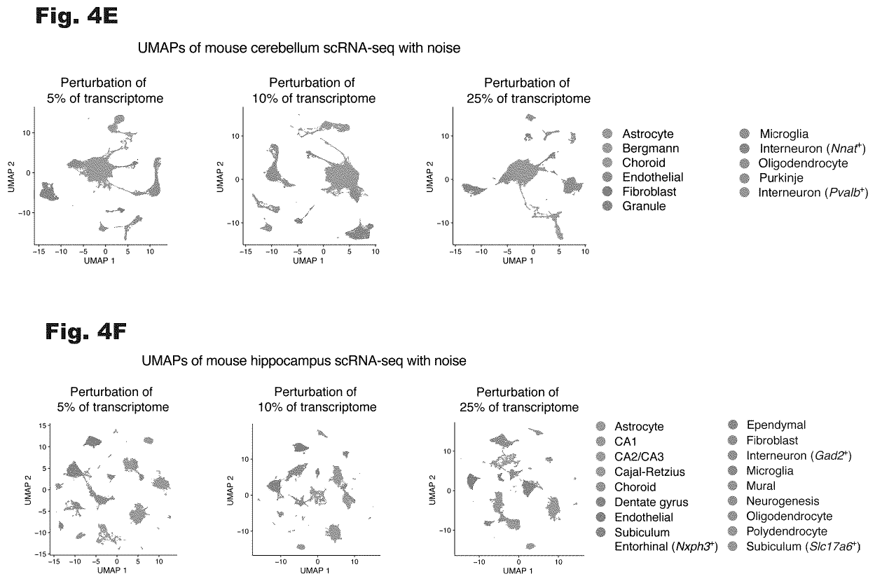

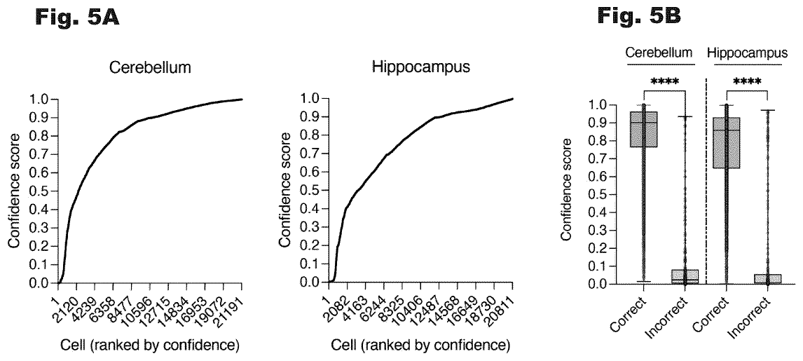

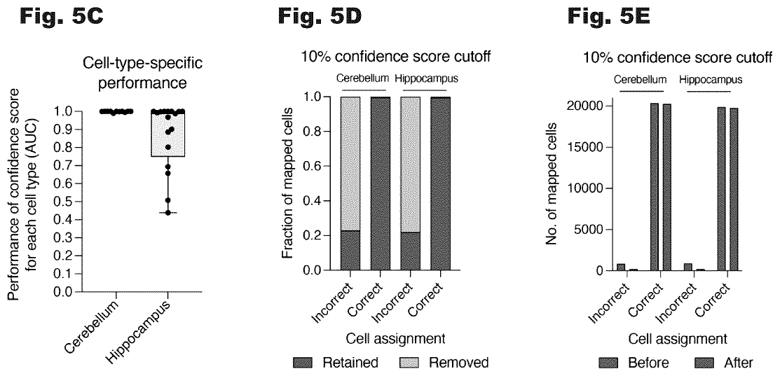

To test the performance of CytoSPACE, ST datasets were simulated with fully defined single-cell composition. For this purpose, previously published mouse cerebellum (n=11 major cell types) and hippocampus (n=17 major cell types) data we leveraged. The data were generated using Slide-seq, a platform with high spatial resolution (approximately single cell) but limited gene coverage (FIG. 4A) (for more on Slide-seq, see S. G. Rodriques, et al., Science 363, 1463-1467 (2019), the disclosure of which is incorporated herein by reference). To increase transcriptome representation while maintaining spatial dependencies, each Slide-seq bead was replaced with the most correlated single-cell expression profile of the same cell type derived from an scRNA-seq atlas of the same brain region (FIG. 4B) (for more on the atlas, see A. Saunders, Cell 174, 1015-1030 e1016 (2018), the disclosure of which is incorporated herein by reference). Then, a spatial grid with tunable dimensions was superimposed in order to pool single-cell transcriptomes into pseudo-bulk transcriptomes. This was done across a range of realistic spot resolutions (mean of 5, 15, and 30 cells per spot). To guarantee a unique spatial address for every cell in the scRNA-seq query dataset, a paired scRNA-seq atlas was created from the cells underlying each pseudo-bulk ST array. Finally, to emulate technical and platform-specific variation between scRNA-seq and ST datasets, noise in varying amounts was added to the scRNA-seq data (FIG. 4C to 4F). Collectively, these datasets allow rigorous assessment of cell-to-spot alignment, including orthogonal approaches for studying alignment quality (FIG. 5A to 5E).

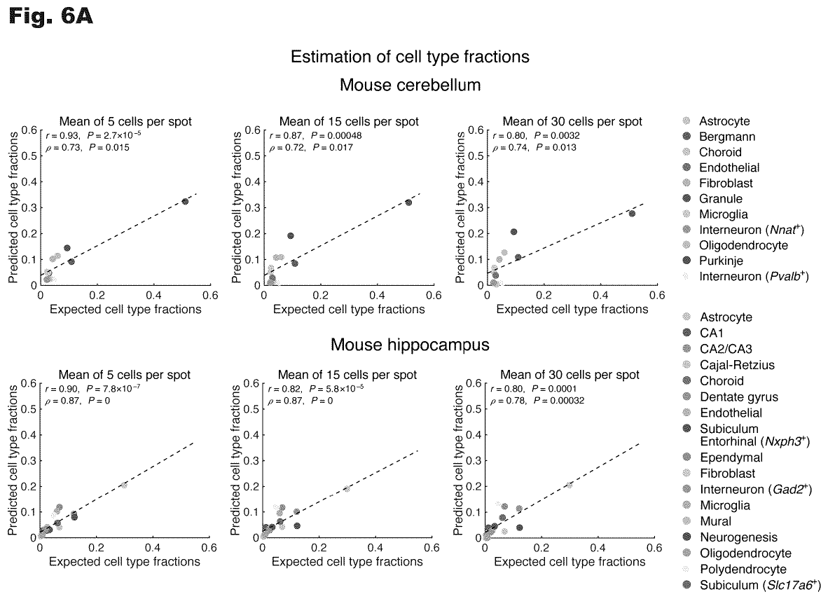

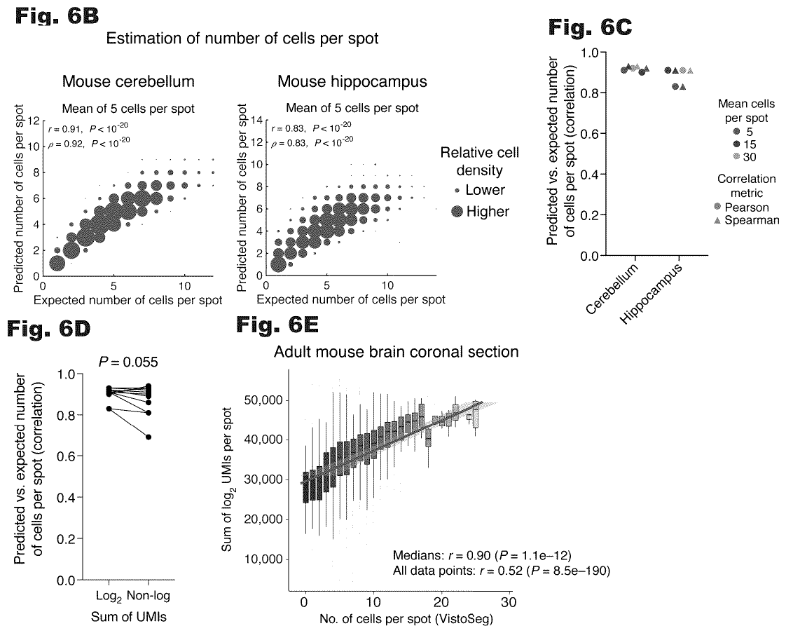

Next, methods for CytoSPACE parameter inference were evaluated. For cell type enumeration, Spatial Seurat was employed, which showed strong concordance with known global proportions in simulated ST datasets (FIG. 6A). To approximate the number of cells per spot, a simple approach was implemented based on RNA abundance estimation. This approach was correlated with ground truth expectations in simulated ST data and cell segmentation analysis of the matching histological image from real ST data (FIGS. 6B to 6E).

CytoSPACE was benchmarked against 12 previous methods, including two recently described algorithms for scRNA-seq and ST alignment: Tangram, which integrates scRNA-seq and ST data via maximization of a spatial correlation function using nonconvex optimization; and CellTrek, which uses Spatial Seurat to identify a shared embedding between scRNA-seq and ST data and then applies random forest modeling to predict spatial coordinates. A few naïve approaches were also assessed, including Pearson correlation and Euclidean distance. To compare outputs, each cell was assigned to the spot with the highest score (all approaches but CellTrek) or the spot with the closest Euclidean distance to the cell's predicted spatial location (CellTrek only).

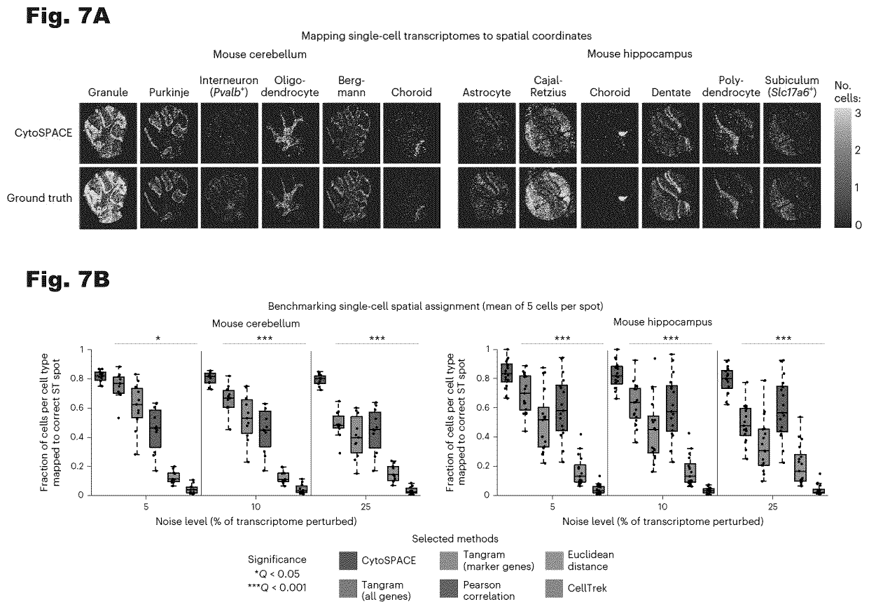

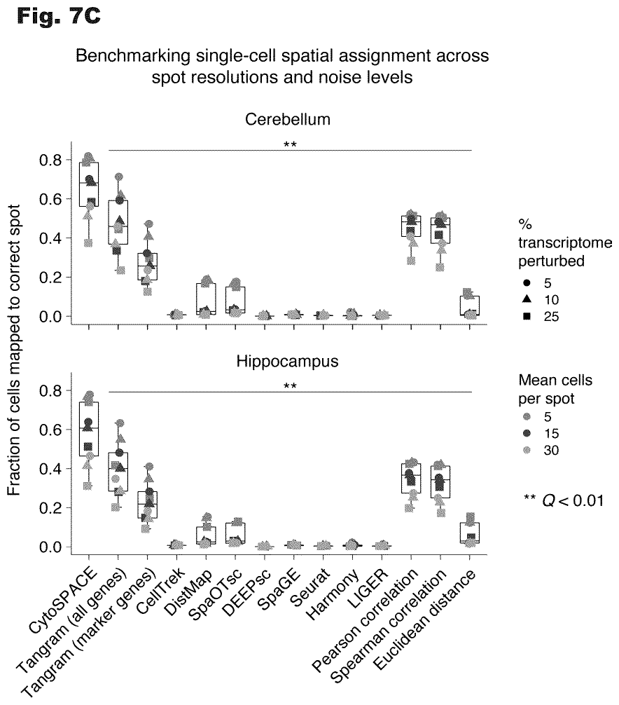

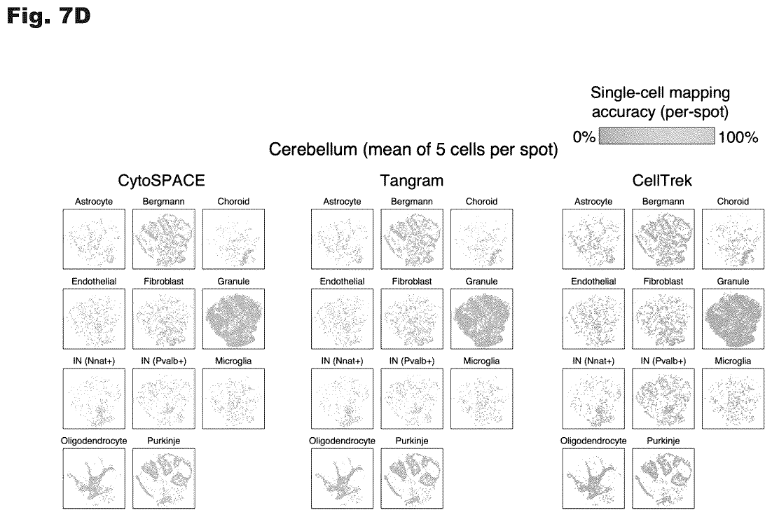

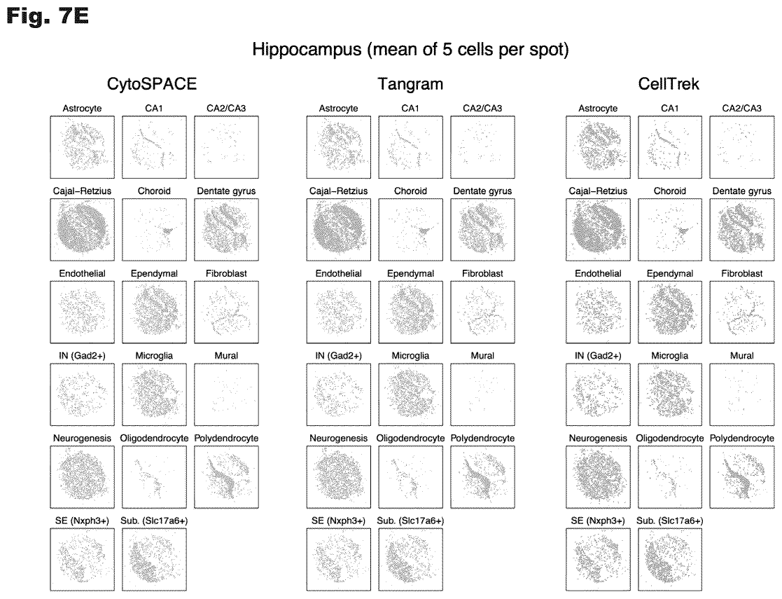

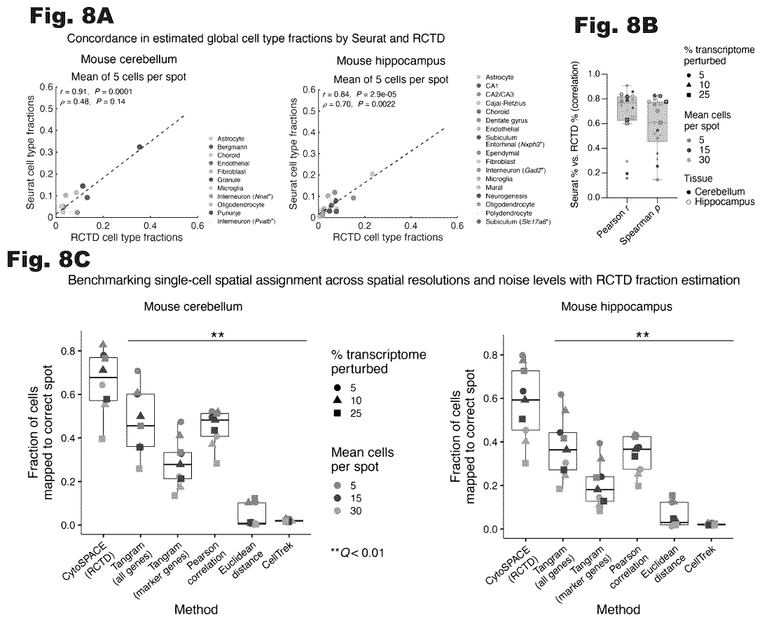

Across multiple evaluated noise levels and cell types, CytoSPACE achieved substantially higher precision than other methods for mapping single cells to their known locations in simulated ST datasets (FIGS. 7A to 7E and Table 1). This was true for multiple spatial resolutions independent of brain region, both for individual cell types and across all evaluable cells (FIGS. 7B and 7C). We also obtained similar results with an independent method for determining cell type abundance in ST data (RCTD) (FIGS. 8A to 8C).

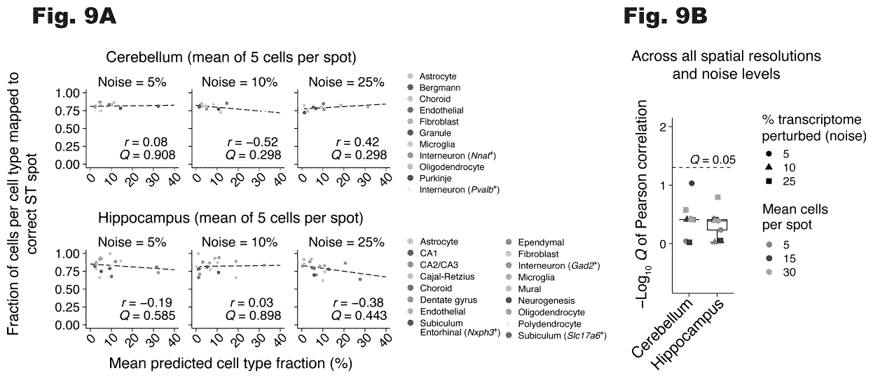

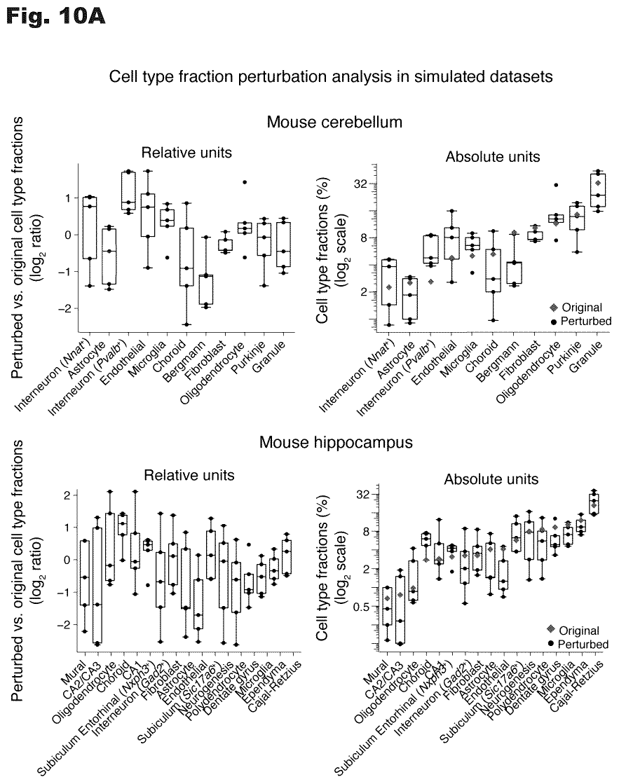

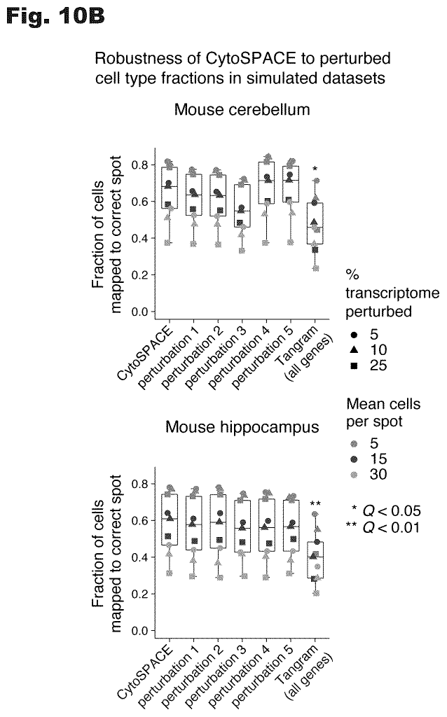

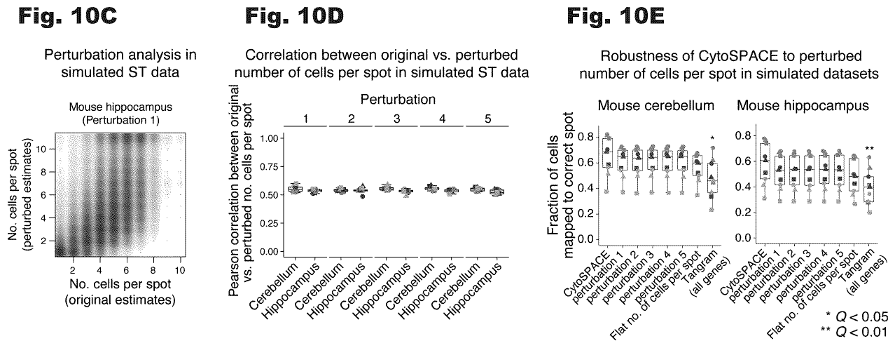

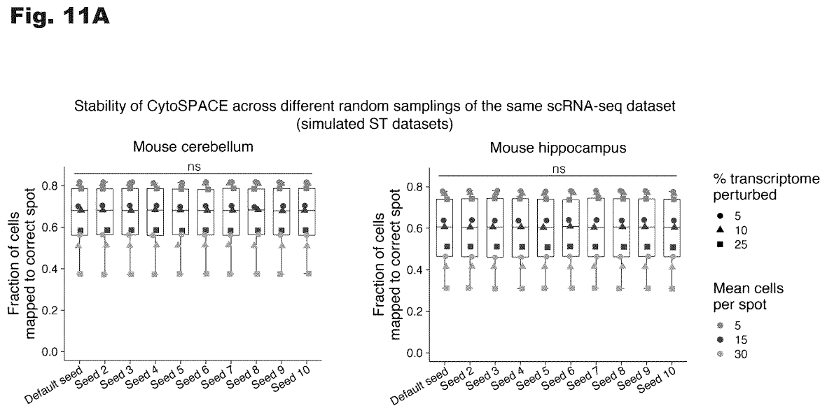

The robustness of CytoSPACE to variation in key input parameters was assessed (steps 1-3 in FIG. 3B). First, estimated cell type abundance was considered, which ranged from a mean of 0.025% to 32% in simulated ST datasets (FIGS. 9A and 9B). Despite this range, no significant correlation with mapping precision was observed ((FIGS. 9A and 9B). Next, experiments were performed in which estimates of (i) cell type abundance and (ii) the number of cells per spot were systematically perturbed. In all cases, CytoSPACE continued to outperform previous methods (FIGS. 10A to 10E). Lastly, output stability when sampling the scRNA-seq query dataset with different seeds was tested (step 3 in FIG. 3B) and when using different distance metrics to calculate the CytoSPACE cost function. Across multiple runs and distance metrics, results remained consistent (FIGS. 11A and 11B). Collectively, these data highlight the robustness of CytoSPACE and underscore its potential to deliver improved spatial mapping of scRNA-seq data.

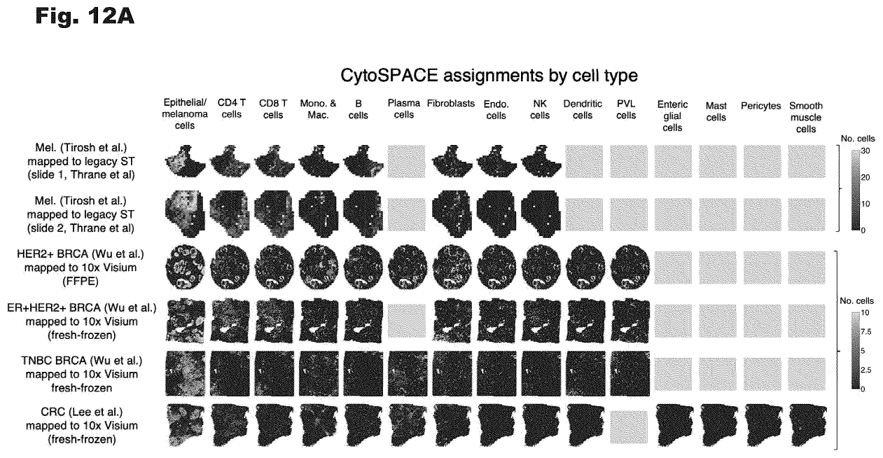

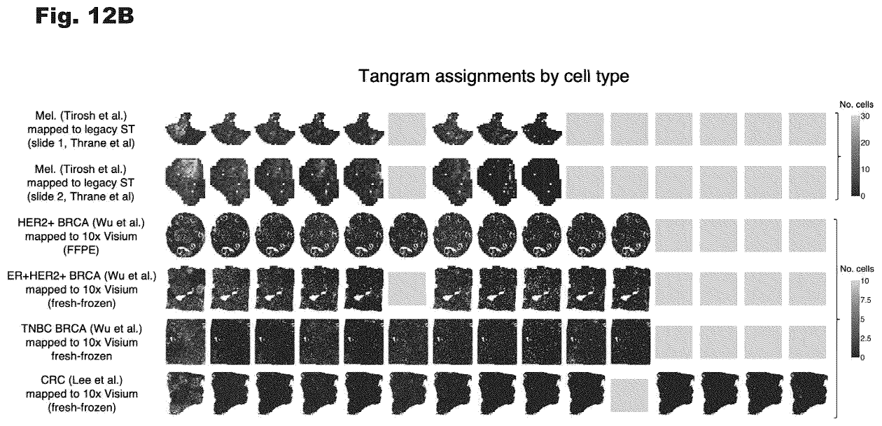

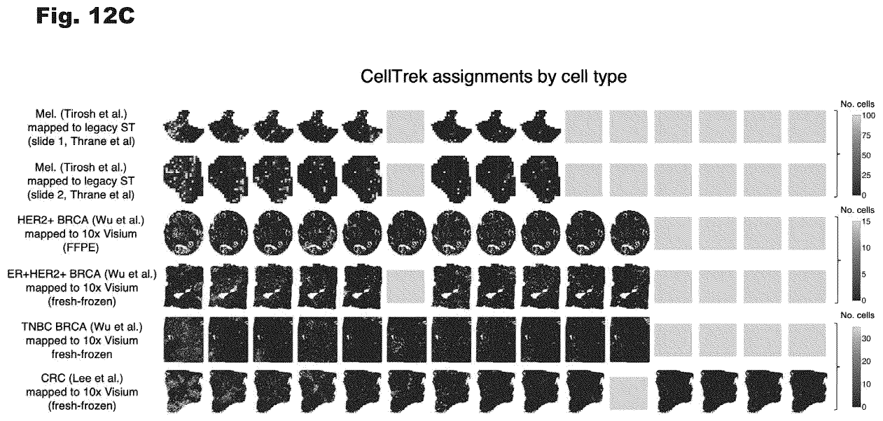

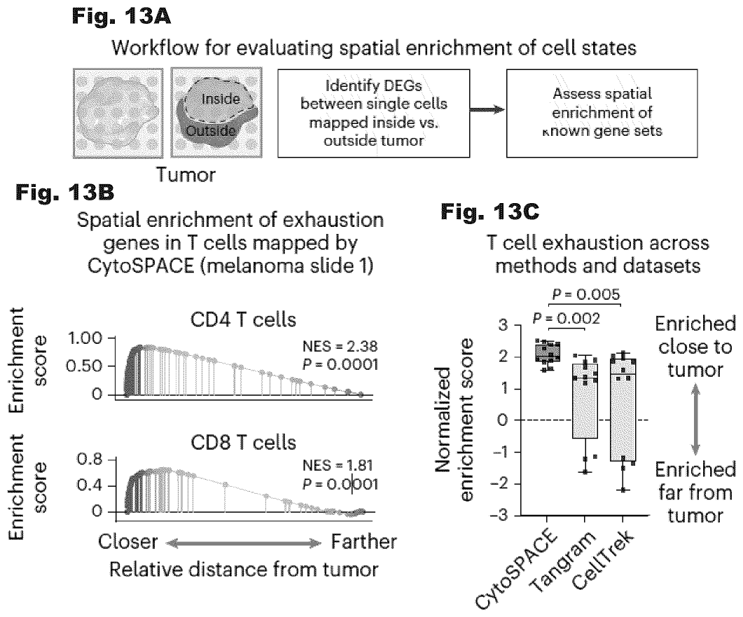

To evaluate performance on real ST datasets, primary tumor specimens were examined. The primary tumor specimens were from three types of solid malignancy: melanoma, breast cancer, and colon cancer. In total, six scRNA-seq/ST combinations, encompassing six bulk ST samples (n=4 Visium; n=2 legacy ST), including one HER2⁺ formalin fixed paraffin embedded (FFPE) breast tumor specimen and three scRNA-seq datasets from matching tumor subtypes, were analyzed (Table 2). All cell types in each scRNA-seq dataset were aligned by CytoSPACE and compared to Tangram and CellTrek (FIGS. 12A to 12C). CytoSPACE was highly efficient, processing a Visium-scale dataset in approximately 5 minutes on a single CPU core (Table 3). This was true regardless of whether shortest augmenting path or integer programming approximation approaches were applied, both of which achieved comparable results (Table 4). To quantitatively compare the recovery of cell states with respect to spatial localization patterns in the tumor microenvironment (TME), assigned cells were dichotomized into two groups within each cell type by their proximity to tumor cells. It was then assessed whether gene sets marking TME cell states with known localization were skewed in the expected orientation (FIG. 13A).

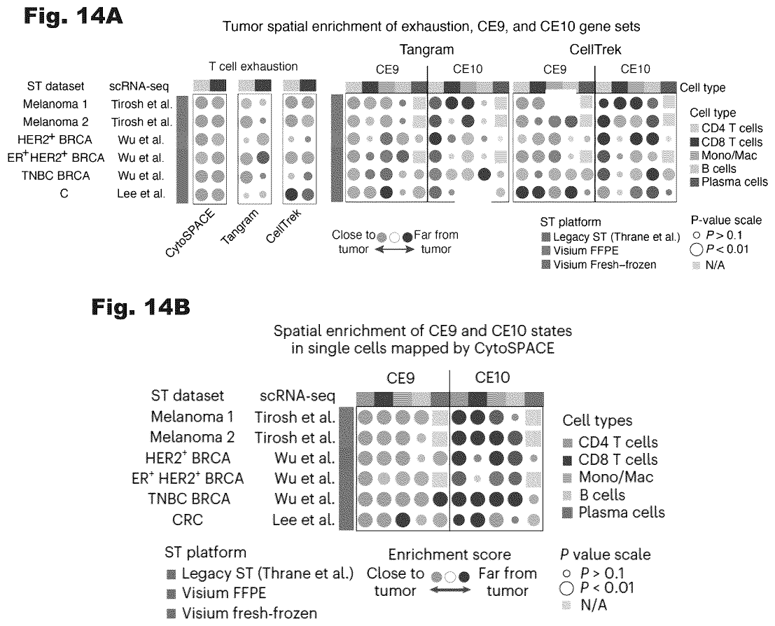

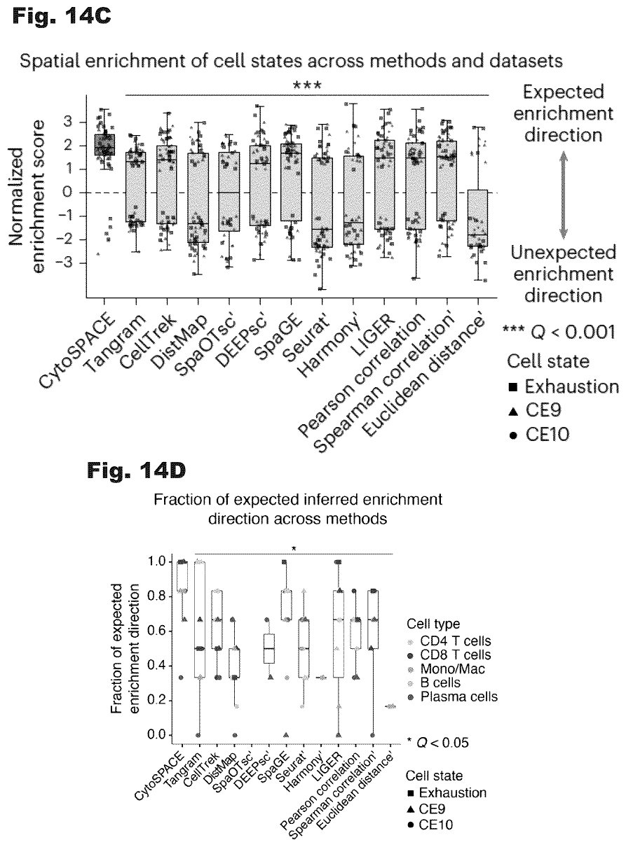

T cell exhaustion, a canonical state of dysfunction arising from prolonged antigen exposure in tumor-infiltrating T cells, was first considered. Consistent with expectation, CytoSPACE recovered spatial enrichment of T cell exhaustion genes in CD4 and CD8 T cells mapped closest to cancer cells in all six scRNA-seq and ST dataset combinations (FIGS. 13B, 13C and 14A). In contrast, Tangram and CellTrek produced single-cell mappings with substantially lower enrichment of T cell exhaustion genes in the expected orientation, with 25% to 33% of cases showing enrichment in the opposite direction, away from the tumor core (FIGS. 13C and 14A).

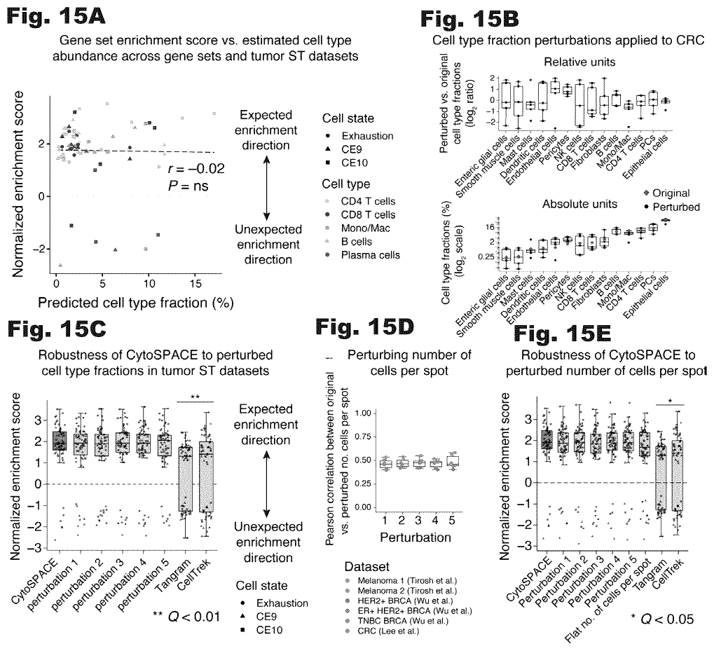

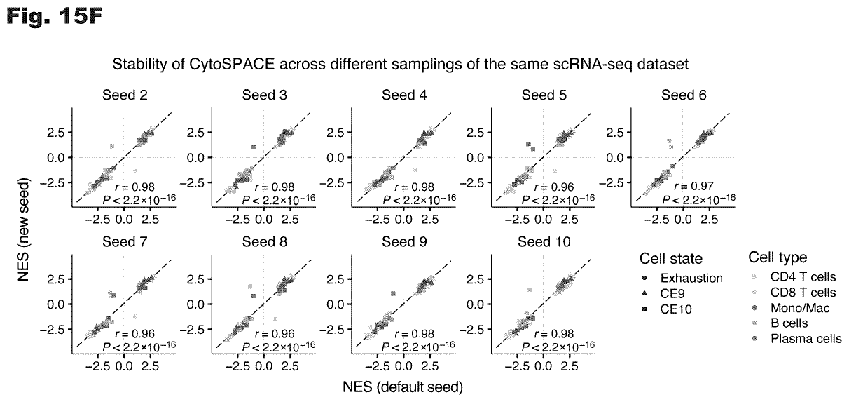

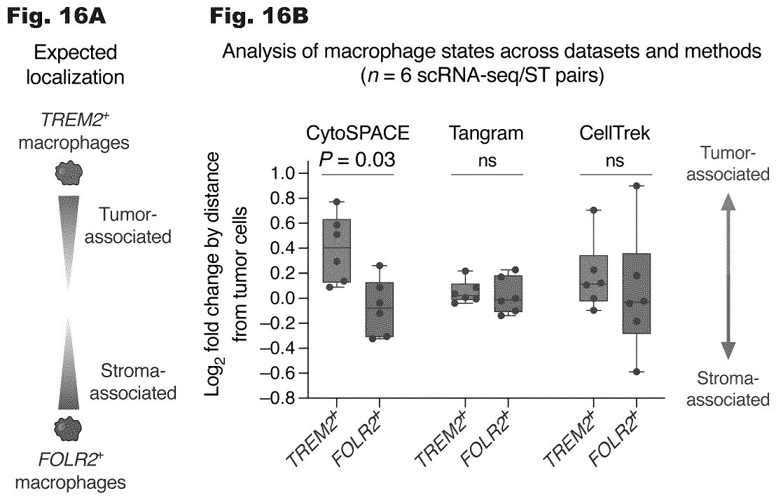

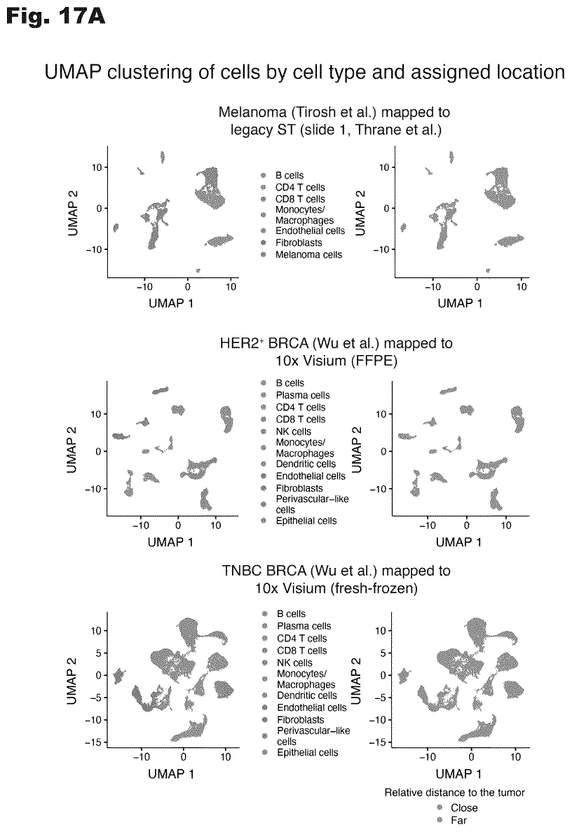

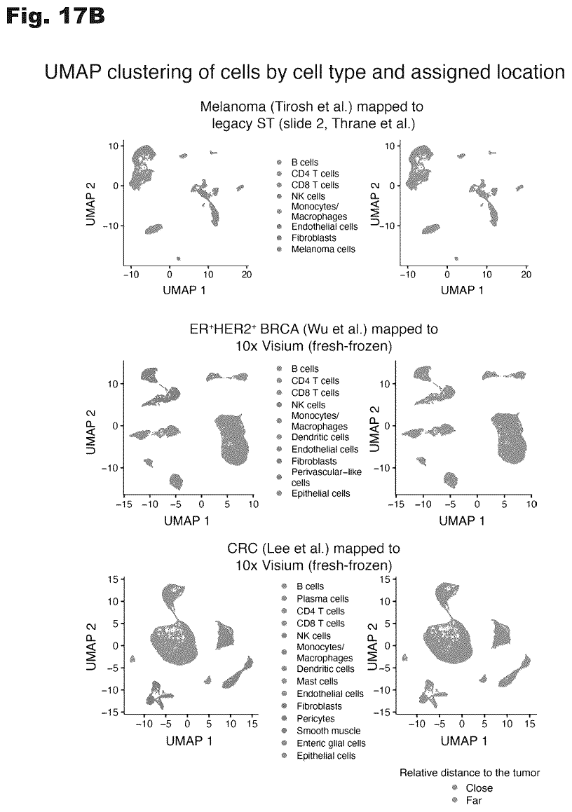

To demonstrate applicability to other spatially biased cell states, the analysis was extended to diverse TME lineages, identifying cell type-specific genes that vary in expression as a function of distance from tumor cells. To validate the results, two recently defined cellular ecosystem subtypes in human carcinoma, CE9 and CE10 we analyzed (for more on CE9 and CE10, see B. A. Luca, et al., Cell 184, 5482-5496.e5428 (2021), the disclosure of which is incorporated herein by reference). These “ecotypes,” which were also observed in melanoma, each encompass B cells, plasma cells, CD8 T cells, CD4 T cells, and monocytes/macrophages with stereotypical spatial localization. CE9 cell states are preferentially localized to the tumor core whereas CE10 states are preferentially localized to the tumor periphery. Using marker genes specific to each state, it was asked whether single cells mapped by each method were consistent with CE9 and CE10-specific patterns of spatial localization. Indeed, as observed for T cell exhaustion factors, CytoSPACE successfully recovered expected spatial biases in CE9 and CE10 cell states across lymphoid and myeloid lineages (FIG. 14B), outperforming 12 previous methods in both the magnitude and orientation of marker gene enrichments (FIGS. 14A, 14C and 14D). Furthermore, consistent with simulation experiments, CytoSPACE results remained robust to perturbations of its input parameters (FIGS. 15A to 15F). As further validation, predicted spatial localization patterns of TREM2+ and FOLR2⁺ macrophages were assessed, which were recently shown to localize to the tumor stroma and to the tumor mass, respectively, across diverse cancer types (FIG. 16A). Compared to Tangram and CellTrek, only CytoSPACE recapitulated these prior findings with statistical significance (FIG. 16B). Moreover, when inferred spatial locations (close to tumor vs. far from tumor) were projected onto UMAP embeddings of scRNA-seq data, single cells generally failed to cluster on the basis of their distance from tumor cells (FIGS. 17A and 17B). These data underscore the ability of CytoSPACE to accurately identify spatially resolved cell states, including those not discernible from scRNA-seq or ST data alone.

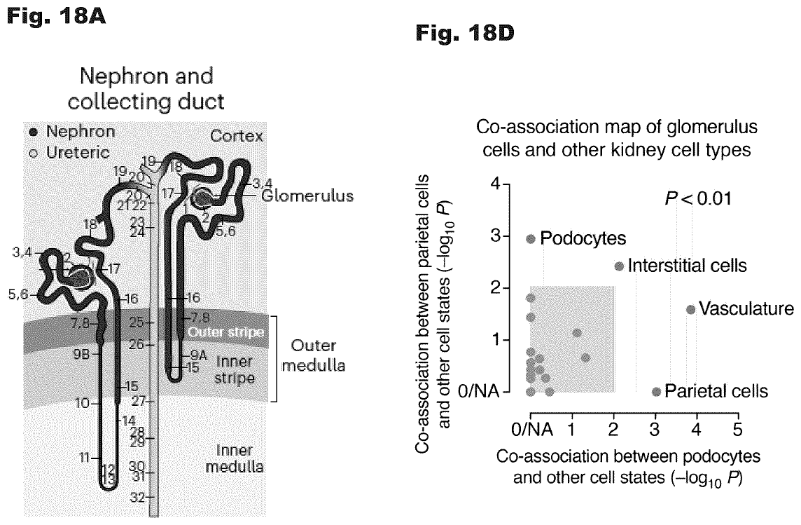

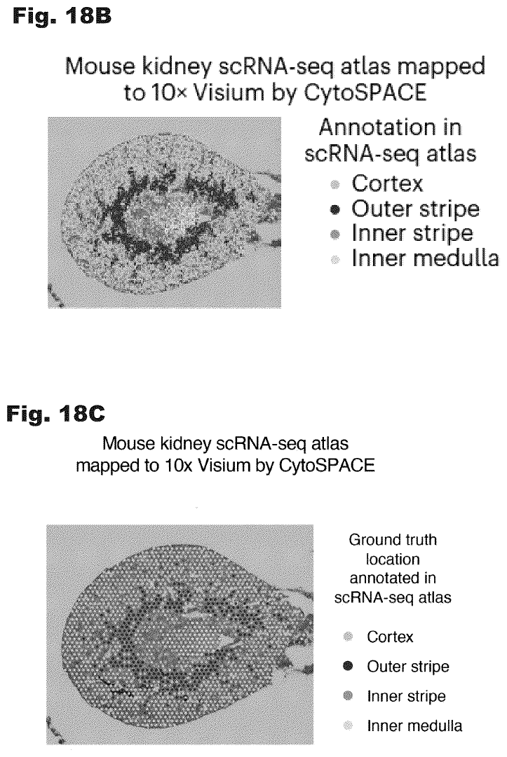

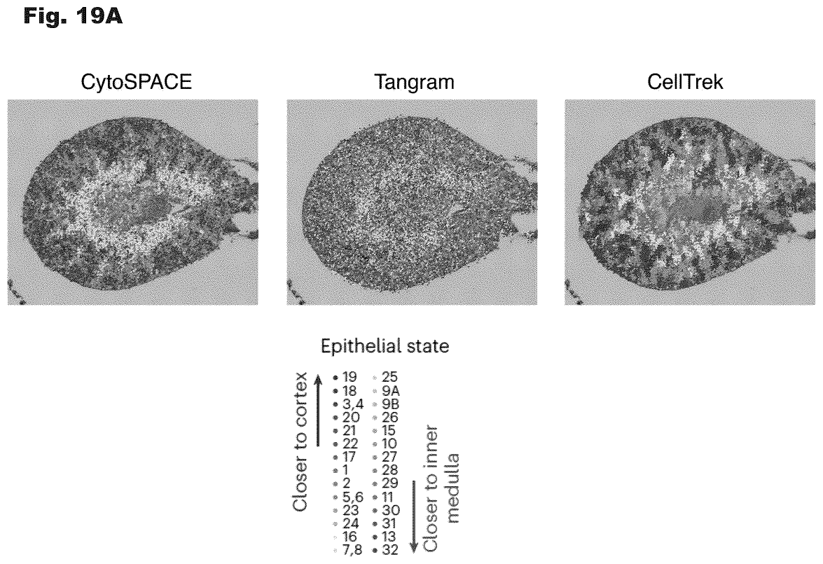

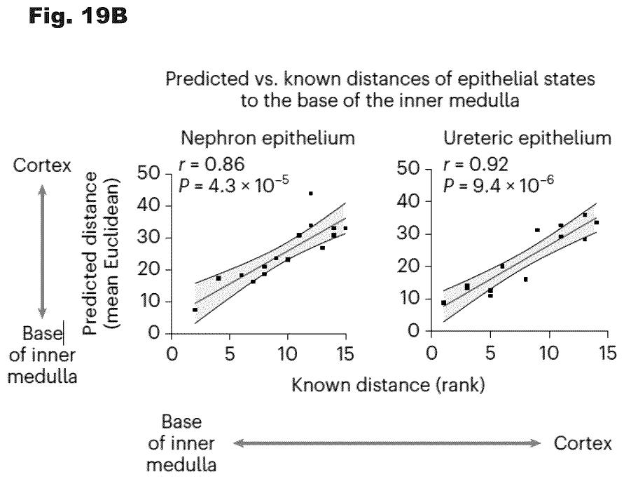

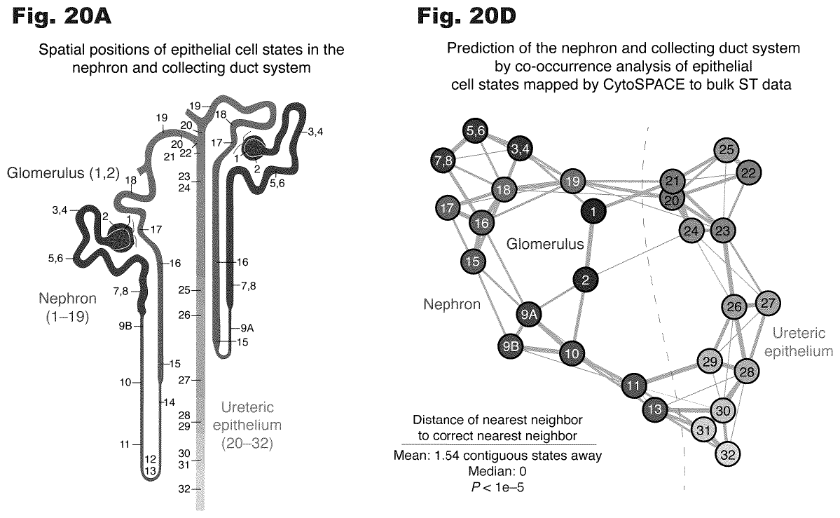

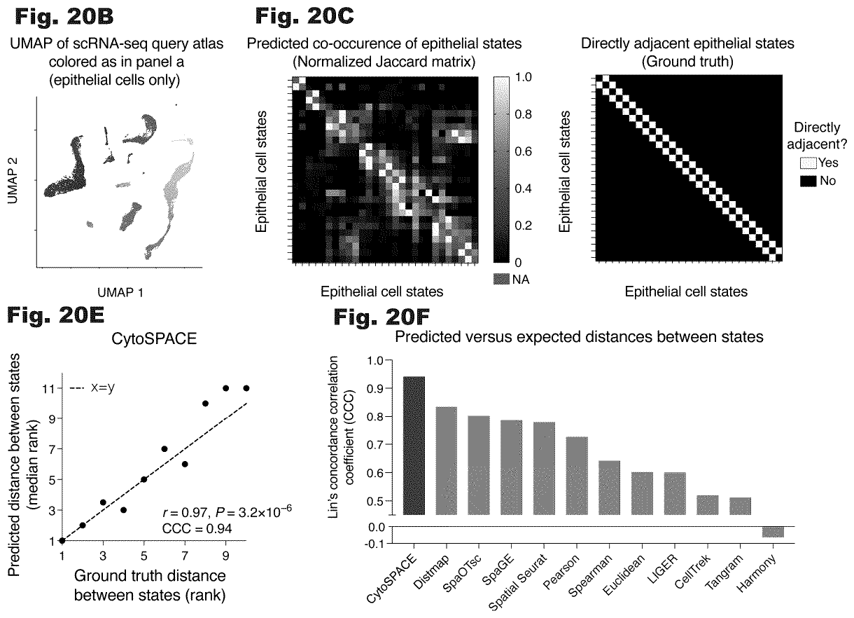

To further demonstrate how CytoSPACE can illuminate spatial biology, two additional scenarios were explored. First, it was asked whether CytoSPACE can uncover densely packed cellular substructures in bulk ST data. For this purpose, normal mouse kidney was selected, which has highly granular spatial architecture. After mapping a well-annotated scRNA-seq atlas with >30 spatially resolved subtypes of kidney epithelium to a 10× Visium profile of normal mouse kidney (55 μm diameter per spot) (FIG. 18A and Table 5), it was assessed whether CytoSPACE recapitulates known patterns of spatial organization. Indeed, CytoSPACE (i) reconstructed known zonal regions (FIGS. 18B and 18C), (ii) identified cell types that preferentially colocalize to the glomerulus (˜70 μm diameter; FIG. 18D), and (iii) arranged nearly 30 epithelial states in spots consistent with their known locations in the nephron epithelium and collecting duct system, outperforming previous methods (FIGS. 19A and 19B, and FIGS. 20A to 20F).

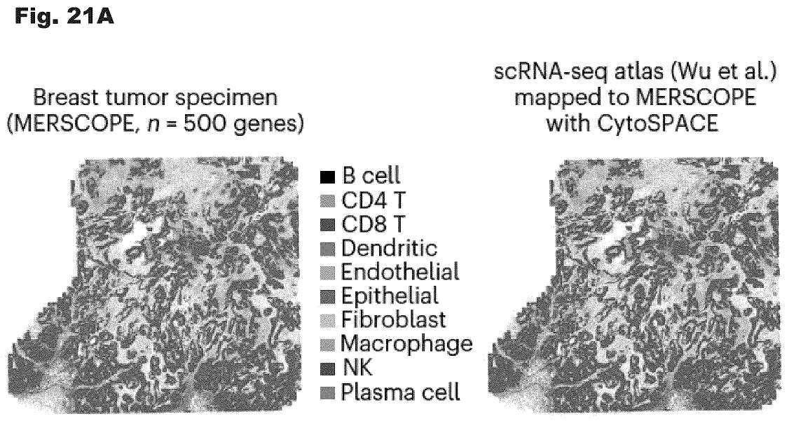

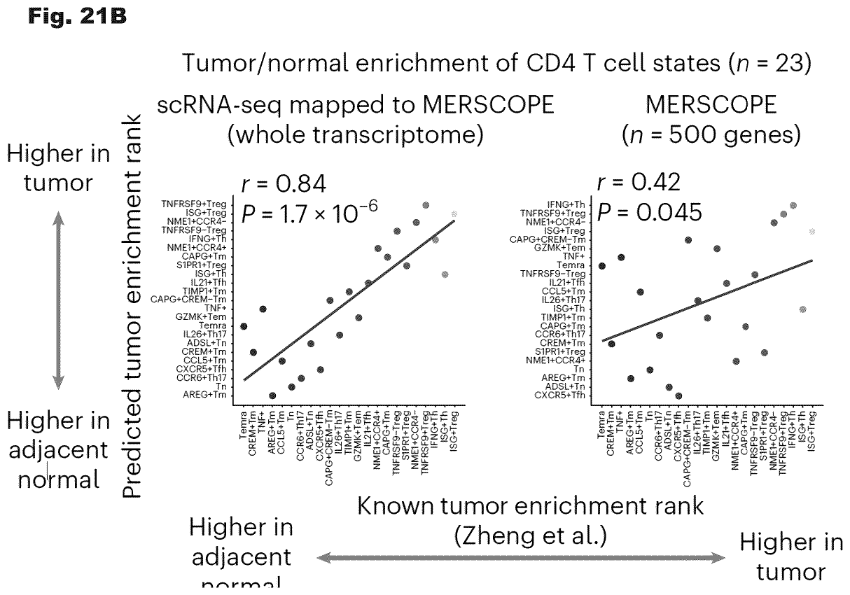

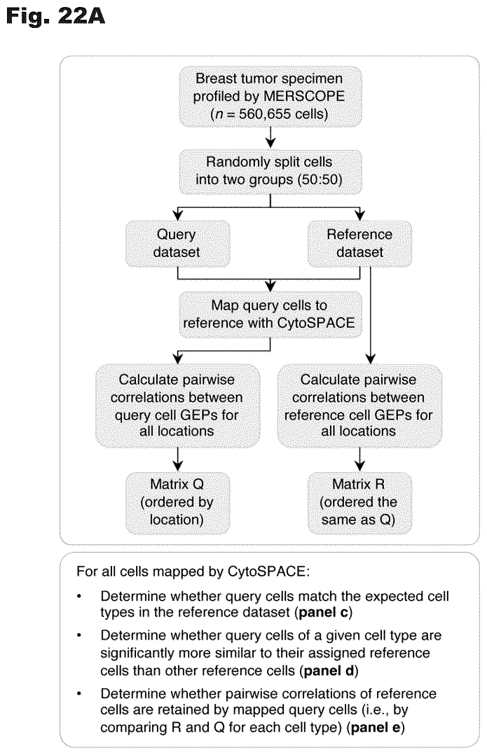

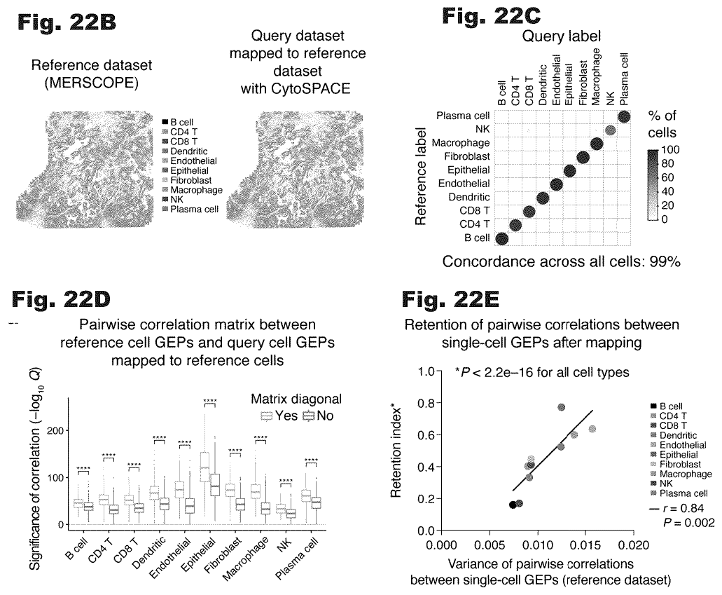

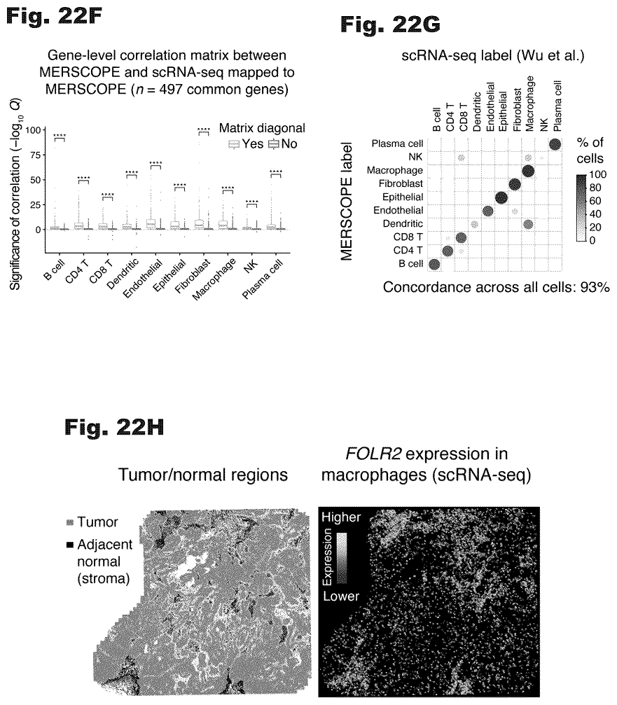

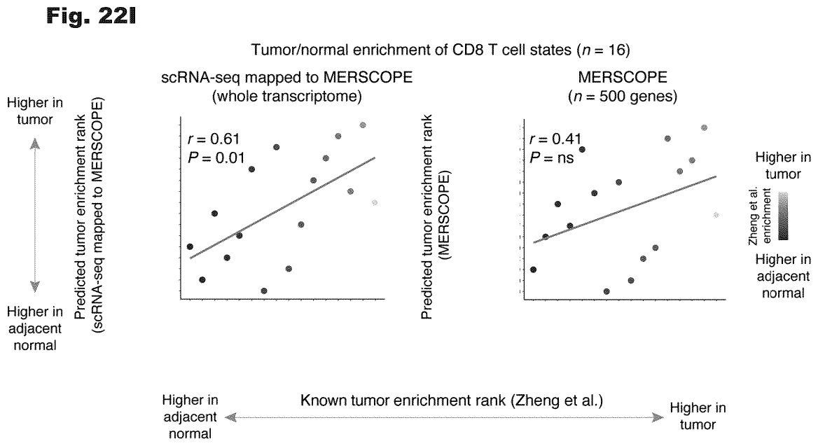

Finally, it was asked whether CytoSPACE can enhance single-cell ST datasets with low gene throughput. To do so, a breast cancer specimen was analyzed. The specimen contained >550 k annotatable cells and 500 preselected genes profiled by MERSCOPE (Vizgen). First, it was confirmed that CytoSPACE could accurately map single cells profiled by MERSCOPE and recapitulate their spatial dependencies (FIGS. 22A to 22E). Next, a scRNA-seq breast cancer atlas was mapped to the same MERSCOPE dataset. In addition to observing strong inter-platform agreement for most annotated cell types (FIG. 21A and FIGS. 22F and 22G), striking biases we observed in cancer-associated T cell signatures enriched in tumor or adjacent normal tissue (FIG. 21B, and FIGS. 22H and 221, and Table 6). Such enrichments were markedly more correlated with expected enrichments than those calculated from MERSCOPE data alone (FIG. 21B and FIG. 22I and Table 6). Collectively, these data emphasize the versatility of CytoSPACE for complex tissue reconstruction at the single-cell level.

CytoSPACE is a tool for aligning single-cell and spatial transcriptomes via global optimization. Unlike related methods, CytoSPACE ensures a globally optimal single-cell/spot alignment conditioned on a correlation-based cost function and the number of cells per spot. Moreover, it can be readily extended to accommodate additional constraints, such as the fractional composition of each cell type per spot (e.g., as inferred by RCTD or cell2location). In contrast, CellTrek is dependent on the co-embedding learned by Spatial Seurat, which can erase subtle, yet important biological signal (e.g., cell state differences). While Tangram is robust in idealized settings, it cannot guarantee a globally optimal solution. While CytoSPACE requires two input parameters, both parameters can be reasonably well-estimated using standard approaches, suggesting they are unlikely to pose a major barrier in practice. Furthermore, on both simulated and real datasets, CytoSPACE was substantially more accurate than related methods. As such, CytoSPACE is useful for deciphering single-cell spatial variation and community structure in diverse physiological and pathological settings.

### Examples of Methods

### CytoSPACE Analytical Framework

CytoSPACE leverages linear optimization to efficiently reconstruct ST data using single-cell transcriptomes from a reference scRNA-seq atlas. To formulate the assignment problem mapping individual cells in scRNA-seq data to spatial coordinates in ST data, let an N×C matrix A denote single-cell gene expression profiles with N genes and C cells; let an M×S matrix B denote gene expression profiles of spatial transcriptomics (ST) data with M genes and S spots; and let G be the vector of length g that contains the subset of desired genes shared by both data sets. For both gene expression profile matrices, values are first normalized to counts per million (or transcripts per million for platforms covering the full gene body) and then transferred into log₂ space. Thus, in its default implementation, CytoSPACE uses all genes as input and does not involve a dimension reduction step. Next, (by default) the number nₛ, s=1, . . . , S, of cells contributing RNA content in the s^{th} spot of ST data was estimated (see “Estimating the number of cells per spot”). It was assumed that the s^{th} spot contains nₛ sub-spots that can each be assigned to a single cell, and build an M×L matrix B by replicating the s^{th} column of B, nₛ times, where

$$L = {\sum }_{s = 1}^{S} {n}_{s}$$

denotes the total number of estimated sub-spots in the ST data. As described in the following sections, the scRNA-seq matrix A was sampled such that the total number of cells, with cell types represented according to their inferred fractional abundances, matches the total number of columns in B, yielding an N×K matrix Ā, where K=L. Next, define an assignment x:=[xₖₗ], 0≤xₖₗ≤1,k=1, . . . , K and l=1, . . . , L, where xₖₗ denotes the assignment of the k^{th} cell in the scRNA-seq data to the l^{th} sub-spot in the ST data. Of note, although xₖₗ is only explicitly constrained to real values within this range, a globally optimal solution will naturally satisfy xₖₗ ∈{0,1}. The optimal cell/sub-spot assignment x* that minimizes the following linear cost function was found by:

$${x}^{*} = arg min Cost ( x ) = arg min {\sum }_{k = 1}^{K} {\sum }_{l = 1}^{L} {d}_{kl} {x}_{kl} ,$$

subject to:

$${\sum }_{l = 1}^{L} {x}_{kl} = 1 , k = 1 , … , K , {\sum }_{k = 1}^{K} {x}_{kl} = 1 , l = 1 , … , L .$$

where dₖₗ denotes the distance between the gene expression profiles of the k^{th} cell and the l^{th} sub-spot. The above constraints guarantee that each cell is only assigned to one sub-spot and each sub-spot only receives one cell. In general, dₖₗ can be obtained using any metric that quantifies the similarity between the gene expression profiles of the reference and target data sets. Different similarity metrics were examined for simulated data and selected Pearson correlation as below due to its computational efficiency:

$${d}_{kl} = - corr ( {\overline{A}}_{k}^{G} , {\overline{B}}_{l}^{G} ) ,$$

where

$${\overline{A}}_{k}^{G} and {\overline{B}}_{l}^{G}$$

denote the k^{th} and l^{th} columns of expression matrices Ā and B̅, respectively, for the shared genes in G.

Two possible solvers were provided for CytoSPACE, both of which will return the globally optimal solution of the above problem as formulated. The first of these implements the shortest augmenting paths-based Jonker-Volgenant algorithm, in which the dual problem of the above formulation was defined as:

$$max ( {\sum }_{k = 1}^{K} {u}_{k} + {\sum }_{l = 1}^{L} {v}_{l} ) ,$$

subject to:

$${r}_{kl} := {d}_{kl} - ( {u}_{k} + {v}_{l} ) \geq 0 , l = 1 , … , L , k = 1 , … , K ,$$

where for the dual variables uₖ and vₗ, the reduced cost rₖₗ is defined as dₖₗ−(uₖ+vₗ). The dual problem reformulates the optimization task to find an alternative reduction of the cost function with maximum sum and non-negative reduced costs. In summary, this algorithm constructs the auxiliary network (or equivalently a bipartite graph) and determines from an unassigned row k to an unassigned column j an alternative path of minimal total reduced cost and uses it to augment the solution. In practice, despite time complexity O(L³), the Jonker-Volgenant algorithm is substantially faster than the majority of available algorithms for solving the assignment problem. By default, CytoSPACE calls the lapjv solver from the lapjv software package (version 1.3.14) in Python 3, which makes use of AVX2 intrinsics for speed (github.com/src-d/lapjv). With this solver, CytoSPACE runs in approximately 5 minutes on a single core using a 2.4 GHz Intel Core i9 chip for a standard 10× Visium sample with an estimated average of 5 cells per spot.

An alternate solver was based on the cost scaling push-relabel method using the Google OR-Tools software package in Python 3 (A. V. Goldberg and R. Kennedy, Math. Program. 71, 153-177 (1995), the disclosure of which is incorporated herein by reference). This solver is an integer programming approximation method in which exact costs are converted to integers with some loss of numerical precision and which runs with time complexity O(L² log(LC)), where C denotes the largest magnitude of an edge cost. In practice, this solver is approximately as fast as the Jonker-Volgenant based solver. However, for very large numbers of cells to be mapped, it can offer faster runtimes. Furthermore, it is supported more broadly across operating systems, so this solver may be useful for users working on systems which do not support AVX2 intrinsics as required by the lapjv solver. For users who wish to obtain the exact results of lapjv on operating systems that do not support the lapjv package, an equivalent but considerably slower solver implementing the Jonker-Volgenant algorithm is provided via the lap package (version 0.4.0), which has broad compatibility.

### Estimating Cell Type Fractions

To overcome variability in cell type fractional abundance between a given ST sample and a reference scRNA-seq dataset, the first step of CytoSPACE requires estimating cell type fractions in the ST sample (FIG. 3B). Of note, only global estimates for the entire ST array are required and these may be obtained by combining spot-level fractions by cell type. While an intriguing future extension of CytoSPACE would be to estimate cell type fractions as part of the optimization routine, many deconvolution methods have been proposed to determine cell type composition from ST spots, and any such method can be deployed for this purpose. In this example, Spatial Seurat from Seurat version 3.2.3 was used for the primary analyses and show that correlations between estimated and true fractions of distinct cell types are high in simulated data (FIG. 6A). After loading raw count matrices, SCTransform( ) and RunPCA( ) was performed with default parameters, followed by FindTransferAnchors( ) in which the preprocessed scRNA-seq and ST data served as the reference and query respectively. Spot-level predictions were obtained by TransferData( ) and global predictions were obtained by summing prediction scores per cell type across all spots and scaling the sum of cell type scores to one.

In addition to Spatial Seurat, the performance of RCTD was tested for estimating global cell type fractions as input to CytoSPACE (FIGS. 8A to 8C). RCTD version 2.0.0 (package spacexr in R) was employed with doublet_mode=‘full’ and otherwise default parameters to obtain cell type fraction estimates per spot, followed by summing spot normalized result weights per cell type across all spots and scaling the sum to one.

### Estimating the Number of Cells Per Spot

The number of detectably expressed genes per cell (‘gene counts’) tightly corresponds to total captured mRNA content, as measured by the sum of unique molecular identifiers (UMIs) per cell⁴⁵. As gene counts are routinely used as a proxy for doublets or multiplets in scRNA-seq experiments, it was hypothesized that the sum of UMIs per ST spot may reasonably approximate the number of cells per spot, as required for the second step of CytoSPACE (FIG. 3B). To test this hypothesis while blunting the effect of outliers, technical variation, and the impact of cell volume, UMIs were normalized to counts per million per spot and then performed log₂ adjustment. Then, the number of cells per ST spot was estimated by fitting a linear function through two points: for the first point, it was assumed that the minimum number of cells per spot is one and that this minimum in cell number corresponds to the minimum sum of UMIs in log₂ space. For the second point, it assumed that the mean number of cells per spot corresponds to the mean sum of UMIs in log₂ space and set this value according to user input. For 10× Visium samples in which spots generally contain 1-10+ cells per spot, a mean of 5 cells per spot was employed throughout this work. For legacy ST samples with larger spot dimensions, a mean of 20 cells per spot was selected. The number of cells for every spot was calculated from this fitted function. In support of this hypothesis, for simulated ST datasets, it was found that the Pearson correlation between the estimated and real number of cells ranged between 0.80 and 0.93, depending on the dataset and spot resolution evaluated, with log₂-adjustment outperforming the sum of UMIs in the original linear scale (i.e., without CPM) (FIGS. 6B to 6D). The same was true when comparing against the number of cells per spot analyzed by cell segmentation (VistoSeg) applied to previously analyzed imaging data from a mouse brain Visium sample (FIG. 6E), further validating the approach. While this estimation component is provided by default, users may also provide their own estimates for this step, including those generated by cell segmentation methods (e.g., VistoSeg, CellPose).

### Harmonizing the Number of Cells Per Cell Type

The third step of CytoSPACE equalizes the number of cells per cell type between the query scRNA-seq dataset and the target ST dataset (FIG. 3B). This is accomplished by sampling the former to match the predicted quantities in the latter using one of the following methods:
Duplication. Let num_{sc,k} and num_{ST,k} denote the real and estimated number of cells per cell type k in scRNA-seq and ST data, respectively. For cell type k, if num_{sc,k}<num_{ST,k}, CytoSPACE retains all available cells in the scRNA-seq data and, also, randomly samples num_{ST,k}−num_{sc,k} cells from the same num_{sc,k} cells. Otherwise, it randomly samples num_{ST,k} from the num_{sc,k} available cells with cell type label k in the scRNA-seq data. By default, CytoSPACE applies this method for real data to ensure all cells assigned are biologically appropriate.
Generation. Here, when num_{sc,k}<num_{ST,k}, instead of duplicating cells, new cells of a specific type are generated with independent random gene expression levels by sampling each gene from the gene expression distribution of cells of the same type uniformly at random. This method was used for benchmarking simulations to avoid bias in measuring precision owing to the presence of duplicated cells.

### Simulation Framework

To evaluate the accuracy and robustness of CytoSPACE (FIG. 4A), ST datasets with known single-cell composition were simulated using previously annotated Slide-seq datasets of mouse cerebellum and hippocampus sections. Let Sl be an M×B gene expression matrix of a Slide-seq puck with M genes and B beads. To create a higher gene coverage version of Sl, denoted Sc, previously annotated scRNA-seq datasets of the same brain regions were used to replace Sl beads with single-cell transcriptomes. Following quality control, in which outlier cells with >1,500 genes were removed, each bead in the Slide-seq datasets was matched with the nearest cell of the same cell type in the scRNA-seq dataset by Pearson correlation. This was done separately for each mouse brain region. As single cells may be matched with more than one bead, to obtain unique single-cell transcriptomes, genes were permuted between cells of the same cell type. For each cell, 20% of its transcriptome of genes randomly selected per cell was replaced with that of another randomly selected cell of the same cell type such that the latter is not a duplicate of the former. For simplicity, the number of beads present in the two tissues as matched by randomly sampling beads from the hippocampus data down to the number present in the cerebellum data.

Having created an Sc matrix for each brain region, it was next sought to generate ST datasets with defined spot resolution. For this purpose, an m×n spatial grid was imposed over the entire puck. In each grid spot xᵢⱼ, i=1, . . . , n, j=1, . . . , m, the sum was calculated of raw counts S̅c̅ᵢⱼ of the cells located within the grid-spot xᵢⱼ. Since the spatial resolution of ST data varies depending on the technology used, ST datasets were simulated with an average of 5, 15, and 30 cells per spot.

Finally, in order to (i) leverage the scRNA-seq data underlying each Sc matrix as a query dataset and (ii) emulate technical variation between platforms, noise was added to the scRNA-seq data in defined amounts. To this end, a percentage of genes p to perturb were selected, then a corresponding subset of genes from each cell was randomly selected to which noise was added from the exponentiated Gaussian distribution 2^{N(0,1)} Noise perturbations were considered for the following values of p: 5%, 10%, and 25%. Despite the addition of noise, UMAP plots of perturbed transcriptomes remained similar to the original data, implying maintenance of biologically realistic data structure (FIGS. 4C to 4F).

### Quality Control Considerations for Cell-to-Spot Alignment

There are two key scenarios in which mismatch between scRNA-seq and ST data can occur. In the first scenario, cell types are detectable in the scRNA-seq dataset but not in the spatial dataset. CytoSPACE addresses this issue by requiring cell type abundance estimates as input (e.g., using Seurat, RCTD, or cell2location. In doing so, cell types missing from the ST dataset will generally be omitted from the spatial mapping (if imputed with zero fractional abundance) or inferred with low fractional abundance, minimizing their impact on performance.

In the second scenario, cell types are detectable in the spatial dataset but not in the scRNA-seq dataset, leading to incorrect mapping. Except for cell types that are either rare or prone to dissociation-induced losses, this scenario is uncommon, as droplet sequencing can readily canvas all major cell types in a given tissue sample. Other methods for spatial spot decomposition, including Seurat, RCTD, and cell2location, have the same limitation, which is usually negligible in practice.

While the Jonker-Volgenant algorithm is guaranteed to optimally solve the assignment problem given its cost function, there is no underlying probabilistic framework for estimating mapping uncertainty. An alternative is to determine whether a given cell type belongs to a given spatial spot after mapping—that is, whether a spot contains at least one cell of the same cell type. Notably, this definition is considerably less demanding than the metric described in “Performance assessment”. Nevertheless, to explore this possibility, the following procedure was implemented: First, to identify the top marker genes for each cell type mapped by CytoSPACE, NormalizeData( ), ScaleData( ), and FindAllMarkers( ) from Seurat v4.0.1 were sequentially applied to the scRNA-seq query dataset using default parameters. Then, the ST dataset was normalized and scaled using the same workflow. For each cell type i with at least 5, and up to 50 marker genes (denoted by m) identified by −log₁₀ adjusted p-value with log₂ fold change >0, 50 spatial spots were randomly selected for which CytoSPACE assigned at least one cell of cell type i and 50 spatial spots without at least one cell of cell type i. If <50 spots satisfied a given condition, 50 spots were sampled with replacement. Next, cell-to-spot assignments were used to reconstitute each selected spot as a pseudo-bulk transcriptome from the normalized and scaled scRNA-seq dataset by averaging over the assigned cells. A support vector machine (e1071 v1.7.8 in R) was subsequently trained to distinguish the two groups of pseudo-bulks from the previous step using the top m marker genes of cell type i. With this model, the probability, termed a confidence score, that cell type i belongs to each spot in the normalized and scaled ST dataset was calculated. Finally, for each mapped cell of type i, its spot-specific confidence score was retrieved.

This approach was evaluated on simulated ST data where ground truth is known (FIG. 5A). Although the fraction of incorrectly mapped cells (defined as above) was already low prior to applying this filter (<5%), it successfully distinguished correctly- from incorrectly-mapped cells with high statistical significance, with nearly all AUCs exceeding 0.8 for classifying individual cell types (FIGS. 5B and 5C). Moreover, at a confidence threshold above 10%, virtually every correctly-mapped cell was retained whereas >75% of incorrectly-mapped cells were removed (FIGS. 5D and 5E). Thus, this procedure, which is available via the CytoSPACE GitHub repository, may be used as an optional post-processing step for exploring alignment quality.

Benchmarking Analysis with Simulated Datasets

To fully evaluate the performance of CytoSPACE, an extended benchmarking analysis including Tangram, CellTrek, and 10 additional methods that may be adapted was performed (FIG. 7C). Methods were included if the method (i) was applicable to a single-cell query dataset and spatial reference dataset, including bulk ST data; (ii) produced an output, or involve an intermediate step, in which the two datasets are aligned, allowing imputation of single-cell spatial coordinates in the query dataset (e.g., scRNA-seq integration techniques, some gene imputation methods, naïve distance metrics); and (iii) was peer-reviewed with a publicly available software implementation.

Most previous methods failed to satisfy these requirements, including methods designed for spot-level decomposition (e.g., cell2location, RCTD), spatial clustering (e.g., BayesSpace), and spatial coordinate prediction without a spatial reference (e.g., novoSpaRc). Accordingly, the benchmarking analysis consists of three dedicated cell-to-spot mapping methods (CytoSPACE, Tangram, CellTrek), three single-cell integration methods (Harmony, LIGER, and Seurat V3), four methods from which cell-to-spot assignments can be extracted (DistMap, SpaGE, DEEPsc, and SpaOTsc); and three naïve methods (Pearson correlation, Spearman correlation, and Euclidean distance). Below the application of each approach is described.

CytoSPACE. For each ST resolution and scRNA-seq noise level, the fractional abundance of known cell types in the ST sample was estimated via Spatial Seurat, as described in “Estimating cell type fractions”. CytoSPACE was run with the “generated cells” option and with the lapjv solver implemented in Python (package lapjv, version 1.3.14).

Tangram. Like CytoSPACE and in contrast to the other methods considered here, Tangram seeks to arrange input cells across spots optimally, and cell-to-spot mappings for each input cell are strongly inseparable from the cell-to-spot mappings of other cells. Thus, to ensure a fair comparison with CytoSPACE, Tangram (version 1.0.2) was run with the same input cells mapped by CytoSPACE, including cells newly generated after resampling to match predicted cell type numbers. It was also provided a normalized vector of CytoSPACE's cell number per spot estimate as the density prior (density_prior argument). Tangram was trained on CPM-normalized scRNA-seq data in two ways: (i) using all available genes per cell and (ii) using the top marker genes stratified by cell type. To identify marker genes using Seurat (version 4.1.0), NormalizeData( ) was applied with default parameters and FindAllMarkers( ) with only.pos=TRUE, min.pct=0.1, and logfc.threshold=0.25. The top 100 genes by average log₂ fold change were then selected for each cell type.

CellTrek. Given that CellTrek heavily duplicates input cells (by default) and also filters input cells based on whether mutual-nearest neighbors are identified between cells and spots, CellTrek (version 0.0.0.9000) was provided with all cells present in each simulated ST dataset (without the newly generated cells mapped by CytoSPACE and Tangram). After single cells were assigned to spatial coordinates, the closest ST spot for each cell was selected via Euclidean distance. As the CellTrek wrapper does not handle ST input without associated h5 and image files, the code was modified to accommodate ST datasets from other sources. CellTrek was run with default parameters, with the exception of (i) limiting the repel functionality (repel_r=0.0001), as this parameter forces imputed spatial coordinates to arbitrarily deviate from their original predictions, and (ii) setting spot_n to twice the mean number of cells per spot for each spatial resolution tested.

DistMap. DistMap seeks to computationally reconstruct ST data at single-cell resolution from paired scRNA-seq. It uses marker genes and a binarization approach calculating Matthews correlation coefficients to obtain distributed positional assignments for each cell”.

For benchmarking, DistMap (v0.1.1) was provided with all input cells and spots, restricting genes to marker genes (selected as described for benchmarking Tangram with top genes) expressed in at least 5 cells and 5 spots. Count matrices were CPM-normalized and log₂-adjusted. Following creation of a DistMap object with the normalized ST data provided for the insitu argument, the scRNA-seq data were binarized via binarizeSingleCellData(dm, seq(0.15, 0.5, 0.01)). A binarized version of the ST data matrix was prepared by setting all nonzero counts to one, then the insitu.matrix member variable of the DistMap object was replaced with this binarized version. The cell-to-spot mapping was performed with mapCells( ) and each cell was assigned to the spot with highest score as returned in the mcc.scores member variable.

SpaOTsc. SpaOTsc is a method for inferring spatial properties of scRNA-seq data, designed primarily for the investigation of spatial cell-cell communications. As the first step in this process, SpaOTsc computes a map between single cells and a spatial dataset using an optimal transport approach on marker genes.

For benchmarking, SpaOTsc (v0.2) was provided with all input cells and spots, restricting genes to marker genes (selected as described for benchmarking Tangram with top genes) expressed in at least 5 cells and 5 spots. Following tutorial instructions, SpaOTsc was implemented as follows. First, counts were normalized to sum to 10,000 per cell or spot respectively and then the resulting scRNA-seq (df_sc) and ST (df_is) matrices were log₂-transformed. From the normalized scRNA-seq data, principal component analysis (PCA) was performed with prcomp in R, then the Pearson correlation coefficient matrix (sc_pcc) was computed between single cells from the top 40 principal components. To obtain a Matthews correlation coefficient matrix (mcc) between cells and spots, each normalized data matrix was binarized (resulting in df_sc_bin and df_is_bin for scRNA-seq and ST matrices, respectively) with a quantile threshold of 0.7, then computed the Pearson correlation coefficient over all cell-spot pairs. Then SpaOTsc was run with the following set of commands: C=np.exp(1−mcc), issc=SpaOTsc.spatial_sc(sc_data=df_sc, sc_data_bin=df_sc_bin, is_data=df_is, is_data_bin=df_is_bin, sc_dmat=np.exp(1−sc_pcc), is_dmat=is_dmat), out=issc.transport_plan(C**2, alpha=0.1, rho=100.0, epsilon=1.0, cor_matrix=mcc, scaling=False). Each cell was then assigned to the spot with the highest score as returned in the output of issc.transport_plan( ).

DEEPsc. DEEPsc is a deep-learning based method for imputing spatial information onto scRNA-seq data given a spatial reference atlas. DEEPsc first transfers the spatial reference atlas data to a space of reduced dimensionality via PCA, then performs network training over it. The scRNA-seq data is projected into the same PCA space and fed into the DEEPsc network, which outputs a matrix of likelihoods that each cell originated from each spot in the ST tissue.

For benchmarking, DEEPsc (version number not available; last GitHub commit when cloned: Jun. 5, 2022) was provided with all input cells and spots, with each input matrix CPM-normalized then log-transformed via log 1p, and with genes restricted to those present in both matrices. DEEPsc was run with 50,000 iterations in parallel mode for training and with otherwise default parameters.

SpaGE. SpaGE, or Spatial Gene Enhancement using scRNA-seq, is a method for increasing gene coverage in ST measurements by integrating spatial data with higher coverage scRNA-seq datasets. SpaGE uses the domain adaptation algorithm PRECISE to project datasets into a shared space, in which gene expression predictions are then computed through a k-nearest neighbors approach. Although SpaGE was designed for gene expression prediction rather than mapping cells to spots, as it includes an integration step, it is possible to use this integration space for cell-to-spot mapping.

To do so while making full use of the SpaGE framework (version number not available; last GitHub commit when cloned: Jul. 20, 2021), a command to return the single nearest spot neighbor for each cell in the SpaGE integrated space was added to the source code. Then the modified SpaGE code was provided with all input cells and spots. Following the tutorial recommendation, genes not expressed in at least 10 cells were excluded, then CPM-normalized and log₂-transformed the scRNA-seq matrix, while normalizing the ST matrix to median counts per spot followed by log₂-transformation. SpaGE was run with n_pv=30, again per tutorial recommendation, and otherwise default parameters.

Spatial Seurat. Seurat, a well-known method for integrating single-cell expression datasets that works by identifying “anchors” between datasets, can be used with spatial data as well. Spatial Seurat integration for assigning cells to spots using Seurat v3 was tested. After loading scRNA-seq and ST count matrices into Seurat objects, the scRNA-seq and ST count matrices were preprocessed with SCTranform( ) and then assessed with the standard integration protocol of FindTransferAnchors(normalization.method=“SCT”) followed by TransferData( ). Cell-to-spot assignments were determined by the predicted.id returned from the resulting predictions assay.

Harmony. Harmony is a method for integrating multiple scRNA-seq datasets into a joint embedding space, employing clustering methods over principal component representations of the data to obtain linear correction factors for integration. As a dataset integration method, Harmony does not provide direct cell-to-spot mapping results. Thus, for benchmarking, the method was used to first integrate the full single cell and corresponding spatial datasets, then assigned each cell to its nearest spot within the integration space by selecting the spot with minimum Euclidean distance to the cell.

To obtain the integration space representations, the standard Harmony protocol was followed. First Seurat objects created from the scRNA-seq and ST count matrices were merged, then the standard Seurat processing pipeline of NormalizeData( ), FindVariableFeatures( ), ScaleData( ), and RunPCA( ), were each applied with default parameters. With the resulting Seurat object, Harmony v0.1 was run with group.by.vars=“orig.ident” and otherwise default parameters.

LIGER. Like Harmony, LIGER is another method designed for single-cell expression dataset integration, though LIGER relies instead on an integrative non-negative matrix factorization approach to embed features in a low-dimensional space, incorporating both dataset-specific and shared factors. As described above for Harmony, LIGER was used to obtain a shared embedding space between the scRNA-seq and ST datasets and then cells were assigned to spots according to minimum Euclidean distance.

To run LIGER (v.0.0), a LIGER object was created and processed with package functions normalize( ), selectGenes(var.thresh=0.2), and scaleNotCenter( ), for normalization, gene selection, and scaling respectively, and then applied using online_iNMF( ) and quantile_norm( ) to align the datasets. All parameters not specified here were set to defaults. Embeddings were extracted from the LIGER object member variable H.norm.

In addition to the above methods, Euclidean distance (calculated with the spatial.distance.cdist function of scipy v1.8.0), Pearson correlation, and Spearman correlation were assessed. Here, each cell was assigned to the spot that either minimized distance (Euclidean distance) or maximized correlation (Pearson and Spearman correlations). All ground truth cells were evaluated without resampling and input datasets were CPM normalized and log₂-adjusted prior to analysis.

Performance assessment. To determine the accuracy of single-cell mapping, assigned locations that exactly matched ground truth spots were classified as correct. Letting TP_{sc} denote the number of correct assignments, single-cell precision (Pr_{sc}) was defined as

$${Pr}_{sc} = \frac{{TP}_{sc}}{No . unique mapped cells with ground truth locations}$$

Of note, since generated cells (see “Harmonizing the number of cells per cell type”) did not have a corresponding ground truth location, they were excluded from this calculation. Separately, although CellTrek can assign the same cell ID i to multiple spots, any cell of ID i mapped to the correct spot at least once was considered correct. This was done without inflating the denominator or penalizing incorrect mappings for other cells with ID i.

### Measuring Robustness of CytoSPACE in Simulation

To be broadly useful, a computational method such as CytoSPACE must exhibit robustness to reasonable variation or error in inputs. With this in mind, CytoSPACE's consistency and robustness to variation was tested across input parameters.

Robustness to cell fraction estimation error. To mimic realistic technical error in estimating cell type fractions, in which proportionally larger error can be expected for rarer cell types, multiplicative noise was introduced within a four-fold range, with noise inversely dependent upon the original fraction estimate. First, for each cell type i in a sample, yᵢ was randomly sampled from a Gaussian distribution with mean zero and standard deviation inversely dependent on the original fraction estimate xᵢ for cell type:

$$σ = \frac{1}{2 {x}_{i}^{1 / 3}} , {y}_{i} ∼ N ( 0 , {σ}^{2} )$$

Here, the cubic root smooths the distribution toward the four-fold perturbation range desired. To restrict the range strictly to within a four-fold perturbation, a maximum absolute value of two was imposed on the resulting value:

$${z}_{i} = max ( - 2 , min ( 2 , {y}_{i} ) )$$

The perturbation of each original estimate was then computed as

$${\overline{x}}_{ι} = {x}_{i} \cdot {2}^{{z}_{i}}$$

with the resulting values then renormalized to unit sum.

CytoSPACE was tested with this noise model in simulation with five replicates for each simulated test case (“Simulation framework”), evaluating results via single-cell assignment precision as described in “Performance assessment” (FIGS. 10A and 10B).

Robustness to cell number per spot estimation error. Noise was introduced to estimates of number of cells per spot with a similar protocol to that described above for perturbing cell type fraction estimates. First, for each spot in a sample, yᵢ was randomly sampled from a Gaussian distribution with mean zero and standard deviation inversely dependent on the original estimate nᵢ for cell type i:

$$σ = \frac{p}{{n}_{i}^{1 / 3}} , {y}_{i} ∼ N ( 0 , {σ}^{2} )$$

In the above distribution, p denotes a tuning parameter which was set by spatial resolution in such a way as to produce similar Pearson correlations between the original and perturbed estimate as observed between the CytoSPACE estimate, based on RNA content, and the VistoSeg estimate, based on image segmentation (within the range of 0.50 to 0.55; FIG. 6E). To achieve this, p was set to 1.4 (simulated data with estimated mean of 5 cells per spot), 1.7 (simulated mouse cerebellum data with estimated mean of 15 cells per spot), 2.2 (simulated mouse cerebellum data with estimated mean of 30 cells per spot), 2.6 (simulated mouse hippocampus data with estimated mean of 15 cells per spot), and 3.7 (simulated mouse hippocampus data with estimated mean of 30 cells per spot).

To restrict the range of values to a feasible region, a minimum number of cells per spot of one and a maximum number of cells per spot of 110% of the original maximum M was imposed. The perturbed values n̅₁̅ were thus computed as

$$\overline{{n}_{ι}} = max ( 1 , min ( {n}_{i} \cdot Round ( {2}^{{y}_{i}} ) , 1.1 M ) )$$

CytoSPACE was tested with this noise model in simulation with five replicates for each simulated test case (“Simulation framework”), evaluating results via single-cell assignment precision as described in “Performance assessment” (FIGS. 10C to 10E).

Robustness to sampling variation. While most steps of the algorithm are deterministic, CytoSPACE requires that the input scRNA-seq dataset be resampled to create a pool of cells matching those expected in the ST dataset; this sampling is done at random. To test consistency of results across different samples, CytoSPACE was run ten times with different seeds for each simulation case described in “Simulation framework.” Single-cell precision of the assignment was calculated as described above (“Performance assessment”). Results for this analysis are shown in FIG. 11A.

Robustness to distance metric. In addition to Pearson correlation, the default distance metric for CytoSPACE was implemented, CytoSPACE performance was tested with alternative distance metrics Spearman correlation and Euclidean distance as shown in FIG. 11B. For each ST resolution and scRNA-seq noise level in simulated data (as described in “Simulation framework”), CytoSPACE was run with Spearman correlation and Euclidean distance substituted for the distance metric.

### ST Datasets for TME Community Analysis

Melanoma ST data generated by Thrane et al.³⁵ were downloaded from spatialresearch.org/resources-published-datasets/doi-10-1158-0008-5472-can-18-0747/(K. Thrane, et al., Cancer Research 78, 5970-5979 (2018), the disclosure of which is incorporated herein by reference). Pre-processed spatial transcriptomics datasets of breast cancer (Visium fresh-frozen and FFPE) and colorectal cancer (fresh-frozen) specimens were downloaded from 10× Genomics (www.10×genomics.com/spatial-transcriptomics/). Annotations of regions containing tumor cells were downloaded from 10× Genomics for the Visium FFPE breast cancer sample and shared by 10× Genomics upon request for the Visium fresh-frozen breast cancer sample analyzed in this work. A pre-processed Visium array of a fresh/frozen TNBC specimen (1160920F) was obtained from Wu et al.³⁷ along with tumor boundaries (S. Z. Wu, et al, Nature Genetics 53, 1334-1347 (2021), the disclosure of which is incorporated herein by reference).

scRNA-Seq Tumor Atlases

All analyzed tumor scRNA-seq data, which were downloaded as preprocessed count (UMI-based) or transcript (non-UMI-based) matrices, were selected and curated to clinically-match the ST specimens analyzed in this work (see “Molecular classification of breast cancer specimens”). Additionally, author-supplied annotations were used for all scRNA-seq reference datasets, with the following modifications. For the melanoma dataset generated by Tirosh et al., we excluded normal melanocytes and divided T cells into CD4 and CD8 subsets by the expression of CD8A/CD8B and CD4/IL7R (I. Tirosh, et al., Science 352, 189-196 (2016), the disclosure of which is incorporated herein by reference). For the breast cancer dataset from Wu et al. and in the colorectal cancer dataset from Lee et al. (H. O. Lee, et al., Nature Genetics 52, 594-603 (2020), the disclosure of which is incorporated herein by reference), the authors' annotations were mapped to cell types according to the scheme in Table 2. Of note, T cells that could not be confidently classified as CD8 or CD4 T cells and myeloid cells that could not be confidently classified as monocytes/macrophages or dendritic cells were excluded.

### Molecular Classification of Breast Cancer Specimens

When available, author annotations were used to determine estrogen receptor (ER) and human epidermal growth factor receptor 2 (HER2) enrichment status for each scRNA-seq and ST tissue breast cancer sample. For the FFPE breast cancer specimen from 10× Genomics without receptor status annotation, the expression of ESR1 (ER) and ERBB2 (HER2) genes was examined. The FFPE breast cancer ST specimen as HER2+/ER− was reclassified based on high expression of ERBB2 without appreciable ESR1 expression.

Mapping of Single-Cell Transcriptomes onto Tumor ST Samples

For the various analyses herein, CytoSPACE and the other benchmarking methods described in “Benchmarking analysis with simulated datasets” were applied as follows:
CytoSPACE. Cell type fractions were computed using Spatial Seurat (“Estimating cell type fractions”) and CytoSPACE was run with the “duplicated cells” option and the lapjv solver as implemented in the lapjv Python package on a single CPU core. For all Visium samples, the mean number of cells per spot was set to 5, while for legacy ST samples (melanoma ST data), this parameter was set to 20.
Tangram. As input, the same single-cell transcriptomes mapped by CytoSPACE were analyzed, including duplicates, along with a density prior (density_prior argument) as determined by the number of cells per spot estimated by CytoSPACE. Since Tangram performed best with all genes when used for simulated ST datasets, Tangram (version 1.0.2) was run on CPM-normalized scRNA-seq data with 24 CPU cores on all available genes. Other parameters were set to default.
Cel/Trek. Given CellTrek's internal filtering mechanism (see “Benchmarking analysis with simulated datasets”), all cells in the corresponding scRNA-seq atlases were provided as input (without duplication or down-sampling). For Visium samples, CellTrek (version 0.0.0.9000) was run with default parameters with 24 CPU cores (reduction=‘pca’, intp=T, intp_pnt=10000, intp_lin=F, nPCs=30, ntree=1000, dist_thresh=0.4, top_spot=10, spot_n=10, repel_r=5, repel_iter=10, keep_model=T) and then assigned cells from raw output coordinates to their nearest spot by Euclidean distance. For the legacy ST samples (melanoma), the code was modified to handle inputs without h5 and image files, as detailed above. To fit the larger spot resolution in the legacy ST datasets, spot_n was fixed to 40. Other parameters were the same as above.
Other methods. The other benchmarking methods (DistMap, SpaOTsc, DEEPsc, SpaGE, Spatial Seurat, Harmony, LIGER, Euclidean distance, Pearson correlation, and Spearman correlation were implemented according to the details described in their corresponding sections in “Benchmarking analysis with simulated datasets,” with the following exception: for computational feasibility over especially large scRNA-seq datasets, SpaOTsc was run for two scRNA-seq/ST pairs (CRC and TNBC) with the protocol described above for “Tangram,” providing the cells mapped by CytoSPACE rather than the entire scRNA-seq dataset.

### Running Time Analysis

To evaluate the efficiency of CytoSPACE in practice and benchmark against recent dedicated cell-to-spot mapping methods, running times were recorded for CytoSPACE, Tangram, and CellTrek across all scRNA-seq tumor atlas/ST pairs tested (n=4 pairs with Visium ST data, n=2 pairs with lower resolution legacy ST data) (Table 3) with parameter details as described above. For CytoSPACE, running times was reported for both exact (shortest augmenting path via the lapjv solver) and integer approximation solvers, and both with and without a Spatial Seurat preprocessing step for obtaining input cell type fractional abundances. Data loading and file writing steps were excluded from running times for all methods. Methods were tested on comparable though not identical systems, with CytoSPACE, Spatial Seurat preprocessing steps, and Tangram tested on a computing cluster providing Intel E5-2640v4 (2.4 GHz base and 3.4 GHz max frequencies, with an associated 128 GB RAM), Intel 5118 (2.3 GHz base and 3.2 GHz max frequencies, with an associated 191 GB RAM), and AMD 7502 (2.5 GHz base and 3.35 GHz max frequencies, with an associated 256 GB RAM) processors, and with CellTrek tested on a server with an Intel E5-2680v3 processor and an associated 230 GB RAM. With the exception of CytoSPACE, in which the core mapping function uses only a single core, all methods were provided with 24 cores.

### Validation of Alternative Solver

To verify that the integer approximation solver is a fast alternative to the recommended exact solver (lapjv) yields comparable results, the proportion of single cells mapped to the same location was measured across the two solver methods. For each scRNA-seq tumor atlas/ST pair tested, the same single cells were mapped after preprocessing for duplication and downsampling to match the estimated cell type fractions in tissue via CytoSPACE with exact and integer approximation solvers, and the percentage of cells mapped to the same spot in each method were report (Table 4). For duplicated cells, no distinction was made between the copies.

### Spatial Enrichment Analysis

To determine whether single cells mapped to ST spots showed enrichment of known spatially-resolved gene expression programs, cells were first partitioned into two groups (‘close’ and ‘far’) based on their distance from cancer cells. For breast cancer ST samples, all of which were profiled by 10× Visium, tumor boundary annotations determined by a pathologist were used in order to group cells. For melanoma and CRC datasets, the mean Euclidean distance of each TME cell to the nearest five tumor cells (mapped by the respective alignment method) was determined. For the melanoma dataset, melanoma cells were considered as tumor cells, while in the CRC dataset, tumor epithelial cells were considered for the purpose of identifying tumor locations in tissue. For each TME cell type, the resulting distances were median-stratified into ‘close’ and ‘far’ groups. This was done for two main reasons. First, the CRC sample lacked tumor boundary annotations. Second, while melanoma datasets included such annotations, the low spatial resolution of the legacy ST platform prevented precise co-registration with spatial spots at the tumor/stroma interface.

To quantify spatial enrichment, pre-ranked gene set enrichment analysis (GSEA) was implemented in fgsea (v1.14.0) with nperm=10000. As input, all spatially-mapped single-cell transcriptomes were loaded by cell type into Seurat v4.1.0 (min·cells=5) and normalized with NormalizeData( ). For each method and cell type, a gene list ranked by log₂ fold-change was generated for the identity classes “near” and “far” using FoldChange( ). If fewer than 10 cells of a cell type were assigned to spots within one partition by at least one method, that cell type was excluded from the enrichment analysis. Of note, several methods (SpaOTsc, DEEPsc, Seurat, Hamony, and Euclidean distance) failed to map all evaluated cell types to regions both closer and farther from tumor cells, precluding the use of GSEA (as described below in “Spatial enrichment analysis”) on the affected cell types. In such cases, statistical comparisons to CytoSPACE were performed ignoring NAs. As CytoSPACE and Tangram were each run with the same scRNA-seq input, prior to running Seurat and fgsea, random sampling of cells were mapped by all other methods in order to match the number of cells per cell type mapped by CytoSPACE and Tangram and ensure a fair comparison among methods. This was done as described in “Harmonizing the number of cells per cell type—Duplication”. Gene sets for T cell exhaustion and CE9/CE10-associated cell states were derived by Zheng et al. and Luca et al., respectively (C. Zheng, et al., Cell 169, 1342-1356.e1316 (2017); and B. A. Luca Cell 184, 5577-5592.e5518 (2021); the disclosures of which are each incorporated herein by reference).

### Measuring Robustness of CytoSPACE on Real Data

The robustness testing described previously in “Measuring robustness of CytoSPACE on simulated data” was repeated with real data, applying CytoSPACE under various perturbations to the task of spatial enrichment analysis in TME samples and quantifying performance according to the recovery of expected spatial enrichments of gene sets in the TME as described in “Spatial enrichment analysis” (FIGS. 15A to 15F). The perturbation analyses were conducted in the same manner as with simulated data, except for the robustness to cell number per spot estimation error analysis, for which the tuning parameter p was set for scRNA-seq/ST dataset pairs as follows: 1.4 (Visium data), 1.9 (legacy ST data, melanoma slide 2), and 2.3 (legacy ST data, melanoma slide 1).

### Spatially Resolved Macrophage States

To evaluate the spatial localization of TREM2⁺ and FOLR2⁺ macrophages (FIGS. 16A and 16B), single-cell transcriptomes annotated as “Macrophages/Monocytes” were mapped to ST spots as described above (“Mapping of single-cell transcriptomes onto tumor ST samples”,) and ordered based on their spatial distance (Euclidean) from tumor cells. All cells were processed with Seurat as described in “Spatial enrichment analysis”. To calculate distance, the same metric described for melanoma and CRC datasets was used (“Spatial enrichment analysis”). For cells mapped within tumor boundaries annotated by a pathologist (breast cancer datasets), distances were set to zero. Then, cells were divided into ‘near’ (distance=0) and ‘far’ (distance >0) groups and the log₂ fold change of each gene was calculated using FoldChange( ) in Seurat (FIG. 16B).

### Integrative Single-Cell Spatial Analysis of Healthy Mouse Kidney

For the analyses on the healthy mouse kidney, the following downloaded: (i) a well-annotated scRNA-seq atlas encompassing immune cells, stromal elements, and >30 spatially resolved subtypes of kidney epithelium and (ii) a 10× Visium sample of normal mouse kidney. Kidney epithelial cell states lacking a numeric identifier (as in FIG. 18A) were omitted and states corresponding to the same phenotype were merged (3 and 4, 5 and 6, 7 and 8). The datasets were subsequently aligned with CytoSPACE as described in “Mapping of single-cell transcriptomes onto tumor ST samples” but with the mean number of cells per spot set to 10. Using epithelial cells, which have ground truth locations in the scRNA-seq atlas, the following zonal regions were analyzed: cortex (outermost region), outer medulla (central region), and inner medulla (innermost region), with the outer medulla further subdivided into the outer stripe (proximal to the cortex) and inner stripe (proximal to the inner medulla) (FIGS. 18B and 18C).

A ground truth rank was established for each epithelial cell state, reflecting its relative distance to epithelial state 32 (“deep medullary epithelium of pelvis”), which corresponds to the base of the ureteric epithelium (UE) in the inner medulla as previously reported (FIG. 18A and Table 5). Then, using single-cell spatial coordinates determined by CytoSPACE, the mean Euclidean distance of each epithelial cell state to the centroid of epithelial cells mapped to epithelial state was calculated. Regardless of whether nephron or UE was examined, correlations between predicted and ground truth distances were high, demonstrating CytoSPACE's potential for granular mapping (FIGS. 19A to 19D).

For the analysis in FIGS. 20A to 20E, it was tested whether CytoSPACE can resolve the known structure of the nephron and UE collecting system (FIG. 20A), which is not discernible from the scRNA-seq atlas (FIG. 20B) or ST dataset alone. For this purpose, spatial spots were scored as 1 if at least one cell of a given cell type was mapped by CytoSPACE and 0 otherwise. Then the resulting binary square matrix, with cell types as rows and cell types as columns, was converted into a Jaccard similarity matrix J that quantifies spatial overlap among epithelial states (FIG. 20C, left). After filtering all but the four nearest neighbors of each epithelial state in J, each row was converted to rank space and created an undirected graph from the data using igraph v1.2.6 in R. Then the graph was visualized using layout_with_fr( ), the Fruchterman and ReingoId force-directed layout algorithm implemented in igraph (FIG. 20D). To determine statistical significance (FIG. 20D), a permutation approach was devised in which the nearest neighbor Nᵢ of each epithelial state i in J was first determined. Then the minimum number of physically adjacent epithelial states (denoted by xᵢ) between Nᵢ and the ground truth nearest neighbor(s) of i was calculated (FIG. 20C, right). After calculating xᵢ for all evaluable epithelial states, the results were averaged, denoted x̅. Following this, each row of J was randomly permuted and the mean distance x̅′ was recalculated. This was repeated for a total of 100,000 iterations to calculate the empirical p-value of x. To create the UMAP plot in FIG. 20B, the following Seurat v4.0.1 commands were sequentially applied to the log-normalized scRNA-seq data of epithelial cell states from Ransick et al. FindVariableFeatures( ) with selection.method=“vst” and nfeatures=2000, ScaleData( ), RunPCA( ), FindNeighborso with dims=1:10, and RunUMAP( ) with dims=1:30.

### Application to Single-Cell ST Data

While the main goal of CytoSPACE is reconstruction of bulk ST data at the single-cell level, it is also directly applicable to single-cell ST data. To do this efficiently for extremely large single-cell ST datasets, a sampling routine was implemented to uniformly partition single-cell ST datasets without replacement into bins of up to 10,000 cells each (by default), which balances considerations of cellular diversity and mapping efficiency. Specifically, the single-cell ST dataset is first randomly partitioned without replacement into n bins of 10,000 ST cells each. Next, for each bin (1, . . . , n), 10,000 single-cell transcriptomes are sampled from the scRNA-seq query dataset (by default) according to the procedure described in “Harmonizing the number of cells per cell type-Duplication” above. While the entire procedure is reproducible and anchored to a specific seed at initialization, the scRNA-seq dataset is newly resampled for each bin 1, . . . , n in order to promote robustness. Finally, CytoSPACE is run on each bin and the results are combined to produce a single unified output.

For the analyses in FIGS. 21A and 21B and Extended Data FIGS. 22A to 22I, a preprocessed MERSCOPE profile of an FFPE human breast cancer sample (HumanBreastCancerPatientl) was downloaded from Vizgen (info.vizgen.com/merscope-ffpe-access). Cells with less than 100 transcripts and those with less than ten genes detected were excluded from the analysis, yielding 560,655 cells with 149 detected genes per cell, on average. The gene by cell count matrix was normalized by down-sampling, which eliminated potential confounding factors such as cell volume, by normalizing the total transcripts per cell to be the same (300 transcripts per cell). Using Seurat v4.1.1 to analyze the normalized data, the top 100 variable genes were identified using FindVariableFeatures( ) and the cells were clustered with FindClusters( ) using resolution=0.8 Leveraging canonical marker genes, clusters were annotated as fibroblasts (COL1A1 or COL5A1 high), endothelial cells (PECAM1 or VWF high), macrophages (FCGR3A or C1QC high), dendritic cells (CD1C or CD207 high), lymphocytes (CD3E, TRAC, ZAP70, MS4A1, GNLY, or MZB1 high), and epithelial (remaining). Lymphocytes were further clustered using the top 300 variable genes with resolution=1.2 and annotated as CD4 T cells (CD3E, TRAC, ZAP70, or FOXP3 high and no CD8A), CD8 T cells (CD3E, TRAC, or ZAP70 high and CD8A high), NK cells (GNLY high and no CD3E), B cells (MS4A1 high), and plasma cells (MZB1 high); clusters that did not meet these conditions but showed strong expressions of non-lymphocyte markers were annotated accordingly using epithelial and stromal markers above.

To account for errors in transcript assignment arising from overlapping cells in the z series, gene expression in the center z-plane (z=3) was compared with expression in the peripheral z-plane (z=0) for each segmented cell. Transcripts detected in either of the z-planes were first isolated as individual gene by cell count matrices. Then, all genes whose expression significantly differed between the two z-planes for one or more cell types were identified using a two-sided Wilcoxon test (nominal P<0.05). For each of these genes, if expression was significantly higher in the center z-plane for one cell type but significantly higher in the z=0 plane for another, the gene was considered a potential contaminant and set to zero in all cells of the latter cell type.

For the analysis presented in FIGS. 22A to 22E, the MERSCOPE dataset was randomly split (50:50) into “scRNA-seq” query and ST reference datasets (FIG. 22A). Then, query cells were mapped to the reference as described above, running CytoSPACE with 5 CPU cores, the number of cells per spot set to 1, and the global fractional abundance of each cell type set to its proportion in the reference dataset (FIG. 22B). Strong agreement was observed for cell type labels (FIG. 22C), and for each cell type, the gene expression profiles (GEPs) of mapped cells were more correlated with their assigned reference cells than with other reference cells of the same cell type (FIG. 22D). It was asked whether pairwise transcriptomic distances between single cells were retained (FIG. 22A). To do so for each evaluable cell type, the pairwise correlation matrix Q of single-cell GEPs (in log₂ space) in the scRNA-seq query dataset was calculated. This was done after assigning query cells to spatial locations in the reference. Then, the same was done for the reference dataset, yielding matrix R. Both matrices were ordered identically according to the same single-cell spatial coordinates, allowing determination of whether the spatial correlation structure was recapitulated among mapped cells. Indeed, by calculating a Retention index for each cell type, defined as the Pearson correlation between the two matrices, highly significant retention of pairwise distances was observed for each cell type (P<2.2e-16; FIG. 22E). To ensure a fair assessment, prior to creating each matrix, an equivalent number of cells per cell type were sampled (without replacement) based on the lowest common denominator in the reference dataset (n=150 cells). It was found that the degree of retention was proportional to the variance among GEPs in the reference dataset—that is, cell types with lower transcriptomic heterogeneity in the reference (i.e., more uniform GEPs) had less spatial structure and lower retention of pairwise distances, consistent with expectation (FIG. 22E).

As the MERSCOPE dataset lacked ESR1 (estrogen receptor) and PGR (progesterone receptor) among the 500 target genes but showed elevated expression of ERBB2 (encoding HER2), HER2+ breast tumors profiled by scRNA-seq were selected as the query dataset in FIGS. 21A and 21B (Tables 2). To ensure sufficient overlap in co-detected genes, cells from the scRNA-seq dataset with fewer than 50 expressed genes (CPM>0) overlapping the MERSCOPE panel were removed. Next, the scRNA-seq atlas was mapped to the MERSCOPE sample, running CytoSPACE with 5 CPU cores, the number of cells per spot set to 1, and the global fractional abundance of each cell type set to its proportion as determined above.

To evaluate the spatial enrichment of cell states in FIGS. 21A and 21B and FIGS. 22F to 22I, individual cells were first partitioned into two regions based on their Euclidean distance to epithelial cells. An epithelial cell was assigned to the tumor region if located within 100 μm of >50 epithelial cells. This threshold was selected based on a density-based analysis, where two major distributions of epithelial cell densities were observed, with ˜50 epithelial cells per radius of 100 μm representing a local minimum between the two distributions. Then, of the remaining cells, a cell was assigned to the tumor region if located within 100 μm of a tumor epithelial cell; otherwise, it was assigned to the adjacent normal region (i.e., stromal; FIG. 22H). For the analyses presented in FIG. 21B and FIG. 22I, the log₂ fold change of each gene in tumor vs. stromal regions was determined for CD4 and CD8 T cells with the raw MERSCOPE data (500 genes) or scRNA-seq data (whole transcriptome) mapped to MERSCOPE. Pre-ranked gene set enrichment analysis (GSEA) was applied as described in “Spatial enrichment analysis” for the top 200 signature genes of each pan-cancer T cell state defined by Zheng et al. except for ‘CD4T_IL7R-Tn,’ which lacked signature genes in the MERSCOPE dataset. For this analysis, fgsea package version 1.20.0 was used. Ground truth was determined as the rank of the log₂ fold change between the tumor odds ratio and normal odds ratio of each evaluated T cell state.

### Statistics

All statistical tests were two-sided unless stated otherwise. The Wilcoxon test was used to assess statistical differences between two groups. Adjustment for multiple hypothesis testing was done via Benjamini-Hochberg where applicable. Linear concordance was determined by Pearson (r) correlation or Spearman correlation (p), and a two-sided t test was used to assess whether the result was significantly non-zero. All statistical analyses were performed using R v3.5.1 and 4.0.2+, Python 3.8, MATLAB_R2019a, and Prism 9+(Graphpad Software, La Jolla, CA).

TABLE 1
Benchmarking results on simulated ST datasets.

| | Mean cells per spot | | | | | | | | | |
|---|---|---|---|---|---|---|---|---|---|---|
| | % of scRNA-seq | Single cell assignment precision across spot resolutions and scRNA-seq noise levels | | | | | | | | |
| | transcriptome perturbed | 5 | | | 15 | | | 30 | | |
| Method | Cell type | 5% | 10% | 25% | 5% | 10% | 25% | 5% | 10% | 25% |

a, Single cell assignment precision by cell type, mouse cerebellum

| | | | | | | | | | | |
|---|---|---|---|---|---|---|---|---|---|---|
| CytoSPACE | Astrocyte | 0.8208 | 0.8231 | 0.8042 | 0.7127 | 0.7345 | 0.6171 | 0.6804 | 0.6667 | 0.5493 |
| CytoSPACE | Bergmann | 0.8357 | 0.8260 | 0.8008 | 0.7162 | 0.6950 | 0.6087 | 0.5805 | 0.5285 | 0.4175 |
| CytoSPACE | Choroid | 0.7505 | 0.7303 | 0.7450 | 0.6239 | 0.6257 | 0.5743 | 0.4495 | 0.4459 | 0.3688 |
| CytoSPACE | Endothelial | 0.8682 | 0.8638 | 0.8492 | 0.7882 | 0.7848 | 0.7376 | 0.7174 | 0.7116 | 0.6354 |
| CytoSPACE | Fibroblast | 0.8343 | 0.8223 | 0.8042 | 0.7289 | 0.7309 | 0.6446 | 0.6165 | 0.5823 | 0.4950 |
| CytoSPACE | Granule | 0.8149 | 0.8037 | 0.7831 | 0.7300 | 0.7066 | 0.5927 | 0.5766 | 0.5123 | 0.3373 |
| CytoSPACE | Microglia | 0.8489 | 0.8489 | 0.8251 | 0.7199 | 0.7233 | 0.6027 | 0.5772 | 0.5501 | 0.3752 |
| CytoSPACE | Interneuron (Nnat+) | 0.8058 | 0.7780 | 0.7697 | 0.6466 | 0.6016 | 0.5782 | 0.4469 | 0.4464 | 0.3782 |
| CytoSPACE | Oligodendrocyte | 0.8687 | 0.8553 | 0.8327 | 0.7368 | 0.7184 | 0.6260 | 0.6224 | 0.5773 | 0.4404 |
| CytoSPACE | Purkinje | 0.7853 | 0.7756 | 0.7239 | 0.5545 | 0.5220 | 0.4180 | 0.4139 | 0.3539 | 0.2459 |
| CytoSPACE | Interneuron (Pvalb+) | 0.7531 | 0.7404 | 0.7675 | 0.6506 | 0.5709 | 0.6098 | 0.5477 | 0.4977 | 0.4362 |
| Tangram (all genes) | Astrocyte | 0.8282 | 0.7492 | 0.5759 | 0.7845 | 0.6551 | 0.4314 | 0.6804 | 0.5054 | 0.3803 |
| Tangram (all genes) | Bergmann | 0.6994 | 0.6038 | 0.4785 | 0.6164 | 0.5209 | 0.3712 | 0.4994 | 0.4149 | 0.2735 |
| Tangram (all genes) | Choroid | 0.6917 | 0.6073 | 0.4587 | 0.7174 | 0.5908 | 0.4220 | 0.6239 | 0.5101 | 0.3229 |
| Tangram (all genes) | Endothelial | 0.8843 | 0.8199 | 0.6471 | 0.9019 | 0.7716 | 0.5716 | 0.8272 | 0.7277 | 0.5095 |
| Tangram (all genes) | Fibroblast | 0.8163 | 0.7279 | 0.5673 | 0.7962 | 0.6737 | 0.5221 | 0.7490 | 0.6345 | 0.4337 |
| Tangram (all genes) | Granule | 0.7118 | 0.6035 | 0.4161 | 0.5396 | 0.4249 | 0.2667 | 0.3677 | 0.2677 | 0.1518 |
| Tangram (all genes) | Microglia | 0.7980 | 0.6927 | 0.4856 | 0.7674 | 0.6401 | 0.4584 | 0.6418 | 0.5365 | 0.3480 |
| Tangram (all genes) | Interneuron (Nnat+) | 0.7686 | 0.6791 | 0.4861 | 0.7284 | 0.5977 | 0.4422 | 0.5796 | 0.4740 | 0.3319 |
| Tangram (all genes) | Oligodendrocyte | 0.7495 | 0.6563 | 0.4834 | 0.6831 | 0.5688 | 0.4397 | 0.5476 | 0.4679 | 0.3225 |
| Tangram (all genes) | Purkinje | 0.5321 | 0.4528 | 0.2903 | 0.3603 | 0.2999 | 0.1969 | 0.2418 | 0.1969 | 0.1241 |
| Tangram (all genes) | Interneuron (Pvalb+) | 0.7692 | 0.6673 | 0.4752 | 0.6654 | 0.5787 | 0.4774 | 0.5560 | 0.4507 | 0.2819 |
| Tangram (marker genes) | Astrocyte | 0.5832 | 0.4967 | 0.3980 | 0.4199 | 0.2928 | 0.2086 | 0.2371 | 0.1183 | 0.0986 |
| Tangram (marker genes) | Bergmann | 0.5656 | 0.4771 | 0.3599 | 0.4578 | 0.3392 | 0.2327 | 0.3118 | 0.2259 | 0.1394 |
| Tangram (marker genes) | Choroid | 0.7450 | 0.6239 | 0.4587 | 0.6679 | 0.4936 | 0.3321 | 0.5523 | 0.4330 | 0.2422 |
| Tangram (marker genes) | Endothelial | 0.7862 | 0.7511 | 0.6018 | 0.7819 | 0.6867 | 0.5083 | 0.6574 | 0.5329 | 0.4129 |
| Tangram (marker genes) | Fibroblast | 0.7108 | 0.6667 | 0.5683 | 0.6205 | 0.5512 | 0.4598 | 0.5361 | 0.4900 | 0.3765 |
| Tangram (marker genes) | Granule | 0.2818 | 0.2291 | 0.1506 | 0.1218 | 0.0931 | 0.0543 | 0.0615 | 0.0450 | 0.0303 |
| Tangram (marker genes) | Microglia | 0.8319 | 0.7419 | 0.5823 | 0.7351 | 0.6248 | 0.4329 | 0.6435 | 0.5110 | 0.3531 |
| Tangram (marker genes) | Interneuron (Nnat+) | 0.6550 | 0.5297 | 0.3539 | 0.4310 | 0.3711 | 0.1939 | 0.3053 | 0.2215 | 0.1050 |
| Tangram (marker genes) | Oligodendrocyte | 0.6246 | 0.5674 | 0.4150 | 0.5004 | 0.4001 | 0.2886 | 0.2999 | 0.2406 | 0.1644 |
| Tangram (marker genes) | Purkinje | 0.4405 | 0.3810 | 0.2445 | 0.2463 | 0.1868 | 0.1255 | 0.1456 | 0.1140 | 0.0668 |
| Tangram (marker genes) | Interneuron (Pvalb+) | 0.5242 | 0.4113 | 0.2718 | 0.3086 | 0.2323 | 0.1498 | 0.1535 | 0.1127 | 0.0691 |
| CellTrek | Astrocyte | 0.0070 | 0.0069 | 0.0019 | 0.0028 | 0.0055 | 0.0040 | 0.0073 | 0.0026 | 0.0044 |
| CellTrek | Bergmann | 0.0047 | 0.0060 | 0.0052 | 0.0065 | 0.0054 | 0.0028 | 0.0080 | 0.0080 | 0.0069 |
| CellTrek | Choroid | 0.0216 | 0.0227 | 0.0177 | 0.0177 | 0.0172 | 0.0164 | 0.0202 | 0.0180 | 0.0231 |
| CellTrek | Endothelial | 0.0107 | 0.0066 | 0.0065 | 0.0080 | 0.0071 | 0.0039 | 0.0090 | 0.0062 | 0.0031 |
| CellTrek | Fibroblast | 0.0178 | 0.0170 | 0.0115 | 0.0148 | 0.0110 | 0.0097 | 0.0123 | 0.0124 | 0.0085 |
| CellTrek | Granule | 0.0033 | 0.0032 | 0.0026 | 0.0022 | 0.0022 | 0.0023 | 0.0030 | 0.0043 | 0.0033 |
| CellTrek | Microglia | 0.0134 | 0.0154 | 0.0112 | 0.0160 | 0.0160 | 0.0104 | 0.0189 | 0.0156 | 0.0117 |
| CellTrek | Interneuron (Nnat+) | 0.0118 | 0.0117 | 0.0070 | 0.0060 | 0.0087 | 0.0083 | 0.0094 | 0.0126 | 0.0117 |
| CellTrek | Oligodendrocyte | 0.0055 | 0.0055 | 0.0066 | 0.0049 | 0.0049 | 0.0024 | 0.0089 | 0.0081 | 0.0067 |
| CellTrek | Purkinje | 0.0114 | 0.0121 | 0.0079 | 0.0074 | 0.0075 | 0.0050 | 0.0106 | 0.0093 | 0.0085 |
| CellTrek | Interneuron (Pvalb+) | 0.0049 | 0.0065 | 0.0046 | 0.0041 | 0.0030 | 0.0045 | 0.0088 | 0.0071 | 0.0032 |
| DistMap | Astrocyte | 0.2530 | 0.2530 | 0.2331 | 0.0305 | 0.0358 | 0.0238 | 0.0119 | 0.0146 | 0.0093 |
| DistMap | Bergmann | 0.1292 | 0.1288 | 0.1201 | 0.0146 | 0.0126 | 0.0118 | 0.0055 | 0.0059 | 0.0047 |
| DistMap | Choroid | 0.1009 | 0.0789 | 0.0716 | 0.0294 | 0.0275 | 0.0220 | 0.0275 | 0.0165 | 0.0128 |
| DistMap | Endothelial | 0.1947 | 0.2064 | 0.1903 | 0.0337 | 0.0293 | 0.0234 | 0.0190 | 0.0190 | 0.0249 |
| DistMap | Fibroblast | 0.2239 | 0.2199 | 0.1928 | 0.0412 | 0.0432 | 0.0351 | 0.0201 | 0.0191 | 0.0171 |
| DistMap | Granule | 0.2297 | 0.2216 | 0.2047 | 0.0232 | 0.0236 | 0.0203 | 0.0065 | 0.0063 | 0.0062 |
| DistMap | Microglia | 0.1324 | 0.1256 | 0.1087 | 0.0730 | 0.0611 | 0.0441 | 0.0068 | 0.0102 | 0.0102 |
| DistMap | Interneuron (Nnat+) | 0.1775 | 0.1558 | 0.1519 | 0.0375 | 0.0276 | 0.0316 | 0.0079 | 0.0079 | 0.0079 |
| DistMap | Oligodendrocyte | 0.0685 | 0.0762 | 0.0790 | 0.0148 | 0.0191 | 0.0191 | 0.0021 | 0.0021 | 0.0092 |
| DistMap | Purkinje | 0.0728 | 0.0751 | 0.0632 | 0.0110 | 0.0105 | 0.0092 | 0.0055 | 0.0055 | 0.0060 |
| DistMap | Interneuron (Pvalb+) | 0.2545 | 0.2357 | 0.1933 | 0.0636 | 0.0457 | 0.0326 | 0.0196 | 0.0147 | 0.0106 |
| SpaOTsc | Astrocyte | 0.1762 | 0.1391 | 0.1457 | 0.0146 | 0.0238 | 0.0185 | 0.0066 | 0.0093 | 0.0093 |
| SpaOTsc | Bergmann | 0.1315 | 0.1197 | 0.1126 | 0.0181 | 0.0185 | 0.0193 | 0.0059 | 0.0059 | 0.0087 |
| SpaOTsc | Choroid | 0.0606 | 0.0606 | 0.0697 | 0.0422 | 0.0349 | 0.0312 | 0.0275 | 0.0275 | 0.0367 |
| SpaOTsc | Endothelial | 0.1171 | 0.0996 | 0.0952 | 0.0059 | 0.0073 | 0.0161 | 0.0044 | 0.0088 | 0.0102 |
| SpaOTsc | Fibroblast | 0.1325 | 0.1155 | 0.1396 | 0.0231 | 0.0241 | 0.0251 | 0.0141 | 0.0141 | 0.0151 |
| SpaOTsc | Granule | 0.2325 | 0.2125 | 0.1867 | 0.0417 | 0.0385 | 0.0325 | 0.0177 | 0.0176 | 0.0166 |
| SpaOTsc | Microglia | 0.0883 | 0.0679 | 0.0679 | 0.0374 | 0.0255 | 0.0255 | 0.0051 | 0.0051 | 0.0051 |
| SpaOTsc | Interneuron (Nnat+) | 0.1992 | 0.1874 | 0.2071 | 0.0592 | 0.0493 | 0.0493 | 0.0158 | 0.0158 | 0.0099 |
| SpaOTsc | Oligodendrocyte | 0.1108 | 0.1179 | 0.1186 | 0.0536 | 0.0501 | 0.0487 | 0.0318 | 0.0303 | 0.0303 |
| SpaOTsc | Purkinje | 0.0682 | 0.0691 | 0.0664 | 0.0169 | 0.0179 | 0.0179 | 0.0156 | 0.0128 | 0.0114 |
| SpaOTsc | Interneuron (Pvalb+) | 0.1452 | 0.1452 | 0.1395 | 0.0351 | 0.0277 | 0.0375 | 0.0228 | 0.0171 | 0.0188 |
| DEEPsc | Astrocyte | 0.0000 | 0.0000 | 0.0000 | 0.0000 | 0.0000 | 0.0000 | 0.0000 | 0.0000 | 0.0000 |
| DEEPsc | Bergmann | 0.0008 | 0.0008 | 0.0008 | 0.0012 | 0.0016 | 0.0016 | 0.0004 | 0.0004 | 0.0004 |
| DEEPsc | Choroid | 0.0018 | 0.0037 | 0.0018 | 0.0000 | 0.0000 | 0.0000 | 0.0037 | 0.0055 | 0.0037 |
| DEEPsc | Endothelial | 0.0015 | 0.0000 | 0.0015 | 0.0015 | 0.0015 | 0.0015 | 0.0000 | 0.0000 | 0.0000 |
| DEEPsc | Fibroblast | 0.0030 | 0.0020 | 0.0000 | 0.0030 | 0.0030 | 0.0020 | 0.0040 | 0.0050 | 0.0040 |
| DEEPsc | Granule | 0.0001 | 0.0003 | 0.0000 | 0.0002 | 0.0002 | 0.0003 | 0.0004 | 0.0004 | 0.0002 |
| DEEPsc | Microglia | 0.0000 | 0.0000 | 0.0000 | 0.0000 | 0.0000 | 0.0000 | 0.0017 | 0.0000 | 0.0000 |
| DEEPsc | Interneuron (Nnat+) | 0.0000 | 0.0000 | 0.0000 | 0.0000 | 0.0000 | 0.0000 | 0.0020 | 0.0020 | 0.0020 |
| DEEPsc | Oligodendrocyte | 0.0035 | 0.0014 | 0.0035 | 0.0000 | 0.0000 | 0.0021 | 0.0035 | 0.0028 | 0.0035 |
| DEEPsc | Purkinje | 0.0014 | 0.0009 | 0.0009 | 0.0014 | 0.0023 | 0.0027 | 0.0018 | 0.0014 | 0.0023 |
| DEEPsc | Interneuron (Pvalb+) | 0.0008 | 0.0008 | 0.0000 | 0.0000 | 0.0000 | 0.0000 | 0.0008 | 0.0000 | 0.0008 |
| SpaGE | Astrocyte | 0.0079 | 0.0079 | 0.0053 | 0.0013 | 0.0000 | 0.0026 | 0.0026 | 0.0026 | 0.0000 |
| SpaGE | Bergmann | 0.0087 | 0.0079 | 0.0071 | 0.0043 | 0.0051 | 0.0047 | 0.0071 | 0.0075 | 0.0083 |
| SpaGE | Choroid | 0.0147 | 0.0239 | 0.0220 | 0.0275 | 0.0220 | 0.0128 | 0.0220 | 0.0202 | 0.0275 |
| SpaGE | Endothelial | 0.0117 | 0.0117 | 0.0146 | 0.0073 | 0.0102 | 0.0102 | 0.0132 | 0.0073 | 0.0161 |
| SpaGE | Fibroblast | 0.0151 | 0.0131 | 0.0131 | 0.0120 | 0.0090 | 0.0120 | 0.0221 | 0.0191 | 0.0151 |
| SpaGE | Granule | 0.0082 | 0.0076 | 0.0061 | 0.0043 | 0.0033 | 0.0028 | 0.0040 | 0.0034 | 0.0039 |
| SpaGE | Microglia | 0.0221 | 0.0187 | 0.0119 | 0.0136 | 0.0119 | 0.0102 | 0.0119 | 0.0136 | 0.0204 |
| SpaGE | Interneuron (Nnat+) | 0.0118 | 0.0118 | 0.0158 | 0.0138 | 0.0138 | 0.0118 | 0.0138 | 0.0158 | 0.0158 |
| SpaGE | Oligodendrocyte | 0.0120 | 0.0120 | 0.0127 | 0.0120 | 0.0141 | 0.0092 | 0.0106 | 0.0148 | 0.0155 |
| SpaGE | Purkinje | 0.0137 | 0.0137 | 0.0147 | 0.0133 | 0.0119 | 0.0105 | 0.0151 | 0.0114 | 0.0137 |
| SpaGE | Interneuron (Pvalb+) | 0.0098 | 0.0106 | 0.0131 | 0.0073 | 0.0073 | 0.0082 | 0.0098 | 0.0065 | 0.0106 |
| Seurat | Astrocyte | 0.0040 | 0.0040 | 0.0013 | 0.0040 | 0.0093 | 0.0040 | 0.0000 | 0.0000 | 0.0000 |
| Seurat | Bergmann | 0.0055 | 0.0067 | 0.0051 | 0.0012 | 0.0016 | 0.0035 | 0.0004 | 0.0012 | 0.0008 |
| Seurat | Choroid | 0.0183 | 0.0165 | 0.0128 | 0.0110 | 0.0202 | 0.0073 | 0.0165 | 0.0092 | 0.0202 |
| Seurat | Endothelial | 0.0102 | 0.0088 | 0.0102 | 0.0102 | 0.0015 | 0.0015 | 0.0015 | 0.0029 | 0.0044 |
| Seurat | Fibroblast | 0.0281 | 0.0231 | 0.0241 | 0.0090 | 0.0090 | 0.0090 | 0.0080 | 0.0100 | 0.0110 |
| Seurat | Granule | 0.0009 | 0.0014 | 0.0013 | 0.0008 | 0.0020 | 0.0008 | 0.0004 | 0.0019 | 0.0022 |
| Seurat | Microglia | 0.0170 | 0.0238 | 0.0187 | 0.0102 | 0.0170 | 0.0102 | 0.0187 | 0.0170 | 0.0085 |
| Seurat | Interneuron (Nnat+) | 0.0059 | 0.0099 | 0.0059 | 0.0059 | 0.0079 | 0.0079 | 0.0039 | 0.0039 | 0.0020 |
| Seurat | Oligodendrocyte | 0.0014 | 0.0007 | 0.0014 | 0.0042 | 0.0035 | 0.0028 | 0.0035 | 0.0064 | 0.0042 |
| Seurat | Purkinje | 0.0064 | 0.0064 | 0.0060 | 0.0046 | 0.0041 | 0.0050 | 0.0101 | 0.0128 | 0.0092 |
| Seurat | Interneuron (Pvalb+) | 0.0033 | 0.0057 | 0.0041 | 0.0000 | 0.0057 | 0.0008 | 0.0000 | 0.0024 | 0.0057 |
| Harmony | Astrocyte | 0.0278 | 0.0185 | 0.0000 | 0.0026 | 0.0013 | 0.0000 | 0.0013 | 0.0000 | 0.0000 |
| Harmony | Bergmann | 0.0217 | 0.0071 | 0.0004 | 0.0035 | 0.0024 | 0.0000 | 0.0024 | 0.0016 | 0.0000 |
| Harmony | Choroid | 0.0532 | 0.0349 | 0.0147 | 0.0147 | 0.0128 | 0.0110 | 0.0183 | 0.0128 | 0.0055 |
| Harmony | Endothelial | 0.0498 | 0.0293 | 0.0015 | 0.0059 | 0.0029 | 0.0029 | 0.0015 | 0.0015 | 0.0000 |
| Harmony | Fibroblast | 0.0683 | 0.0552 | 0.0191 | 0.0171 | 0.0090 | 0.0060 | 0.0080 | 0.0040 | 0.0020 |
| Harmony | Granule | 0.0086 | 0.0044 | 0.0004 | 0.0014 | 0.0011 | 0.0002 | 0.0008 | 0.0005 | 0.0003 |
| Harmony | Microglia | 0.0424 | 0.0289 | 0.0068 | 0.0051 | 0.0051 | 0.0017 | 0.0136 | 0.0034 | 0.0034 |
| Harmony | Interneuron (Nnat+) | 0.0256 | 0.0217 | 0.0000 | 0.0059 | 0.0039 | 0.0000 | 0.0059 | 0.0020 | 0.0000 |
| Harmony | Oligodendrocyte | 0.0261 | 0.0106 | 0.0007 | 0.0049 | 0.0021 | 0.0007 | 0.0049 | 0.0035 | 0.0000 |
| Harmony | Purkinje | 0.0321 | 0.0169 | 0.0005 | 0.0060 | 0.0037 | 0.0000 | 0.0037 | 0.0032 | 0.0000 |
| Harmony | Interneuron (Pvalb+) | 0.0269 | 0.0147 | 0.0000 | 0.0033 | 0.0008 | 0.0000 | 0.0008 | 0.0000 | 0.0000 |
| LIGER | Astrocyte | 0.0013 | 0.0053 | 0.0040 | 0.0026 | 0.0013 | 0.0026 | 0.0026 | 0.0013 | 0.0000 |
| LIGER | Bergmann | 0.0028 | 0.0039 | 0.0020 | 0.0063 | 0.0087 | 0.0032 | 0.0059 | 0.0043 | 0.0059 |
| LIGER | Choroid | 0.0294 | 0.0257 | 0.0073 | 0.0073 | 0.0165 | 0.0092 | 0.0404 | 0.0128 | 0.0165 |
| LIGER | Endothelial | 0.0073 | 0.0073 | 0.0029 | 0.0088 | 0.0088 | 0.0073 | 0.0073 | 0.0059 | 0.0015 |
| LIGER | Fibroblast | 0.0120 | 0.0131 | 0.0070 | 0.0110 | 0.0120 | 0.0110 | 0.0090 | 0.0050 | 0.0040 |
| LIGER | Granule | 0.0023 | 0.0020 | 0.0011 | 0.0023 | 0.0021 | 0.0020 | 0.0047 | 0.0053 | 0.0030 |
| LIGER | Microglia | 0.0136 | 0.0136 | 0.0068 | 0.0119 | 0.0051 | 0.0051 | 0.0068 | 0.0119 | 0.0034 |
| LIGER | Interneuron (Nnat+) | 0.0039 | 0.0059 | 0.0039 | 0.0079 | 0.0059 | 0.0020 | 0.0079 | 0.0020 | 0.0020 |
| LIGER | Oligodendrocyte | 0.0021 | 0.0042 | 0.0035 | 0.0042 | 0.0021 | 0.0035 | 0.0064 | 0.0071 | 0.0106 |
| LIGER | Purkinje | 0.0092 | 0.0050 | 0.0032 | 0.0060 | 0.0082 | 0.0032 | 0.0046 | 0.0087 | 0.0082 |
| LIGER | Interneuron (Pvalb+) | 0.0106 | 0.0049 | 0.0098 | 0.0024 | 0.0057 | 0.0033 | 0.0065 | 0.0033 | 0.0033 |
| Pearson correlation | Astrocyte | 0.6053 | 0.5974 | 0.5881 | 0.6132 | 0.5788 | 0.5470 | 0.5245 | 0.5007 | 0.4079 |
| Pearson correlation | Bergmann | 0.4356 | 0.4313 | 0.4238 | 0.4147 | 0.4053 | 0.3816 | 0.3454 | 0.3301 | 0.2753 |
| Pearson correlation | Choroid | 0.2752 | 0.2697 | 0.2661 | 0.2147 | 0.2110 | 0.1982 | 0.1835 | 0.1798 | 0.1706 |
| Pearson correlation | Endothelial | 0.6340 | 0.6296 | 0.6384 | 0.6047 | 0.5915 | 0.5681 | 0.5520 | 0.5344 | 0.5066 |
| Pearson correlation | Fibroblast | 0.4709 | 0.4699 | 0.4518 | 0.4247 | 0.4187 | 0.3916 | 0.3805 | 0.3624 | 0.3474 |
| Pearson correlation | Granule | 0.6298 | 0.6279 | 0.6179 | 0.5916 | 0.5718 | 0.4998 | 0.4714 | 0.4133 | 0.2807 |
| Pearson correlation | Microglia | 0.2954 | 0.3022 | 0.2920 | 0.3633 | 0.3752 | 0.3413 | 0.3158 | 0.3277 | 0.2581 |
| Pearson correlation | Interneuron (Nnat+) | 0.4615 | 0.4497 | 0.4675 | 0.4320 | 0.4241 | 0.4241 | 0.3807 | 0.3432 | 0.3353 |
| Pearson correlation | Oligodendrocyte | 0.4488 | 0.4446 | 0.4474 | 0.4510 | 0.4375 | 0.4178 | 0.3881 | 0.3585 | 0.3155 |
| Pearson correlation | Purkinje | 0.1685 | 0.1703 | 0.1685 | 0.1754 | 0.1726 | 0.1571 | 0.1415 | 0.1346 | 0.1117 |
| Pearson correlation | Interneuron (Pvalb+) | 0.5351 | 0.5253 | 0.5261 | 0.5082 | 0.5008 | 0.4804 | 0.4372 | 0.4339 | 0.3825 |
| Spearman correlation | Astrocyte | 0.5960 | 0.5947 | 0.5841 | 0.5907 | 0.5603 | 0.5377 | 0.4940 | 0.4623 | 0.3762 |
| Spearman correlation | Bergmann | 0.4325 | 0.4285 | 0.4187 | 0.4053 | 0.3946 | 0.3726 | 0.3470 | 0.3277 | 0.2690 |
| Spearman correlation | Choroid | 0.2679 | 0.2606 | 0.2642 | 0.1963 | 0.1908 | 0.1908 | 0.1835 | 0.1743 | 0.1688 |
| Spearman correlation | Endothelial | 0.6252 | 0.6266 | 0.6252 | 0.5871 | 0.5769 | 0.5608 | 0.5388 | 0.5242 | 0.5022 |
| Spearman correlation | Fibroblast | 0.4598 | 0.4629 | 0.4478 | 0.4127 | 0.4116 | 0.3815 | 0.3655 | 0.3584 | 0.3333 |
| Spearman correlation | Granule | 0.6162 | 0.6153 | 0.6031 | 0.5700 | 0.5518 | 0.4692 | 0.4098 | 0.3541 | 0.2250 |
| Spearman correlation | Microglia | 0.2937 | 0.2937 | 0.2852 | 0.3616 | 0.3599 | 0.3294 | 0.2971 | 0.3124 | 0.2564 |
| Spearman correlation | Interneuron (Nnat+) | 0.4517 | 0.4438 | 0.4517 | 0.4201 | 0.4083 | 0.4083 | 0.3609 | 0.3274 | 0.3116 |
| Spearman correlation | Oligodendrocyte | 0.4474 | 0.4404 | 0.4375 | 0.4545 | 0.4397 | 0.4171 | 0.4072 | 0.3754 | 0.3197 |
| Spearman correlation | Purkinje | 0.1648 | 0.1671 | 0.1667 | 0.1722 | 0.1671 | 0.1461 | 0.1300 | 0.1200 | 0.1035 |
| Spearman correlation | Interneuron (Pvalb+) | 0.5294 | 0.5179 | 0.5237 | 0.4976 | 0.4861 | 0.4657 | 0.4201 | 0.4119 | 0.3540 |
| Euclidean distance | Astrocyte | 0.1007 | 0.0993 | 0.1139 | 0.0066 | 0.0066 | 0.0079 | 0.0013 | 0.0013 | 0.0013 |
| Euclidean distance | Bergmann | 0.1122 | 0.1107 | 0.1426 | 0.0079 | 0.0079 | 0.0142 | 0.0032 | 0.0032 | 0.0043 |
| Euclidean distance | Choroid | 0.1083 | 0.1046 | 0.1303 | 0.0147 | 0.0147 | 0.0330 | 0.0037 | 0.0037 | 0.0128 |
| Euclidean distance | Endothelial | 0.1991 | 0.1962 | 0.2372 | 0.0088 | 0.0088 | 0.0161 | 0.0015 | 0.0015 | 0.0029 |
| Euclidean distance | Fibroblast | 0.1506 | 0.1506 | 0.1867 | 0.0201 | 0.0221 | 0.0311 | 0.0070 | 0.0070 | 0.0090 |
| Euclidean distance | Granule | 0.0886 | 0.0841 | 0.0976 | 0.0070 | 0.0067 | 0.0077 | 0.0031 | 0.0031 | 0.0032 |
| Euclidean distance | Microglia | 0.0917 | 0.0951 | 0.0968 | 0.0051 | 0.0051 | 0.0068 | 0.0034 | 0.0034 | 0.0034 |
| Euclidean distance | Interneuron (Nnat+) | 0.1854 | 0.1933 | 0.2268 | 0.0138 | 0.0138 | 0.0592 | 0.0059 | 0.0059 | 0.0099 |
| Euclidean distance | Oligodendrocyte | 0.1489 | 0.1524 | 0.1976 | 0.0042 | 0.0042 | 0.0141 | 0.0007 | 0.0007 | 0.0007 |
| Euclidean distance | Purkinje | 0.0650 | 0.0636 | 0.0691 | 0.0069 | 0.0064 | 0.0073 | 0.0041 | 0.0041 | 0.0046 |
| Euclidean distance | Interneuron (Pvalb+) | 0.1550 | 0.1525 | 0.1982 | 0.0073 | 0.0073 | 0.0147 | 0.0024 | 0.0024 | 0.0033 |

b, Single cell assignment precision by cell type, mouse hippocampus

| | | | | | | | | | | |
|---|---|---|---|---|---|---|---|---|---|---|
| CytoSPACE | Astrocyte | 0.7917 | 0.7723 | 0.7437 | 0.7052 | 0.6764 | 0.5375 | 0.4881 | 0.4165 | 0.2851 |
| CytoSPACE | CA1 | 0.7889 | 0.7815 | 0.8000 | 0.7185 | 0.7148 | 0.6741 | 0.6630 | 0.6111 | 0.5667 |
| CytoSPACE | CA2/CA3 | 0.9310 | 0.9425 | 0.8837 | 0.8276 | 0.8161 | 0.7011 | 0.6667 | 0.5977 | 0.5057 |
| CytoSPACE | Cajal-Retzius | 0.6642 | 0.6622 | 0.6232 | 0.4456 | 0.4149 | 0.3178 | 0.2932 | 0.2420 | 0.1620 |
| CytoSPACE | Choroid | 0.6775 | 0.6630 | 0.6377 | 0.4565 | 0.4529 | 0.4293 | 0.3641 | 0.3551 | 0.3207 |
| CytoSPACE | Dentate | 0.7321 | 0.7100 | 0.6996 | 0.5958 | 0.5673 | 0.5002 | 0.4097 | 0.3676 | 0.2925 |
| CytoSPACE | Endothelia | 0.9408 | 0.9326 | 0.9161 | 0.8849 | 0.8569 | 0.7418 | 0.7270 | 0.6628 | 0.5082 |
| CytoSPACE | Subiculum Entorhinal (Nxph3+) | 0.8221 | 0.8104 | 0.7786 | 0.7414 | 0.7145 | 0.6159 | 0.5967 | 0.5416 | 0.4069 |
| CytoSPACE | Ependymal | 0.8862 | 0.8806 | 0.8646 | 0.7600 | 0.7446 | 0.6883 | 0.5925 | 0.5529 | 0.4663 |
| CytoSPACE | Fibroblast | 0.8951 | 0.8728 | 0.8504 | 0.8147 | 0.7813 | 0.7210 | 0.6272 | 0.5915 | 0.4488 |
| CytoSPACE | Interneuron (Gad2+) | 0.8525 | 0.8351 | 0.8243 | 0.7592 | 0.7202 | 0.6529 | 0.6226 | 0.6052 | 0.5011 |
| CytoSPACE | Microglia | 0.8706 | 0.8678 | 0.8316 | 0.8053 | 0.7576 | 0.6212 | 0.6013 | 0.5153 | 0.3355 |
| CytoSPACE | Mural | 1.0000 | 1.0000 | 0.9250 | 0.9500 | 0.8875 | 0.7625 | 0.6000 | 0.5000 | NA |
| CytoSPACE | Neurogenesis | 0.7497 | 0.7344 | 0.6971 | 0.6058 | 0.5455 | 0.4652 | 0.4461 | 0.3608 | 0.2841 |
| CytoSPACE | Oligodendrocyte | 0.9171 | 0.8912 | 0.8182 | 0.8187 | 0.7306 | 0.5597 | 0.5000 | NA | 0.4000 |
| CytoSPACE | Polydendrocyte | 0.8344 | 0.8168 | 0.7949 | 0.6459 | 0.6170 | 0.5031 | 0.4619 | 0.4198 | 0.2857 |
| CytoSPACE | Subiculum (Slc17a6+) | 0.8085 | 0.7904 | 0.7929 | 0.7023 | 0.6731 | 0.5824 | 0.5409 | 0.4861 | 0.3712 |
| Tangram (all genes) | Astrocyte | 0.5593 | 0.4493 | 0.3115 | 0.3474 | 0.2692 | 0.1775 | 0.2099 | 0.1498 | 0.1037 |
| Tangram (all genes) | CA1 | 0.8111 | 0.7296 | 0.6111 | 0.7000 | 0.6556 | 0.5481 | 0.6296 | 0.5519 | 0.5000 |
| Tangram (all genes) | CA2/CA3 | 0.6322 | 0.5862 | 0.4767 | 0.5057 | 0.4598 | 0.3678 | 0.4368 | 0.3793 | 0.3333 |
| Tangram (all genes) | Cajal-Retzius | 0.4386 | 0.3635 | 0.2495 | 0.2329 | 0.1823 | 0.1113 | 0.1224 | 0.0949 | 0.0677 |
| Tangram (all genes) | Choroid | 0.5598 | 0.5362 | 0.4493 | 0.5163 | 0.4547 | 0.3587 | 0.3967 | 0.3315 | 0.3025 |
| Tangram (all genes) | Dentate | 0.6450 | 0.5528 | 0.4200 | 0.4923 | 0.4250 | 0.3048 | 0.3631 | 0.3098 | 0.2047 |
| Tangram (all genes) | Endothelia | 0.8487 | 0.7829 | 0.6118 | 0.7467 | 0.6201 | 0.4128 | 0.5674 | 0.4342 | 0.2961 |
| Tangram (all genes) | Subiculum Entorhinal (Nxph3+) | 0.7285 | 0.6527 | 0.5406 | 0.6274 | 0.5403 | 0.4063 | 0.4853 | 0.4251 | 0.3137 |
| Tangram (all genes) | Ependymal | 0.8386 | 0.7502 | 0.5962 | 0.7143 | 0.6122 | 0.4447 | 0.5553 | 0.4595 | 0.3296 |
| Tangram (all genes) | Fibroblast | 0.8147 | 0.7277 | 0.5781 | 0.7165 | 0.5714 | 0.4129 | 0.5603 | 0.4353 | 0.3140 |
| Tangram (all genes) | Interneuron (Gad2+) | 0.8308 | 0.7462 | 0.5944 | 0.7093 | 0.6269 | 0.5510 | 0.6161 | 0.5922 | 0.4534 |
| Tangram (all genes) | Microglia | 0.7491 | 0.6397 | 0.4549 | 0.5792 | 0.4627 | 0.2935 | 0.4065 | 0.3035 | 0.1777 |
| Tangram (all genes) | Mural | 0.8875 | 0.9250 | 0.7750 | 0.8750 | 0.8000 | 0.6500 | 0.8000 | 0.1667 | NA |
| Tangram (all genes) | Neurogenesis | 0.5888 | 0.4829 | 0.3771 | 0.4115 | 0.3049 | 0.1978 | 0.2303 | 0.1698 | 0.1402 |
| Tangram (all genes) | Oligodendrocyte | 0.5648 | 0.5130 | 0.3636 | 0.3627 | 0.2435 | 0.1698 | 0.2143 | NA | 0.0667 |
| Tangram (all genes) | Polydendrocyte | 0.6301 | 0.5600 | 0.4408 | 0.4926 | 0.4137 | 0.3313 | 0.3699 | 0.3050 | 0.2147 |
| Tangram (all genes) | Subiculum (Slc17a6+) | 0.6986 | 0.6366 | 0.5029 | 0.5813 | 0.5055 | 0.3386 | 0.4598 | 0.3615 | 0.2618 |
| Tangram (marker genes) | Astrocyte | 0.4005 | 0.3182 | 0.2057 | 0.2412 | 0.1623 | 0.1116 | 0.1502 | 0.0889 | 0.0503 |
| Tangram (marker genes) | CA1 | 0.5370 | 0.5148 | 0.3926 | 0.4111 | 0.3593 | 0.2889 | 0.2704 | 0.2852 | 0.1852 |
| Tangram (marker genes) | CA2/CA3 | 0.4023 | 0.3103 | 0.3023 | 0.3448 | 0.3448 | 0.2414 | 0.2759 | 0.2759 | 0.2299 |
| Tangram (marker genes) | Cajal-Retzius | 0.2199 | 0.1623 | 0.0965 | 0.1040 | 0.0598 | 0.0256 | 0.0447 | 0.0328 | 0.0224 |
| Tangram (marker genes) | Choroid | 0.5217 | 0.5163 | 0.4221 | 0.3170 | 0.3043 | 0.2790 | 0.2210 | 0.2156 | 0.1812 |
| Tangram (marker genes) | Dentate | 0.2962 | 0.2556 | 0.1664 | 0.1700 | 0.1400 | 0.0945 | 0.1050 | 0.0847 | 0.0526 |
| Tangram (marker genes) | Endothelia | 0.8421 | 0.7336 | 0.5592 | 0.7911 | 0.6283 | 0.4359 | 0.6036 | 0.5197 | 0.3224 |
| Tangram (marker genes) | Subiculum Entorhinal (Nxph3+) | 0.3236 | 0.2987 | 0.2214 | 0.2241 | 0.1613 | 0.1111 | 0.1306 | 0.0935 | 0.0817 |
| Tangram (marker genes) | Ependymal | 0.5374 | 0.4496 | 0.3055 | 0.3723 | 0.2876 | 0.1688 | 0.2208 | 0.1626 | 0.0940 |
| Tangram (marker genes) | Fibroblast | 0.8371 | 0.7433 | 0.5737 | 0.7478 | 0.5893 | 0.4018 | 0.5156 | 0.3438 | 0.1953 |
| Tangram (marker genes) | Interneuron (Gad2+) | 0.5597 | 0.5011 | 0.4555 | 0.4490 | 0.3818 | 0.3905 | 0.3167 | 0.3167 | 0.2798 |
| Tangram (marker genes) | Microglia | 0.7292 | 0.6240 | 0.4520 | 0.6091 | 0.5039 | 0.3163 | 0.4698 | 0.3475 | 0.1834 |
| Tangram (marker genes) | Mural | 0.8750 | 0.9375 | 0.7875 | 0.8625 | 0.8500 | 0.7750 | 0.2000 | 0.2500 | NA |
| Tangram (marker genes) | Neurogenesis | 0.2821 | 0.2100 | 0.1445 | 0.1308 | 0.0871 | 0.0578 | 0.0812 | 0.0462 | 0.0390 |
| Tangram (marker genes) | Oligodendrocyte | 0.3679 | 0.3264 | 0.2460 | 0.1347 | 0.1347 | 0.0566 | 0.0000 | NA | 0.0000 |
| Tangram (marker genes) | Polydendrocyte | 0.5162 | 0.4592 | 0.3514 | 0.3611 | 0.2883 | 0.2279 | 0.2638 | 0.2191 | 0.1481 |
| Tangram (marker genes) | Subiculum (Slc17a6+) | 0.3339 | 0.2689 | 0.2071 | 0.2090 | 0.1461 | 0.0831 | 0.1136 | 0.0637 | 0.0512 |
| CellTrek | Astrocyte | 0.0023 | 0.0020 | 0.0038 | 0.0052 | 0.0031 | 0.0014 | 0.0017 | 0.0044 | 0.0035 |
| CellTrek | CA1 | 0.0155 | 0.0163 | 0.0229 | 0.0188 | 0.0184 | 0.0186 | 0.0158 | 0.0146 | 0.0191 |
| CellTrek | CA2/CA3 | 0.0200 | 0.0127 | 0.0163 | 0.0023 | 0.0071 | 0.0139 | 0.0000 | 0.0000 | 0.0000 |
| CellTrek | Cajal-Retzius | 0.0085 | 0.0049 | 0.0043 | 0.0073 | 0.0052 | 0.0038 | 0.0056 | 0.0035 | 0.0034 |
| CellTrek | Choroid | 0.0123 | 0.0090 | 0.0097 | 0.0120 | 0.0154 | 0.0120 | 0.0193 | 0.0136 | 0.0207 |
| CellTrek | Dentate | 0.0095 | 0.0098 | 0.0066 | 0.0090 | 0.0062 | 0.0054 | 0.0074 | 0.0064 | 0.0062 |
| CellTrek | Endothelia | 0.0018 | 0.0021 | 0.0039 | 0.0043 | 0.0047 | 0.0032 | 0.0087 | 0.0083 | 0.0050 |
| CellTrek | Subiculum Entorhinal (Nxph3+) | 0.0054 | 0.0050 | 0.0056 | 0.0052 | 0.0085 | 0.0082 | 0.0071 | 0.0038 | 0.0049 |
| CellTrek | Ependymal | 0.0064 | 0.0040 | 0.0034 | 0.0024 | 0.0042 | 0.0019 | 0.0045 | 0.0054 | 0.0052 |
| CellTrek | Fibroblast | 0.0151 | 0.0092 | 0.0050 | 0.0093 | 0.0145 | 0.0127 | 0.0130 | 0.0226 | 0.0112 |
| CellTrek | Interneuron (Gad2+) | 0.0175 | 0.0119 | 0.0237 | 0.0085 | 0.0136 | 0.0109 | 0.0130 | 0.0119 | 0.0097 |
| CellTrek | Microglia | 0.0127 | 0.0119 | 0.0059 | 0.0082 | 0.0110 | 0.0091 | 0.0059 | 0.0060 | 0.0054 |
| CellTrek | Mural | 0.0335 | 0.0158 | 0.0242 | 0.0284 | 0.0184 | 0.0327 | 0.0156 | 0.0083 | 0.0272 |
| CellTrek | Neurogenesis | 0.0169 | 0.0152 | 0.0111 | 0.0069 | 0.0078 | 0.0058 | 0.0067 | 0.0056 | 0.0081 |
| CellTrek | Oligodendrocyte | 0.0052 | 0.0071 | 0.0066 | 0.0063 | 0.0000 | 0.0057 | 0.0166 | 0.0139 | 0.0096 |
| CellTrek | Polydendrocyte | 0.0241 | 0.0190 | 0.0180 | 0.0162 | 0.0142 | 0.0114 | 0.0138 | 0.0193 | 0.0182 |
| CellTrek | Subiculum (Slc17a6+) | 0.0066 | 0.0096 | 0.0076 | 0.0048 | 0.0077 | 0.0069 | 0.0070 | 0.0091 | 0.0089 |
| DistMap | Astrocyte | 0.1451 | 0.1292 | 0.0898 | 0.0148 | 0.0135 | 0.0117 | 0.0074 | 0.0086 | 0.0074 |
| DistMap | CA1 | 0.1630 | 0.1481 | 0.1148 | 0.0148 | 0.0148 | 0.0148 | 0.0148 | 0.0111 | 0.0111 |
| DistMap | CA2/CA3 | 0.1954 | 0.1724 | 0.1494 | 0.0460 | 0.0460 | 0.0115 | 0.0115 | 0.0230 | 0.0000 |
| DistMap | Cajal-Retzius | 0.0953 | 0.0929 | 0.0748 | 0.0240 | 0.0238 | 0.0186 | 0.0133 | 0.0134 | 0.0096 |
| DistMap | Choroid | 0.0362 | 0.0344 | 0.0308 | 0.0109 | 0.0109 | 0.0109 | 0.0109 | 0.0109 | 0.0109 |
| DistMap | Dentate | 0.1234 | 0.1209 | 0.0755 | 0.0184 | 0.0184 | 0.0110 | 0.0106 | 0.0103 | 0.0074 |
| DistMap | Endothelia | 0.2878 | 0.2961 | 0.2007 | 0.0296 | 0.0362 | 0.0296 | 0.0214 | 0.0181 | 0.0477 |
| DistMap | Subiculum Entorhinal (Nxph3+) | 0.2100 | 0.1780 | 0.0909 | 0.0307 | 0.0294 | 0.0205 | 0.0230 | 0.0243 | 0.0166 |
| DistMap | Ependymal | 0.2474 | 0.2443 | 0.1490 | 0.0315 | 0.0346 | 0.0192 | 0.0155 | 0.0173 | 0.0099 |
| DistMap | Fibroblast | 0.3549 | 0.3393 | 0.2433 | 0.0558 | 0.0625 | 0.0424 | 0.0134 | 0.0201 | 0.0179 |
| DistMap | Interneuron (Gad2+) | 0.2755 | 0.2603 | 0.1887 | 0.0325 | 0.0347 | 0.0304 | 0.0195 | 0.0217 | 0.0260 |
| DistMap | Microglia | 0.2104 | 0.2168 | 0.1493 | 0.0277 | 0.0284 | 0.0206 | 0.0156 | 0.0171 | 0.0121 |
| DistMap | Mural | 0.4125 | 0.4750 | 0.4000 | 0.1000 | 0.0875 | 0.1250 | 0.0500 | 0.0500 | 0.1375 |
| DistMap | Neurogenesis | 0.1527 | 0.1404 | 0.0948 | 0.0288 | 0.0281 | 0.0169 | 0.0193 | 0.0172 | 0.0088 |
| DistMap | Oligodendrocyte | 0.2021 | 0.2228 | 0.1399 | 0.0259 | 0.0259 | 0.0207 | 0.0104 | 0.0104 | 0.0052 |
| DistMap | Polydendrocyte | 0.1613 | 0.1604 | 0.1192 | 0.0219 | 0.0193 | 0.0140 | 0.0114 | 0.0096 | 0.0061 |
| DistMap | Subiculum (Slc17a6+) | 0.1697 | 0.1510 | 0.0907 | 0.0298 | 0.0305 | 0.0194 | 0.0194 | 0.0208 | 0.0132 |
| DEEPsc | Astrocyte | 0.0000 | 0.0000 | 0.0000 | 0.0000 | 0.0000 | 0.0000 | 0.0000 | 0.0000 | 0.0000 |
| DEEPsc | CA1 | 0.0000 | 0.0074 | 0.0037 | 0.0000 | 0.0000 | 0.0000 | 0.0000 | 0.0000 | 0.0000 |
| DEEPsc | CA2/CA3 | 0.0230 | 0.0230 | 0.0115 | 0.0000 | 0.0000 | 0.0000 | 0.0000 | 0.0000 | 0.0000 |
| DEEPsc | Cajal-Retzius | 0.0007 | 0.0001 | 0.0004 | 0.0009 | 0.0006 | 0.0006 | 0.0011 | 0.0007 | 0.0010 |
| DEEPsc | Choroid | 0.0018 | 0.0018 | 0.0018 | 0.0036 | 0.0036 | 0.0018 | 0.0145 | 0.0054 | 0.0018 |
| DEEPsc | Dentate | 0.0004 | 0.0000 | 0.0000 | 0.0007 | 0.0004 | 0.0007 | 0.0018 | 0.0000 | 0.0004 |
| DEEPsc | Endothelia | 0.0000 | 0.0000 | 0.0000 | 0.0000 | 0.0000 | 0.0000 | 0.0000 | 0.0000 | 0.0000 |
| DEEPsc | Subiculum Entorhinal (Nxph3+) | 0.0000 | 0.0000 | 0.0000 | 0.0013 | 0.0013 | 0.0013 | 0.0000 | 0.0000 | 0.0000 |
| DEEPsc | Ependymal | 0.0019 | 0.0012 | 0.0019 | 0.0019 | 0.0000 | 0.0000 | 0.0049 | 0.0031 | 0.0019 |
| DEEPsc | Fibroblast | 0.0000 | 0.0000 | 0.0000 | 0.0000 | 0.0000 | 0.0000 | 0.0000 | 0.0000 | 0.0022 |
| DEEPsc | Interneuron (Gad2+) | 0.0000 | 0.0000 | 0.0000 | 0.0022 | 0.0022 | 0.0022 | 0.0022 | 0.0022 | 0.0022 |
| DEEPsc | Microglia | 0.0000 | 0.0000 | 0.0007 | 0.0028 | 0.0028 | 0.0028 | 0.0014 | 0.0007 | 0.0007 |
| DEEPsc | Mural | 0.0000 | 0.0000 | 0.0000 | 0.0000 | 0.0000 | 0.0000 | 0.0000 | 0.0000 | 0.0000 |
| DEEPsc | Neurogenesis | 0.0000 | 0.0000 | 0.0004 | 0.0000 | 0.0000 | 0.0000 | 0.0000 | 0.0004 | 0.0000 |
| DEEPsc | Oligodendrocyte | 0.0000 | 0.0000 | 0.0000 | 0.0052 | 0.0052 | 0.0000 | 0.0000 | 0.0000 | 0.0000 |
| DEEPsc | Polydendrocyte | 0.0009 | 0.0009 | 0.0000 | 0.0018 | 0.0018 | 0.0018 | 0.0009 | 0.0009 | 0.0018 |
| DEEPsc | Subiculum (Slc17a6+) | 0.0000 | 0.0007 | 0.0007 | 0.0007 | 0.0021 | 0.0007 | 0.0014 | 0.0021 | 0.0014 |
| SpaOTsc | Astrocyte | 0.1261 | 0.1162 | 0.1162 | 0.0154 | 0.0135 | 0.0148 | 0.0092 | 0.0105 | 0.0080 |
| SpaOTsc | CA1 | 0.2704 | 0.2630 | 0.2778 | 0.0556 | 0.0593 | 0.0630 | 0.0444 | 0.0519 | 0.0481 |
| SpaOTsc | CA2/CA3 | 0.3448 | 0.3333 | 0.3678 | 0.0920 | 0.0920 | 0.1149 | 0.0115 | 0.0230 | 0.0805 |
| SpaOTsc | Cajal-Retzius | 0.0866 | 0.0817 | 0.0941 | 0.0262 | 0.0242 | 0.0255 | 0.0153 | 0.0146 | 0.0155 |
| SpaOTsc | Choroid | 0.0960 | 0.1069 | 0.0942 | 0.0507 | 0.0580 | 0.0507 | 0.0562 | 0.0562 | 0.0543 |
| SpaOTsc | Dentate | 0.1411 | 0.1397 | 0.1482 | 0.0319 | 0.0348 | 0.0369 | 0.0195 | 0.0195 | 0.0191 |
| SpaOTsc | Endothelia | 0.1053 | 0.1118 | 0.1020 | 0.0082 | 0.0082 | 0.0115 | 0.0082 | 0.0049 | 0.0049 |
| SpaOTsc | Subiculum Entorhinal (Nxph3+) | 0.2113 | 0.2049 | 0.2254 | 0.0397 | 0.0333 | 0.0423 | 0.0269 | 0.0307 | 0.0269 |
| SpaOTsc | Ependymal | 0.1626 | 0.1602 | 0.1639 | 0.0359 | 0.0383 | 0.0365 | 0.0216 | 0.0198 | 0.0204 |
| SpaOTsc | Fibroblast | 0.1719 | 0.1830 | 0.1853 | 0.0290 | 0.0313 | 0.0402 | 0.0112 | 0.0179 | 0.0223 |
| SpaOTsc | Interneuron (Gad2+) | 0.2603 | 0.2473 | 0.2690 | 0.0564 | 0.0542 | 0.0651 | 0.0282 | 0.0347 | 0.0304 |
| SpaOTsc | Microglia | 0.1095 | 0.1073 | 0.0959 | 0.0220 | 0.0192 | 0.0192 | 0.0085 | 0.0092 | 0.0078 |
| SpaOTsc | Mural | 0.2000 | 0.2000 | 0.2000 | 0.0875 | 0.0500 | 0.0375 | 0.0125 | 0.0250 | 0.0125 |
| SpaOTsc | Neurogenesis | 0.1124 | 0.1032 | 0.1078 | 0.0256 | 0.0235 | 0.0267 | 0.0165 | 0.0158 | 0.0165 |
| SpaOTsc | Oligodendrocyte | 0.1347 | 0.1347 | 0.1399 | 0.0311 | 0.0207 | 0.0259 | 0.0259 | 0.0207 | 0.0155 |
| SpaOTsc | Polydendrocyte | 0.1253 | 0.1192 | 0.1394 | 0.0324 | 0.0272 | 0.0368 | 0.0263 | 0.0245 | 0.0298 |
| SpaOTsc | Subiculum (Slc17a6+) | 0.1600 | 0.1565 | 0.1662 | 0.0381 | 0.0367 | 0.0367 | 0.0201 | 0.0180 | 0.0208 |
| SpaGE | Astrocyte | 0.0074 | 0.0062 | 0.0031 | 0.0037 | 0.0025 | 0.0037 | 0.0012 | 0.0018 | 0.0025 |
| SpaGE | CA1 | 0.0074 | 0.0111 | 0.0037 | 0.0000 | 0.0000 | 0.0000 | 0.0000 | 0.0000 | 0.0000 |
| SpaGE | CA2/CA3 | 0.0115 | 0.0230 | 0.0115 | 0.0115 | 0.0115 | 0.0115 | 0.0000 | 0.0000 | 0.0000 |
| SpaGE | Cajal-Retzius | 0.0092 | 0.0089 | 0.0074 | 0.0052 | 0.0044 | 0.0044 | 0.0070 | 0.0067 | 0.0064 |
| SpaGE | Choroid | 0.0217 | 0.0199 | 0.0236 | 0.0471 | 0.0543 | 0.0598 | 0.0906 | 0.0888 | 0.0888 |
| SpaGE | Dentate | 0.0096 | 0.0099 | 0.0089 | 0.0074 | 0.0053 | 0.0071 | 0.0050 | 0.0060 | 0.0050 |
| SpaGE | Endothelia | 0.0016 | 0.0115 | 0.0099 | 0.0000 | 0.0016 | 0.0016 | 0.0000 | 0.0000 | 0.0000 |
| SpaGE | Subiculum Entorhinal (Nxph3+) | 0.0038 | 0.0038 | 0.0026 | 0.0013 | 0.0013 | 0.0051 | 0.0051 | 0.0064 | 0.0064 |
| SpaGE | Ependymal | 0.0037 | 0.0037 | 0.0080 | 0.0068 | 0.0056 | 0.0087 | 0.0155 | 0.0142 | 0.0130 |
| SpaGE | Fibroblast | 0.0089 | 0.0067 | 0.0089 | 0.0022 | 0.0022 | 0.0045 | 0.0156 | 0.0156 | 0.0156 |
| SpaGE | Interneuron (Gad2+) | 0.0108 | 0.0130 | 0.0087 | 0.0022 | 0.0000 | 0.0000 | 0.0108 | 0.0130 | 0.0108 |
| SpaGE | Microglia | 0.0085 | 0.0085 | 0.0163 | 0.0021 | 0.0021 | 0.0021 | 0.0021 | 0.0014 | 0.0014 |
| SpaGE | Mural | 0.0125 | 0.0500 | 0.0375 | 0.0000 | 0.0000 | 0.0000 | 0.0000 | 0.0000 | 0.0000 |
| SpaGE | Neurogenesis | 0.0119 | 0.0095 | 0.0116 | 0.0032 | 0.0028 | 0.0039 | 0.0035 | 0.0063 | 0.0018 |
| SpaGE | Oligodendrocyte | 0.0155 | 0.0104 | 0.0104 | 0.0000 | 0.0000 | 0.0000 | 0.0000 | 0.0000 | 0.0000 |
| SpaGE | Polydendrocyte | 0.0175 | 0.0175 | 0.0158 | 0.0070 | 0.0096 | 0.0131 | 0.0096 | 0.0105 | 0.0079 |
| SpaGE | Subiculum (Slc17a6+) | 0.0076 | 0.0125 | 0.0069 | 0.0048 | 0.0042 | 0.0062 | 0.0076 | 0.0076 | 0.0069 |
| Seurat | Astrocyte | 0.0031 | 0.0055 | 0.0037 | 0.0006 | 0.0006 | 0.0006 | 0.0037 | 0.0043 | 0.0055 |
| Seurat | CA1 | 0.0148 | 0.0148 | 0.0222 | 0.0111 | 0.0037 | 0.0074 | 0.0111 | 0.0185 | 0.0148 |
| Seurat | CA2/CA3 | 0.0000 | 0.0000 | 0.0000 | 0.0115 | 0.0000 | 0.0000 | 0.0000 | 0.0000 | 0.0000 |
| Seurat | Cajal-Retzius | 0.0030 | 0.0017 | 0.0024 | 0.0020 | 0.0014 | 0.0013 | 0.0019 | 0.0020 | 0.0009 |
| Seurat | Choroid | 0.0036 | 0.0054 | 0.0018 | 0.0018 | 0.0072 | 0.0018 | 0.0236 | 0.0290 | 0.0272 |
| Seurat | Dentate | 0.0039 | 0.0021 | 0.0035 | 0.0032 | 0.0053 | 0.0018 | 0.0053 | 0.0046 | 0.0057 |
| Seurat | Endothelia | 0.0016 | 0.0082 | 0.0049 | 0.0099 | 0.0033 | 0.0016 | 0.0016 | 0.0132 | 0.0099 |
| Seurat | Subiculum Entorhinal (Nxph3+) | 0.0026 | 0.0026 | 0.0038 | 0.0013 | 0.0026 | 0.0051 | 0.0026 | 0.0026 | 0.0051 |
| Seurat | Ependymal | 0.0062 | 0.0043 | 0.0056 | 0.0037 | 0.0043 | 0.0037 | 0.0062 | 0.0068 | 0.0068 |
| Seurat | Fibroblast | 0.0067 | 0.0089 | 0.0045 | 0.0179 | 0.0134 | 0.0067 | 0.0223 | 0.0179 | 0.0089 |
| Seurat | Interneuron (Gad2+) | 0.0087 | 0.0130 | 0.0087 | 0.0108 | 0.0022 | 0.0065 | 0.0087 | 0.0087 | 0.0065 |
| Seurat | Microglia | 0.0071 | 0.0078 | 0.0057 | 0.0028 | 0.0050 | 0.0043 | 0.0036 | 0.0036 | 0.0028 |
| Seurat | Mural | 0.0125 | 0.0125 | 0.0125 | 0.0125 | 0.0375 | 0.0125 | 0.0125 | 0.0250 | 0.0375 |
| Seurat | Neurogenesis | 0.0084 | 0.0056 | 0.0049 | 0.0018 | 0.0021 | 0.0021 | 0.0014 | 0.0011 | 0.0011 |
| Seurat | Oligodendrocyte | 0.0000 | 0.0000 | 0.0000 | 0.0000 | 0.0000 | 0.0000 | 0.0000 | 0.0000 | 0.0000 |
| Seurat | Polydendrocyte | 0.0184 | 0.0131 | 0.0123 | 0.0131 | 0.0114 | 0.0096 | 0.0079 | 0.0070 | 0.0044 |
| Seurat | Subiculum (Slc17a6+) | 0.0028 | 0.0028 | 0.0014 | 0.0042 | 0.0048 | 0.0048 | 0.0104 | 0.0090 | 0.0118 |
| Harmony | Astrocyte | 0.0111 | 0.0037 | 0.0000 | 0.0074 | 0.0025 | 0.0006 | 0.0037 | 0.0006 | 0.0000 |
| Harmony | CA1 | 0.0444 | 0.0259 | 0.0074 | 0.0148 | 0.0074 | 0.0074 | 0.0074 | 0.0037 | 0.0037 |
| Harmony | CA2/CA3 | 0.0460 | 0.0230 | 0.0115 | 0.0115 | 0.0000 | 0.0000 | 0.0000 | 0.0000 | 0.0000 |
| Harmony | Cajal-Retzius | 0.0165 | 0.0083 | 0.0013 | 0.0074 | 0.0037 | 0.0014 | 0.0052 | 0.0026 | 0.0013 |
| Harmony | Choroid | 0.0308 | 0.0308 | 0.0091 | 0.0272 | 0.0254 | 0.0199 | 0.0326 | 0.0308 | 0.0236 |
| Harmony | Dentate | 0.0202 | 0.0099 | 0.0004 | 0.0064 | 0.0043 | 0.0018 | 0.0057 | 0.0032 | 0.0000 |
| Harmony | Endothelia | 0.0181 | 0.0066 | 0.0000 | 0.0033 | 0.0033 | 0.0016 | 0.0016 | 0.0000 | 0.0000 |
| Harmony | Subiculum Entorhinal (Nxph3+) | 0.0256 | 0.0205 | 0.0038 | 0.0102 | 0.0064 | 0.0026 | 0.0051 | 0.0026 | 0.0000 |
| Harmony | Ependymal | 0.0359 | 0.0142 | 0.0068 | 0.0136 | 0.0074 | 0.0025 | 0.0124 | 0.0074 | 0.0043 |
| Harmony | Fibroblast | 0.0179 | 0.0134 | 0.0045 | 0.0045 | 0.0045 | 0.0022 | 0.0022 | 0.0022 | 0.0022 |
| Harmony | Interneuron (Gad2+) | 0.0477 | 0.0325 | 0.0174 | 0.0108 | 0.0065 | 0.0043 | 0.0087 | 0.0043 | 0.0043 |
| Harmony | Microglia | 0.0163 | 0.0107 | 0.0021 | 0.0078 | 0.0036 | 0.0014 | 0.0028 | 0.0014 | 0.0000 |
| Harmony | Mural | 0.0500 | 0.0250 | 0.0250 | 0.0125 | 0.0125 | 0.0125 | 0.0125 | 0.0125 | 0.0000 |
| Harmony | Neurogenesis | 0.0246 | 0.0123 | 0.0035 | 0.0095 | 0.0084 | 0.0028 | 0.0042 | 0.0035 | 0.0011 |
| Harmony | Oligodendrocyte | 0.0104 | 0.0104 | 0.0000 | 0.0104 | 0.0104 | 0.0052 | 0.0000 | 0.0000 | 0.0000 |
| Harmony | Polydendrocyte | 0.0403 | 0.0280 | 0.0131 | 0.0114 | 0.0088 | 0.0070 | 0.0096 | 0.0088 | 0.0096 |
| Harmony | Subiculum (Slc17a6+) | 0.0270 | 0.0180 | 0.0007 | 0.0090 | 0.0083 | 0.0014 | 0.0055 | 0.0035 | 0.0021 |
| LIGER | Astrocyte | 0.0031 | 0.0012 | 0.0031 | 0.0055 | 0.0018 | 0.0031 | 0.0049 | 0.0074 | 0.0018 |
| LIGER | CA1 | 0.0111 | 0.0148 | 0.0111 | 0.0111 | 0.0037 | 0.0000 | 0.0074 | 0.0037 | 0.0037 |
| LIGER | CA2/CA3 | 0.0000 | 0.0000 | 0.0000 | 0.0000 | 0.0115 | 0.0000 | 0.0000 | 0.0000 | 0.0115 |
| LIGER | Cajal-Retzius | 0.0040 | 0.0041 | 0.0031 | 0.0041 | 0.0047 | 0.0026 | 0.0040 | 0.0047 | 0.0031 |
| LIGER | Choroid | 0.0127 | 0.0036 | 0.0072 | 0.0362 | 0.0380 | 0.0145 | 0.0018 | 0.0127 | 0.0018 |
| LIGER | Dentate | 0.0064 | 0.0032 | 0.0018 | 0.0064 | 0.0018 | 0.0035 | 0.0092 | 0.0050 | 0.0014 |
| LIGER | Endothelia | 0.0016 | 0.0049 | 0.0099 | 0.0049 | 0.0000 | 0.0049 | 0.0033 | 0.0082 | 0.0049 |
| LIGER | Subiculum Entorhinal (Nxph3+) | 0.0026 | 0.0051 | 0.0000 | 0.0013 | 0.0026 | 0.0026 | 0.0026 | 0.0013 | 0.0013 |
| LIGER | Ependymal | 0.0031 | 0.0049 | 0.0000 | 0.0056 | 0.0043 | 0.0037 | 0.0012 | 0.0049 | 0.0019 |
| LIGER | Fibroblast | 0.0112 | 0.0179 | 0.0022 | 0.0112 | 0.0112 | 0.0134 | 0.0112 | 0.0067 | 0.0045 |
| LIGER | Interneuron (Gad2+) | 0.0022 | 0.0022 | 0.0000 | 0.0043 | 0.0043 | 0.0000 | 0.0000 | 0.0022 | 0.0000 |
| LIGER | Microglia | 0.0071 | 0.0021 | 0.0036 | 0.0021 | 0.0064 | 0.0028 | 0.0043 | 0.0043 | 0.0021 |
| LIGER | Mural | 0.0125 | 0.0250 | 0.0000 | 0.0375 | 0.0125 | 0.0125 | 0.0000 | 0.0125 | 0.0125 |
| LIGER | Neurogenesis | 0.0046 | 0.0025 | 0.0018 | 0.0032 | 0.0018 | 0.0018 | 0.0032 | 0.0025 | 0.0039 |
| LIGER | Oligodendrocyte | 0.0000 | 0.0052 | 0.0000 | 0.0052 | 0.0052 | 0.0000 | 0.0104 | 0.0000 | 0.0000 |
| LIGER | Polydendrocyte | 0.0096 | 0.0009 | 0.0061 | 0.0061 | 0.0114 | 0.0061 | 0.0070 | 0.0096 | 0.0131 |
| LIGER | Subiculum (Slc17a6+) | 0.0042 | 0.0028 | 0.0021 | 0.0042 | 0.0021 | 0.0028 | 0.0083 | 0.0062 | 0.0048 |
| Pearson correlation | Astrocyte | 0.4754 | 0.4785 | 0.4668 | 0.4133 | 0.4102 | 0.3432 | 0.3149 | 0.2829 | 0.1931 |
| Pearson correlation | CA1 | 0.7444 | 0.7444 | 0.7407 | 0.6741 | 0.6407 | 0.6222 | 0.5333 | 0.5185 | 0.4815 |
| Pearson correlation | CA2/CA3 | 0.9425 | 0.9655 | 0.9195 | 0.8851 | 0.8276 | 0.7241 | 0.5977 | 0.6092 | 0.4598 |
| Pearson correlation | Cajal-Retzius | 0.2285 | 0.2288 | 0.2236 | 0.1710 | 0.1686 | 0.1532 | 0.0992 | 0.0877 | 0.0724 |
| Pearson correlation | Choroid | 0.3025 | 0.3025 | 0.3043 | 0.2047 | 0.2047 | 0.2120 | 0.1630 | 0.1685 | 0.1649 |
| Pearson correlation | Dentate | 0.4475 | 0.4440 | 0.4418 | 0.3741 | 0.3706 | 0.3638 | 0.2443 | 0.2266 | 0.1950 |
| Pearson correlation | Endothelia | 0.7878 | 0.7747 | 0.7697 | 0.7615 | 0.7385 | 0.6694 | 0.7122 | 0.6711 | 0.5033 |
| Pearson correlation | Subiculum Entorhinal (Nxph3+) | 0.6210 | 0.6184 | 0.6184 | 0.5723 | 0.5608 | 0.4994 | 0.4149 | 0.3483 | 0.3060 |
| Pearson correlation | Ependymal | 0.5801 | 0.5745 | 0.5646 | 0.5467 | 0.5393 | 0.5083 | 0.3667 | 0.3531 | 0.3166 |
| Pearson correlation | Fibroblast | 0.7076 | 0.7076 | 0.6853 | 0.6897 | 0.6295 | 0.5402 | 0.4911 | 0.4643 | 0.3728 |
| Pearson correlation | Interneuron (Gad2+) | 0.7354 | 0.7310 | 0.7223 | 0.6963 | 0.6703 | 0.6074 | 0.5315 | 0.5163 | 0.4360 |
| Pearson correlation | Microglia | 0.5593 | 0.5572 | 0.5466 | 0.5110 | 0.4869 | 0.4328 | 0.4407 | 0.3788 | 0.2594 |
| Pearson correlation | Mural | 0.9375 | 0.9250 | 0.9250 | 0.8875 | 0.8625 | 0.7500 | 0.7750 | 0.6875 | 0.5375 |
| Pearson correlation | Neurogenesis | 0.4126 | 0.4108 | 0.4115 | 0.3697 | 0.3592 | 0.3118 | 0.2956 | 0.2616 | 0.1847 |
| Pearson correlation | Oligodendrocyte | 0.8446 | 0.8446 | 0.8342 | 0.6632 | 0.6373 | 0.5078 | 0.4870 | 0.3990 | 0.2435 |
| Pearson correlation | Polydendrocyte | 0.4286 | 0.4224 | 0.4207 | 0.3558 | 0.3418 | 0.3295 | 0.3129 | 0.2989 | 0.2174 |
| Pearson correlation | Subiculum (Slc17a6+) | 0.5270 | 0.5277 | 0.5215 | 0.4785 | 0.4654 | 0.4321 | 0.3130 | 0.3082 | 0.2382 |
| Spearman correlation | Astrocyte | 0.4760 | 0.4742 | 0.4692 | 0.4102 | 0.3979 | 0.3321 | 0.2891 | 0.2460 | 0.1642 |
| Spearman correlation | CA1 | 0.7259 | 0.7222 | 0.7333 | 0.6185 | 0.6037 | 0.5630 | 0.5074 | 0.4815 | 0.4074 |
| Spearman correlation | CA2/CA3 | 0.9195 | 0.9310 | 0.9080 | 0.7701 | 0.7701 | 0.6437 | 0.4713 | 0.4713 | 0.3908 |
| Spearman correlation | Cajal-Retzius | 0.2198 | 0.2191 | 0.2129 | 0.1494 | 0.1468 | 0.1313 | 0.0758 | 0.0674 | 0.0548 |
| Spearman correlation | Choroid | 0.2645 | 0.2572 | 0.2681 | 0.1703 | 0.1757 | 0.1685 | 0.1467 | 0.1449 | 0.1449 |
| Spearman correlation | Dentate | 0.4230 | 0.4223 | 0.4156 | 0.3465 | 0.3351 | 0.3206 | 0.2277 | 0.2128 | 0.1670 |
| Spearman correlation | Endothelia | 0.7780 | 0.7681 | 0.7582 | 0.7566 | 0.7385 | 0.6579 | 0.6579 | 0.6151 | 0.4638 |
| Spearman correlation | Subiculum Entorhinal (Nxph3+) | 0.6031 | 0.5992 | 0.5980 | 0.5262 | 0.5058 | 0.4686 | 0.3483 | 0.3201 | 0.2638 |
| Spearman correlation | Ependymal | 0.5708 | 0.5683 | 0.5597 | 0.5399 | 0.5362 | 0.4954 | 0.4304 | 0.3915 | 0.3389 |
| Spearman correlation | Fibroblast | 0.7076 | 0.7009 | 0.6830 | 0.6786 | 0.6451 | 0.5379 | 0.5335 | 0.4888 | 0.3728 |
| Spearman correlation | Interneuron (Gad2+) | 0.7202 | 0.7267 | 0.7072 | 0.6529 | 0.6464 | 0.5705 | 0.4837 | 0.4794 | 0.3796 |
| Spearman correlation | Microglia | 0.5586 | 0.5608 | 0.5444 | 0.5089 | 0.4712 | 0.4200 | 0.4087 | 0.3475 | 0.2203 |
| Spearman correlation | Mural | 0.9250 | 0.9250 | 0.9125 | 0.8500 | 0.8375 | 0.7000 | 0.7625 | 0.6875 | 0.4375 |
| Spearman correlation | Neurogenesis | 0.3999 | 0.3999 | 0.3996 | 0.3346 | 0.3220 | 0.2777 | 0.2258 | 0.1991 | 0.1478 |
| Spearman correlation | Oligodendrocyte | 0.8187 | 0.8187 | 0.8135 | 0.6736 | 0.6425 | 0.4560 | 0.4715 | 0.3782 | 0.1658 |
| Spearman correlation | Polydendrocyte | 0.4189 | 0.4137 | 0.4093 | 0.3322 | 0.3208 | 0.3006 | 0.3103 | 0.2813 | 0.1963 |
| Spearman correlation | Subiculum (Slc17a6+) | 0.5125 | 0.5152 | 0.5076 | 0.4377 | 0.4404 | 0.3899 | 0.2777 | 0.2618 | 0.1981 |
| Euclidean distance | Astrocyte | 0.0787 | 0.0763 | 0.0910 | 0.0123 | 0.0123 | 0.0123 | 0.0074 | 0.0074 | 0.0074 |
| Euclidean distance | CA1 | 0.4185 | 0.4259 | 0.5370 | 0.1667 | 0.1963 | 0.3259 | 0.0778 | 0.1000 | 0.1667 |
| Euclidean distance | CA2/CA3 | 0.3218 | 0.3218 | 0.4023 | 0.1609 | 0.1724 | 0.2529 | 0.1034 | 0.1034 | 0.1379 |
| Euclidean distance | Cajal-Retzius | 0.0658 | 0.0657 | 0.0748 | 0.0186 | 0.0183 | 0.0215 | 0.0094 | 0.0093 | 0.0106 |
| Euclidean distance | Choroid | 0.1504 | 0.1594 | 0.2156 | 0.0616 | 0.0743 | 0.1196 | 0.0489 | 0.0562 | 0.0978 |
| Euclidean distance | Dentate | 0.1936 | 0.2025 | 0.2642 | 0.0333 | 0.0390 | 0.0883 | 0.0135 | 0.0174 | 0.0266 |
| Euclidean distance | Endothelia | 0.1053 | 0.1003 | 0.1184 | 0.0082 | 0.0082 | 0.0132 | 0.0016 | 0.0016 | 0.0016 |
| Euclidean distance | Subiculum Entorhinal (Nxph3+) | 0.2522 | 0.2574 | 0.3316 | 0.0461 | 0.0576 | 0.1127 | 0.0243 | 0.0282 | 0.0346 |
| Euclidean distance | Ependymal | 0.1880 | 0.1911 | 0.2301 | 0.0377 | 0.0390 | 0.0495 | 0.0186 | 0.0198 | 0.0241 |
| Euclidean distance | Fibroblast | 0.1071 | 0.0982 | 0.1094 | 0.0089 | 0.0067 | 0.0112 | 0.0089 | 0.0067 | 0.0089 |
| Euclidean distance | Interneuron (Gad2+) | 0.2820 | 0.2863 | 0.3861 | 0.0911 | 0.1171 | 0.1779 | 0.0347 | 0.0477 | 0.0434 |
| Euclidean distance | Microglia | 0.0640 | 0.0618 | 0.0640 | 0.0163 | 0.0156 | 0.0163 | 0.0043 | 0.0043 | 0.0043 |
| Euclidean distance | Mural | 0.1000 | 0.1000 | 0.1000 | 0.0250 | 0.0250 | 0.0250 | 0.0125 | 0.0125 | 0.0125 |
| Euclidean distance | Neurogenesis | 0.0871 | 0.0860 | 0.1071 | 0.0197 | 0.0197 | 0.0256 | 0.0109 | 0.0105 | 0.0123 |
| Euclidean distance | Oligodendrocyte | 0.0933 | 0.0933 | 0.0933 | 0.0155 | 0.0155 | 0.0155 | 0.0000 | 0.0000 | 0.0000 |
| Euclidean distance | Polydendrocyte | 0.1306 | 0.1306 | 0.1665 | 0.0184 | 0.0193 | 0.0280 | 0.0096 | 0.0096 | 0.0140 |
| Euclidean distance | Subiculum (Slc17a6+) | 0.1828 | 0.1821 | 0.2486 | 0.0388 | 0.0429 | 0.0796 | 0.0173 | 0.0215 | 0.0270 |

c, Global cell assignment precision, mouse cerebellum

| | | | | | | | | | | |
|---|---|---|---|---|---|---|---|---|---|---|
| Single cell assignment | CytoSPACE | 0.8183 | 0.8077 | 0.7857 | 0.7009 | 0.6815 | 0.5837 | 0.5613 | 0.5104 | 0.3747 |
| precision | | | | | | | | | | |
| Single cell assignment | Tangram (all genes) | 0.7131 | 0.6171 | 0.4449 | 0.5913 | 0.4852 | 0.3356 | 0.4583 | 0.3683 | 0.2346 |
| precision | | | | | | | | | | |
| Single cell assignment | Tangram (top genes) | 0.4719 | 0.4067 | 0.2969 | 0.3215 | 0.2566 | 0.1795 | 0.2349 | 0.1855 | 0.1248 |
| precision | | | | | | | | | | |
| Single cell assignment | CellTrek | 0.0082 | 0.0086 | 0.0064 | 0.0064 | 0.0063 | 0.0047 | 0.0076 | 0.0077 | 0.0063 |
| precision | | | | | | | | | | |
| Single cell assignment | DistMap | 0.1885 | 0.1831 | 0.1674 | 0.0258 | 0.0246 | 0.0206 | 0.0084 | 0.0079 | 0.0078 |
| precision | | | | | | | | | | |
| Single cell assignment | SpaOTsc | 0.1764 | 0.1622 | 0.1495 | 0.0348 | 0.0323 | 0.0299 | 0.0163 | 0.0158 | 0.0157 |
| precision | | | | | | | | | | |
| Single cell assignment | DEEPsc | 0.0007 | 0.0006 | 0.0005 | 0.0005 | 0.0006 | 0.0008 | 0.0010 | 0.0009 | 0.0009 |
| precision | | | | | | | | | | |
| Single cell assignment | SpaGE | 0.0101 | 0.0098 | 0.0090 | 0.0071 | 0.0064 | 0.0056 | 0.0079 | 0.0071 | 0.0083 |
| precision | | | | | | | | | | |
| Single cell assignment | Seurat | 0.0045 | 0.0050 | 0.0043 | 0.0027 | 0.0039 | 0.0027 | 0.0027 | 0.0041 | 0.0039 |
| precision | | | | | | | | | | |
| Single cell assignment | Harmony | 0.0209 | 0.0118 | 0.0017 | 0.0037 | 0.0024 | 0.0008 | 0.0027 | 0.0016 | 0.0004 |
| precision | | | | | | | | | | |
| Single cell assignment | LIGER | 0.0049 | 0.0044 | 0.0027 | 0.0043 | 0.0047 | 0.0032 | 0.0062 | 0.0056 | 0.0045 |
| precision | | | | | | | | | | |
| Single cell assignment | Pearson correlation | 0.5219 | 0.5192 | 0.5125 | 0.4965 | 0.4822 | 0.4352 | 0.4076 | 0.3710 | 0.2834 |
| precision | | | | | | | | | | |
| Single cell assignment | Spearman correlation | 0.5124 | 0.5105 | 0.5023 | 0.4814 | 0.4672 | 0.4150 | 0.3726 | 0.3365 | 0.2499 |
| precision | | | | | | | | | | |
| Single cell assignment | Euclidean distance | 0.1050 | 0.1025 | 0.1231 | 0.0078 | 0.0077 | 0.0121 | 0.0032 | 0.0032 | 0.0039 |
| precision | | | | | | | | | | |

d, Global cell assignment precision, mouse hippocampus

| | | | | | | | | | | |
|---|---|---|---|---|---|---|---|---|---|---|
| Single cell assignment | CytoSPACE | 0.7785 | 0.7667 | 0.7398 | 0.6383 | 0.6068 | 0.5121 | 0.4647 | 0.4143 | 0.3119 |
| precision | | | | | | | | | | |
| Single cell assignment | Tangram (all genes) | 0.6328 | 0.5484 | 0.4163 | 0.4813 | 0.4000 | 0.2803 | 0.3475 | 0.2848 | 0.2024 |
| precision | | | | | | | | | | |
| Single cell assignment | Tangram (top genes) | 0.4106 | 0.3459 | 0.2468 | 0.2822 | 0.2186 | 0.1474 | 0.1823 | 0.1423 | 0.0921 |
| precision | | | | | | | | | | |
| Single cell assignment | CellTrek | 0.0105 | 0.0087 | 0.0079 | 0.0077 | 0.0078 | 0.0065 | 0.0077 | 0.0073 | 0.0075 |
| precision | | | | | | | | | | |
| Single cell assignment | DistMap | 0.1535 | 0.1475 | 0.1018 | 0.0254 | 0.0255 | 0.0181 | 0.0146 | 0.0148 | 0.0114 |
| precision | | | | | | | | | | |
| Single cell assignment | SpaOTsc | 0.1259 | 0.1217 | 0.1291 | 0.0296 | 0.0284 | 0.0305 | 0.0181 | 0.0181 | 0.0185 |
| precision | | | | | | | | | | |
| Single cell assignment | DEEPsc | 0.0006 | 0.0004 | 0.0005 | 0.0010 | 0.0008 | 0.0007 | 0.0015 | 0.0009 | 0.0008 |
| precision | | | | | | | | | | |
| Single cell assignment | SpaGE | 0.0093 | 0.0096 | 0.0092 | 0.0056 | 0.0052 | 0.0064 | 0.0082 | 0.0086 | 0.0075 |
| precision | | | | | | | | | | |
| Single cell assignment | Seurat | 0.0053 | 0.0045 | 0.0043 | 0.0037 | 0.0036 | 0.0028 | 0.0047 | 0.0051 | 0.0047 |
| precision | | | | | | | | | | |
| Single cell assignment | Harmony | 0.0225 | 0.0126 | 0.0031 | 0.0089 | 0.0058 | 0.0026 | 0.0062 | 0.0039 | 0.0021 |
| precision | | | | | | | | | | |
| Single cell assignment | LIGER | 0.0050 | 0.0038 | 0.0028 | 0.0055 | 0.0048 | 0.0034 | 0.0048 | 0.0051 | 0.0033 |
| precision | | | | | | | | | | |
| Single cell assignment | Pearson correlation | 0.4317 | 0.4301 | 0.4248 | 0.3759 | 0.3663 | 0.3337 | 0.2748 | 0.2521 | 0.1981 |
| precision | | | | | | | | | | |
| Single cell assignment | Spearman correlation | 0.4200 | 0.4189 | 0.4128 | 0.3527 | 0.3430 | 0.3066 | 0.2506 | 0.2269 | 0.1725 |
| precision | | | | | | | | | | |
| Single cell assignment | Euclidean distance | 0.1231 | 0.1242 | 0.1547 | 0.0276 | 0.0301 | 0.0473 | 0.0136 | 0.0151 | 0.0197 |
| precision | | | | | | | | | | |

e, Single cell assignment precision by cell type, mouse cerebellum (RCTD fraction estimation)

| | | | | | | | | | | |
|---|---|---|---|---|---|---|---|---|---|---|
| CytoSPACE | Astrocyte | 0.9020 | 0.8702 | 0.8237 | 0.8414 | 0.7945 | 0.6636 | 0.7291 | 0.6836 | 0.5083 |
| CytoSPACE | Bergmann | 0.9035 | 0.8803 | 0.8342 | 0.7964 | 0.7468 | 0.6184 | 0.6542 | 0.5853 | 0.4458 |
| CytoSPACE | Choroid | 0.7890 | 0.7615 | 0.7211 | 0.6495 | 0.6349 | 0.5523 | 0.4697 | 0.4349 | 0.3725 |
| CytoSPACE | Endothelial | 0.8859 | 0.8594 | 0.8360 | 0.8114 | 0.7761 | 0.7277 | 0.7324 | 0.6792 | 0.6120 |
| CytoSPACE | Fibroblast | 0.8546 | 0.8238 | 0.7683 | 0.7566 | 0.7151 | 0.6088 | 0.5972 | 0.5504 | 0.4611 |
| CytoSPACE | Granule | 0.8550 | 0.7969 | 0.6822 | 0.8161 | 0.7363 | 0.5612 | 0.7003 | 0.5854 | 0.3409 |
| CytoSPACE | Microglia | 0.8583 | 0.8587 | 0.7844 | 0.7505 | 0.7177 | 0.5874 | 0.5905 | 0.5382 | 0.3837 |
| CytoSPACE | Interneuron (Nnat+) | 0.8442 | 0.8047 | 0.7554 | 0.7554 | 0.6943 | 0.6055 | 0.6706 | 0.5680 | 0.4675 |
| CytoSPACE | Oligodendrocyte | 0.9012 | 0.8723 | 0.8243 | 0.7918 | 0.7445 | 0.6401 | 0.6239 | 0.5498 | 0.4284 |
| CytoSPACE | Purkinje | 0.8242 | 0.7921 | 0.7019 | 0.6374 | 0.5490 | 0.4084 | 0.4313 | 0.3681 | 0.2445 |
| CytoSPACE | Interneuron (Pvalb+) | 0.8891 | 0.8670 | 0.7990 | 0.7936 | 0.7276 | 0.6188 | 0.6737 | 0.6071 | 0.4653 |
| Tangram (all genes) | Astrocyte | 0.8190 | 0.7389 | 0.5702 | 0.7897 | 0.6598 | 0.4343 | 0.6762 | 0.5156 | 0.3300 |
| Tangram (all genes) | Bergmann | 0.7113 | 0.6002 | 0.4659 | 0.6097 | 0.5207 | 0.3734 | 0.5014 | 0.3883 | 0.2745 |
| Tangram (all genes) | Choroid | 0.7101 | 0.5927 | 0.4550 | 0.7284 | 0.6055 | 0.4239 | 0.6459 | 0.5138 | 0.3633 |
| Tangram (all genes) | Endothelial | 0.8949 | 0.8097 | 0.6428 | 0.8805 | 0.7680 | 0.6076 | 0.8431 | 0.7228 | 0.5168 |
| Tangram (all genes) | Fibroblast | 0.8246 | 0.7116 | 0.5550 | 0.8363 | 0.7037 | 0.5039 | 0.7796 | 0.6263 | 0.4233 |
| Tangram (all genes) | Granule | 0.7004 | 0.5832 | 0.3916 | 0.5402 | 0.4135 | 0.2549 | 0.3563 | 0.2459 | 0.1423 |
| Tangram (all genes) | Microglia | 0.7933 | 0.6959 | 0.5365 | 0.7899 | 0.6273 | 0.4618 | 0.6381 | 0.5362 | 0.3667 |
| Tangram (all genes) | Interneuron (Nnat+) | 0.7653 | 0.6805 | 0.4892 | 0.7298 | 0.6154 | 0.4536 | 0.6252 | 0.5148 | 0.3471 |
| Tangram (all genes) | Oligodendrocyte | 0.7128 | 0.6471 | 0.4975 | 0.6972 | 0.5914 | 0.4100 | 0.5928 | 0.4594 | 0.3331 |
| Tangram (all genes) | Purkinje | 0.5169 | 0.4414 | 0.3040 | 0.3773 | 0.3109 | 0.1918 | 0.2491 | 0.2019 | 0.1163 |
| Tangram (all genes) | Interneuron (Pvalb+) | 0.7765 | 0.6542 | 0.5139 | 0.7104 | 0.6044 | 0.4064 | 0.5914 | 0.4725 | 0.2952 |
| Tangram (marker genes) | Astrocyte | 0.5762 | 0.4838 | 0.3815 | 0.4474 | 0.3333 | 0.2034 | 0.2872 | 0.1738 | 0.1155 |
| Tangram (marker genes) | Bergmann | 0.5750 | 0.4529 | 0.3458 | 0.4242 | 0.3182 | 0.2143 | 0.3017 | 0.1957 | 0.1205 |
| Tangram (marker genes) | Choroid | 0.7706 | 0.6771 | 0.4917 | 0.7028 | 0.5450 | 0.3174 | 0.5982 | 0.4037 | 0.2202 |
| Tangram (marker genes) | Endothelial | 0.8303 | 0.7526 | 0.6193 | 0.7559 | 0.7141 | 0.5271 | 0.6660 | 0.5248 | 0.4100 |
| Tangram (marker genes) | Fibroblast | 0.7991 | 0.7273 | 0.5998 | 0.7655 | 0.6810 | 0.5017 | 0.6623 | 0.5528 | 0.3902 |
| Tangram (marker genes) | Granule | 0.2696 | 0.2017 | 0.1106 | 0.1151 | 0.0881 | 0.0447 | 0.0551 | 0.0429 | 0.0260 |
| Tangram (marker genes) | Microglia | 0.8760 | 0.7621 | 0.5823 | 0.7987 | 0.6624 | 0.4839 | 0.6405 | 0.5166 | 0.3243 |
| Tangram (marker genes) | Interneuron (Nnat+) | 0.5937 | 0.4517 | 0.2821 | 0.4339 | 0.2663 | 0.1736 | 0.3097 | 0.1637 | 0.0789 |
| Tangram (marker genes) | Oligodendrocyte | 0.6366 | 0.5681 | 0.4093 | 0.5208 | 0.4255 | 0.2646 | 0.3769 | 0.2668 | 0.1764 |
| Tangram (marker genes) | Purkinje | 0.4634 | 0.3878 | 0.2601 | 0.3100 | 0.2198 | 0.1223 | 0.1708 | 0.1190 | 0.0673 |
| Tangram (marker genes) | Interneuron (Pvalb+) | 0.5408 | 0.4062 | 0.2967 | 0.3964 | 0.2618 | 0.1479 | 0.2896 | 0.1626 | 0.0762 |
| CellTrek | Astrocyte | 0.0218 | 0.0261 | 0.0071 | 0.0063 | 0.0095 | 0.0128 | 0.0243 | 0.0090 | 0.0094 |
| CellTrek | Bergmann | 0.0149 | 0.0190 | 0.0180 | 0.0157 | 0.0115 | 0.0080 | 0.0132 | 0.0110 | 0.0124 |
| CellTrek | Choroid | 0.1068 | 0.1132 | 0.0850 | 0.0882 | 0.0921 | 0.0830 | 0.0522 | 0.0609 | 0.0772 |
| CellTrek | Endothelial | 0.0410 | 0.0324 | 0.0307 | 0.0262 | 0.0199 | 0.0133 | 0.0151 | 0.0176 | 0.0079 |
| CellTrek | Fibroblast | 0.0907 | 0.0941 | 0.0668 | 0.0709 | 0.0548 | 0.0490 | 0.0412 | 0.0387 | 0.0334 |
| CellTrek | Granule | 0.0092 | 0.0089 | 0.0072 | 0.0064 | 0.0054 | 0.0056 | 0.0084 | 0.0097 | 0.0091 |
| CellTrek | Microglia | 0.0599 | 0.0732 | 0.0548 | 0.0563 | 0.0634 | 0.0465 | 0.0599 | 0.0482 | 0.0473 |
| CellTrek | Interneuron (Nnat+) | 0.0575 | 0.0461 | 0.0319 | 0.0330 | 0.0397 | 0.0376 | 0.0263 | 0.0348 | 0.0308 |
| CellTrek | Oligodendrocyte | 0.0202 | 0.0197 | 0.0220 | 0.0187 | 0.0201 | 0.0100 | 0.0384 | 0.0318 | 0.0276 |
| CellTrek | Purkinje | 0.0380 | 0.0344 | 0.0257 | 0.0239 | 0.0271 | 0.0210 | 0.0347 | 0.0289 | 0.0299 |
| CellTrek | Interneuron (Pvalb+) | 0.0177 | 0.0196 | 0.0151 | 0.0120 | 0.0079 | 0.0145 | 0.0195 | 0.0147 | 0.0109 |
| Euclidean distance | Astrocyte | 0.1007 | 0.0993 | 0.1139 | 0.0066 | 0.0066 | 0.0079 | 0.0013 | 0.0013 | 0.0013 |
| Euclidean distance | Bergmann | 0.1122 | 0.1107 | 0.1426 | 0.0079 | 0.0079 | 0.0142 | 0.0032 | 0.0032 | 0.0043 |
| Euclidean distance | Choroid | 0.1083 | 0.1046 | 0.1303 | 0.0147 | 0.0147 | 0.0330 | 0.0037 | 0.0037 | 0.0128 |
| Euclidean distance | Endothelial | 0.1991 | 0.1962 | 0.2372 | 0.0088 | 0.0088 | 0.0161 | 0.0015 | 0.0015 | 0.0029 |
| Euclidean distance | Fibroblast | 0.1506 | 0.1506 | 0.1867 | 0.0201 | 0.0221 | 0.0311 | 0.0070 | 0.0070 | 0.0090 |
| Euclidean distance | Granule | 0.0886 | 0.0841 | 0.0976 | 0.0070 | 0.0067 | 0.0077 | 0.0031 | 0.0031 | 0.0032 |
| Euclidean distance | Microglia | 0.0917 | 0.0951 | 0.0968 | 0.0051 | 0.0051 | 0.0068 | 0.0034 | 0.0034 | 0.0034 |
| Euclidean distance | Interneuron (Nnat+) | 0.1854 | 0.1933 | 0.2268 | 0.0138 | 0.0138 | 0.0592 | 0.0059 | 0.0059 | 0.0099 |
| Euclidean distance | Oligodendrocyte | 0.1489 | 0.1524 | 0.1976 | 0.0042 | 0.0042 | 0.0141 | 0.0007 | 0.0007 | 0.0007 |
| Euclidean distance | Purkinje | 0.0650 | 0.0636 | 0.0691 | 0.0069 | 0.0064 | 0.0073 | 0.0041 | 0.0041 | 0.0046 |
| Euclidean distance | Interneuron (Pvalb+) | 0.1550 | 0.1525 | 0.1982 | 0.0073 | 0.0073 | 0.0147 | 0.0024 | 0.0024 | 0.0033 |
| Pearson correlation | Astrocyte | 0.6053 | 0.5974 | 0.5881 | 0.6132 | 0.5788 | 0.5470 | 0.5245 | 0.5007 | 0.4079 |
| Pearson correlation | Bergmann | 0.4356 | 0.4313 | 0.4238 | 0.4147 | 0.4053 | 0.3816 | 0.3454 | 0.3301 | 0.2753 |
| Pearson correlation | Choroid | 0.2752 | 0.2697 | 0.2661 | 0.2147 | 0.2110 | 0.1982 | 0.1835 | 0.1798 | 0.1706 |
| Pearson correlation | Endothelial | 0.6340 | 0.6296 | 0.6384 | 0.6047 | 0.5915 | 0.5681 | 0.5520 | 0.5344 | 0.5066 |
| Pearson correlation | Fibroblast | 0.4709 | 0.4699 | 0.4518 | 0.4247 | 0.4187 | 0.3916 | 0.3805 | 0.3624 | 0.3474 |
| Pearson correlation | Granule | 0.6298 | 0.6279 | 0.6179 | 0.5916 | 0.5718 | 0.4998 | 0.4714 | 0.4133 | 0.2807 |
| Pearson correlation | Microglia | 0.2954 | 0.3022 | 0.2920 | 0.3633 | 0.3752 | 0.3413 | 0.3158 | 0.3277 | 0.2581 |
| Pearson correlation | Interneuron (Nnat+) | 0.4615 | 0.4497 | 0.4675 | 0.4320 | 0.4241 | 0.4241 | 0.3807 | 0.3432 | 0.3353 |
| Pearson correlation | Oligodendrocyte | 0.4488 | 0.4446 | 0.4474 | 0.4510 | 0.4375 | 0.4178 | 0.3881 | 0.3585 | 0.3155 |
| Pearson correlation | Purkinje | 0.1685 | 0.1703 | 0.1685 | 0.1754 | 0.1726 | 0.1571 | 0.1415 | 0.1346 | 0.1117 |
| Pearson correlation | Interneuron (Pvalb+) | 0.5351 | 0.5253 | 0.5261 | 0.5082 | 0.5008 | 0.4804 | 0.4372 | 0.4339 | 0.3825 |

f, Single cell assignment precision by cell type, mouse hippocampus (RCTD fraction estimation)

| | | | | | | | | | | |
|---|---|---|---|---|---|---|---|---|---|---|
| CytoSPACE | Astrocyte | 0.8635 | 0.8518 | 0.8069 | 0.7401 | 0.6827 | 0.5517 | 0.5179 | 0.4476 | 0.2897 |
| CytoSPACE | CA1 | 0.7889 | 0.7630 | 0.7519 | 0.7074 | 0.6889 | 0.6556 | 0.6593 | 0.6296 | 0.5630 |
| CytoSPACE | CA2/CA3 | 0.9540 | 0.9080 | 0.8851 | 0.7356 | 0.7241 | 0.5977 | 0.6552 | 0.6322 | 0.4483 |
| CytoSPACE | Cajal-Retzius | 0.7119 | 0.6507 | 0.5326 | 0.5018 | 0.4403 | 0.3051 | 0.3273 | 0.2557 | 0.1580 |
| CytoSPACE | Choroid | 0.7351 | 0.7049 | 0.6589 | 0.5124 | 0.4576 | 0.3764 | 0.3394 | 0.3595 | 0.3284 |
| CytoSPACE | Dentate | 0.7748 | 0.7521 | 0.7025 | 0.6344 | 0.5887 | 0.5220 | 0.4777 | 0.4333 | 0.3195 |
| CytoSPACE | Endothelial | 0.9260 | 0.9228 | 0.9227 | 0.8776 | 0.8445 | 0.7607 | 0.6749 | 0.6563 | 0.5152 |
| CytoSPACE | Subiculum Entorhinal (Nxph3+) | 0.8835 | 0.8361 | 0.7951 | 0.7708 | 0.7465 | 0.6440 | 0.6338 | 0.5723 | 0.4545 |
| CytoSPACE | Ependymal | 0.8740 | 0.8766 | 0.8476 | 0.7128 | 0.7078 | 0.6288 | 0.5285 | 0.4997 | 0.4102 |
| CytoSPACE | Fibroblast | 0.8462 | 0.8612 | 0.8357 | 0.8071 | 0.7473 | 0.6817 | 0.6088 | 0.5820 | 0.4403 |
| CytoSPACE | Interneuron (Gad2+) | 0.8547 | 0.8438 | 0.8026 | 0.7722 | 0.7397 | 0.6681 | 0.6725 | 0.6508 | 0.5315 |
| CytoSPACE | Microglia | 0.8128 | 0.8020 | 0.7820 | 0.7468 | 0.6946 | 0.5930 | 0.5695 | 0.4404 | 0.3083 |
| CytoSPACE | Mural | 0.9500 | 0.9750 | 0.9125 | 0.8500 | 0.8375 | 0.7375 | 0.7375 | 0.6375 | 0.4500 |
| CytoSPACE | Neurogenesis | 0.7684 | 0.7361 | 0.6835 | 0.6046 | 0.5713 | 0.4221 | 0.4522 | 0.3966 | 0.2476 |
| CytoSPACE | Oligodendrocyte | 0.8964 | 0.8860 | 0.8808 | 0.8083 | 0.7358 | 0.5233 | 0.5492 | 0.4715 | 0.2865 |
| CytoSPACE | Polydendrocyte | 0.8624 | 0.8440 | 0.7984 | 0.6582 | 0.6039 | 0.5162 | 0.4382 | 0.4014 | 0.2883 |
| CytoSPACE | Subiculum (Slc17a6+) | 0.8249 | 0.7987 | 0.7208 | 0.7004 | 0.6372 | 0.5480 | 0.5356 | 0.4800 | 0.3607 |
| Tangram (all genes) | Astrocyte | 0.5787 | 0.4625 | 0.3241 | 0.3395 | 0.2620 | 0.1734 | 0.2174 | 0.1565 | 0.0978 |
| Tangram (all genes) | CA1 | 0.7852 | 0.6815 | 0.6259 | 0.7185 | 0.6815 | 0.6185 | 0.6407 | 0.5407 | 0.4593 |
| Tangram (all genes) | CA2/CA3 | 0.6552 | 0.6207 | 0.4483 | 0.5287 | 0.4598 | 0.3793 | 0.4368 | 0.3678 | 0.3103 |
| Tangram (all genes) | Cajal-Retzius | 0.4281 | 0.3512 | 0.2262 | 0.2214 | 0.1693 | 0.1121 | 0.1140 | 0.0875 | 0.0610 |
| Tangram (all genes) | Choroid | 0.5680 | 0.5488 | 0.3618 | 0.4814 | 0.4102 | 0.3118 | 0.4281 | 0.3656 | 0.3373 |
| Tangram (all genes) | Dentate | 0.6418 | 0.5745 | 0.4461 | 0.5039 | 0.4092 | 0.3121 | 0.3691 | 0.2943 | 0.2121 |
| Tangram (all genes) | Endothelial | 0.8580 | 0.7702 | 0.5888 | 0.7522 | 0.6218 | 0.4107 | 0.5556 | 0.4281 | 0.2684 |
| Tangram (all genes) | Subiculum Entorhinal (Nxph3+) | 0.7542 | 0.7004 | 0.5506 | 0.6415 | 0.5198 | 0.3931 | 0.4840 | 0.3803 | 0.3150 |
| Tangram (all genes) | Ependymal | 0.8252 | 0.7307 | 0.5768 | 0.7045 | 0.5924 | 0.4115 | 0.5118 | 0.4202 | 0.2676 |
| Tangram (all genes) | Fibroblast | 0.8333 | 0.6751 | 0.5620 | 0.7536 | 0.5714 | 0.4212 | 0.5680 | 0.4341 | 0.2662 |
| Tangram (all genes) | Interneuron (Gad2+) | 0.8286 | 0.7267 | 0.6161 | 0.7484 | 0.6638 | 0.4664 | 0.6139 | 0.5618 | 0.4555 |
| Tangram (all genes) | Microglia | 0.7733 | 0.6917 | 0.4541 | 0.6388 | 0.4716 | 0.2909 | 0.4611 | 0.3119 | 0.1767 |
| Tangram (all genes) | Mural | 0.9000 | 0.8750 | 0.7125 | 0.9125 | 0.8125 | 0.6500 | 0.8000 | 0.6500 | 0.4250 |
| Tangram (all genes) | Neurogenesis | 0.5718 | 0.4778 | 0.3571 | 0.3913 | 0.3103 | 0.1851 | 0.2451 | 0.1729 | 0.1371 |
| Tangram (all genes) | Oligodendrocyte | 0.6632 | 0.4663 | 0.3316 | 0.3731 | 0.2539 | 0.1813 | 0.2642 | 0.1606 | 0.0938 |
| Tangram (all genes) | Polydendrocyte | 0.6468 | 0.5670 | 0.4356 | 0.5206 | 0.4154 | 0.2962 | 0.3900 | 0.3295 | 0.2279 |
| Tangram (all genes) | Subiculum (Slc17a6+) | 0.6823 | 0.6154 | 0.4579 | 0.5838 | 0.4563 | 0.3306 | 0.4342 | 0.3333 | 0.2497 |
| Tangram (marker genes) | Astrocyte | 0.4434 | 0.3284 | 0.1974 | 0.2624 | 0.1531 | 0.0990 | 0.1568 | 0.0891 | 0.0504 |
| Tangram (marker genes) | CA1 | 0.4926 | 0.4259 | 0.3741 | 0.3630 | 0.3481 | 0.2444 | 0.2481 | 0.2370 | 0.1704 |
| Tangram (marker genes) | CA2/CA3 | 0.3678 | 0.3333 | 0.3103 | 0.2989 | 0.3448 | 0.1954 | 0.2874 | 0.2069 | 0.1609 |
| Tangram (marker genes) | Cajal-Retzius | 0.2176 | 0.1451 | 0.0765 | 0.0887 | 0.0530 | 0.0288 | 0.0423 | 0.0293 | 0.0194 |
| Tangram (marker genes) | Choroid | 0.4726 | 0.4293 | 0.3695 | 0.2888 | 0.2712 | 0.2776 | 0.2477 | 0.2296 | 0.2358 |
| Tangram (marker genes) | Dentate | 0.3096 | 0.2415 | 0.1511 | 0.1773 | 0.1220 | 0.0652 | 0.0929 | 0.0649 | 0.0450 |
| Tangram (marker genes) | Endothelial | 0.8360 | 0.7281 | 0.5592 | 0.7493 | 0.6125 | 0.4214 | 0.5473 | 0.4625 | 0.2922 |
| Tangram (marker genes) | Subiculum Entorhinal (Nxph3+) | 0.3739 | 0.2638 | 0.1857 | 0.2087 | 0.1255 | 0.0973 | 0.1255 | 0.0743 | 0.0666 |
| Tangram (marker genes) | Ependymal | 0.5759 | 0.4541 | 0.2900 | 0.3877 | 0.2842 | 0.1486 | 0.2375 | 0.1638 | 0.0915 |
| Tangram (marker genes) | Fibroblast | 0.8526 | 0.6688 | 0.5418 | 0.6143 | 0.4066 | 0.3055 | 0.4320 | 0.2605 | 0.1741 |
| Tangram (marker genes) | Interneuron (Gad2+) | 0.5792 | 0.5054 | 0.4642 | 0.4447 | 0.4165 | 0.3232 | 0.3145 | 0.2928 | 0.2668 |
| Tangram (marker genes) | Microglia | 0.7581 | 0.6780 | 0.4501 | 0.7031 | 0.5271 | 0.2909 | 0.4763 | 0.3242 | 0.1767 |
| Tangram (marker genes) | Mural | 0.9000 | 0.9000 | 0.8250 | 0.8625 | 0.8625 | 0.7875 | 0.7625 | 0.7625 | 0.6625 |
| Tangram (marker genes) | Neurogenesis | 0.2510 | 0.2239 | 0.1483 | 0.1433 | 0.0980 | 0.0588 | 0.0755 | 0.0472 | 0.0372 |
| Tangram (marker genes) | Oligodendrocyte | 0.4249 | 0.3420 | 0.2228 | 0.2176 | 0.1451 | 0.0674 | 0.1036 | 0.0777 | 0.0469 |
| Tangram (marker genes) | Polydendrocyte | 0.5855 | 0.4838 | 0.3918 | 0.4443 | 0.3453 | 0.2594 | 0.3427 | 0.2805 | 0.1946 |
| Tangram (marker genes) | Subiculum (Slc17a6+) | 0.3151 | 0.2393 | 0.1675 | 0.1813 | 0.1193 | 0.0704 | 0.1005 | 0.0644 | 0.0377 |
| CellTrek | Astrocyte | 0.0067 | 0.0055 | 0.0075 | 0.0094 | 0.0081 | 0.0041 | 0.0061 | 0.0108 | 0.0068 |
| CellTrek | CA1 | 0.0609 | 0.0661 | 0.0852 | 0.0649 | 0.0672 | 0.0611 | 0.0608 | 0.0720 | 0.0749 |
| CellTrek | CA2/CA3 | 0.0704 | 0.0395 | 0.0351 | 0.0133 | 0.0127 | 0.0128 | 0.0000 | 0.0000 | 0.0000 |
| CellTrek | Cajal-Retzius | 0.0190 | 0.0125 | 0.0115 | 0.0148 | 0.0101 | 0.0094 | 0.0126 | 0.0097 | 0.0087 |
| CellTrek | Choroid | 0.0412 | 0.0316 | 0.0333 | 0.0470 | 0.0599 | 0.0460 | 0.0589 | 0.0504 | 0.0657 |
| CellTrek | Dentate | 0.0238 | 0.0228 | 0.0168 | 0.0210 | 0.0175 | 0.0135 | 0.0186 | 0.0137 | 0.0154 |
| CellTrek | Endothelial | 0.0078 | 0.0097 | 0.0157 | 0.0141 | 0.0188 | 0.0144 | 0.0252 | 0.0208 | 0.0090 |
| CellTrek | Subiculum Entorhinal (Nxph3+) | 0.0164 | 0.0194 | 0.0198 | 0.0170 | 0.0216 | 0.0244 | 0.0218 | 0.0125 | 0.0107 |
| CellTrek | Ependymal | 0.0233 | 0.0169 | 0.0136 | 0.0083 | 0.0142 | 0.0091 | 0.0178 | 0.0185 | 0.0145 |
| CellTrek | Fibroblast | 0.0560 | 0.0435 | 0.0208 | 0.0387 | 0.0567 | 0.0516 | 0.0541 | 0.0708 | 0.0477 |
| CellTrek | Interneuron (Gad2+) | 0.0690 | 0.0519 | 0.0784 | 0.0265 | 0.0426 | 0.0463 | 0.0420 | 0.0266 | 0.0373 |
| CellTrek | Microglia | 0.0327 | 0.0352 | 0.0212 | 0.0177 | 0.0195 | 0.0176 | 0.0123 | 0.0151 | 0.0147 |
| CellTrek | Mural | 0.1333 | 0.0727 | 0.1486 | 0.1500 | 0.0759 | 0.1500 | 0.0625 | 0.0256 | 0.0513 |
| CellTrek | Neurogenesis | 0.0335 | 0.0325 | 0.0259 | 0.0128 | 0.0122 | 0.0100 | 0.0098 | 0.0103 | 0.0090 |
| CellTrek | Oligodendrocyte | 0.0154 | 0.0141 | 0.0135 | 0.0134 | 0.0000 | 0.0195 | 0.0398 | 0.0337 | 0.0174 |
| CellTrek | Polydendrocyte | 0.0811 | 0.0735 | 0.0697 | 0.0543 | 0.0547 | 0.0455 | 0.0424 | 0.0541 | 0.0495 |
| CellTrek | Subiculum (Slc17a6+) | 0.0191 | 0.0299 | 0.0281 | 0.0146 | 0.0205 | 0.0200 | 0.0154 | 0.0237 | 0.0185 |
| Euclidean distance | Astrocyte | 0.0787 | 0.0763 | 0.0910 | 0.0123 | 0.0123 | 0.0123 | 0.0074 | 0.0074 | 0.0074 |
| Euclidean distance | CA1 | 0.4185 | 0.4259 | 0.5370 | 0.1667 | 0.1963 | 0.3259 | 0.0778 | 0.1000 | 0.1667 |
| Euclidean distance | CA2/CA3 | 0.3218 | 0.3218 | 0.4023 | 0.1609 | 0.1724 | 0.2529 | 0.1034 | 0.1034 | 0.1379 |
| Euclidean distance | Cajal-Retzius | 0.0658 | 0.0657 | 0.0748 | 0.0186 | 0.0183 | 0.0215 | 0.0094 | 0.0093 | 0.0106 |
| Euclidean distance | Choroid | 0.1504 | 0.1594 | 0.2156 | 0.0616 | 0.0743 | 0.1196 | 0.0489 | 0.0562 | 0.0978 |
| Euclidean distance | Dentate | 0.1936 | 0.2025 | 0.2642 | 0.0333 | 0.0390 | 0.0883 | 0.0135 | 0.0174 | 0.0266 |
| Euclidean distance | Endothelial | 0.1053 | 0.1003 | 0.1184 | 0.0082 | 0.0082 | 0.0132 | 0.0016 | 0.0016 | 0.0016 |
| Euclidean distance | Subiculum Entorhinal (Nxph3+) | 0.2522 | 0.2574 | 0.3316 | 0.0461 | 0.0576 | 0.1127 | 0.0243 | 0.0282 | 0.0346 |
| Euclidean distance | Ependymal | 0.1880 | 0.1911 | 0.2301 | 0.0377 | 0.0390 | 0.0495 | 0.0186 | 0.0198 | 0.0241 |
| Euclidean distance | Fibroblast | 0.1071 | 0.0982 | 0.1094 | 0.0089 | 0.0067 | 0.0112 | 0.0089 | 0.0067 | 0.0089 |
| Euclidean distance | Interneuron (Gad2+) | 0.2820 | 0.2863 | 0.3861 | 0.0911 | 0.1171 | 0.1779 | 0.0347 | 0.0477 | 0.0434 |
| Euclidean distance | Microglia | 0.0640 | 0.0618 | 0.0640 | 0.0163 | 0.0156 | 0.0163 | 0.0043 | 0.0043 | 0.0043 |
| Euclidean distance | Mural | 0.1000 | 0.1000 | 0.1000 | 0.0250 | 0.0250 | 0.0250 | 0.0125 | 0.0125 | 0.0125 |
| Euclidean distance | Neurogenesis | 0.0871 | 0.0860 | 0.1071 | 0.0197 | 0.0197 | 0.0256 | 0.0109 | 0.0105 | 0.0123 |
| Euclidean distance | Oligodendrocyte | 0.0933 | 0.0933 | 0.0933 | 0.0155 | 0.0155 | 0.0155 | 0.0000 | 0.0000 | 0.0000 |
| Euclidean distance | Polydendrocyte | 0.1306 | 0.1306 | 0.1665 | 0.0184 | 0.0193 | 0.0280 | 0.0096 | 0.0096 | 0.0140 |
| Euclidean distance | Subiculum (Slc17a6+) | 0.1828 | 0.1821 | 0.2486 | 0.0388 | 0.0429 | 0.0796 | 0.0173 | 0.0215 | 0.0270 |
| Pearson correlation | Astrocyte | 0.4754 | 0.4785 | 0.4668 | 0.4133 | 0.4102 | 0.3432 | 0.3149 | 0.2829 | 0.1931 |
| Pearson correlation | CA1 | 0.7444 | 0.7444 | 0.7407 | 0.6741 | 0.6407 | 0.6222 | 0.5333 | 0.5185 | 0.4815 |
| Pearson correlation | CA2/CA3 | 0.9425 | 0.9655 | 0.9195 | 0.8851 | 0.8276 | 0.7241 | 0.5977 | 0.6092 | 0.4598 |
| Pearson correlation | Cajal-Retzius | 0.2285 | 0.2288 | 0.2236 | 0.1710 | 0.1686 | 0.1532 | 0.0992 | 0.0877 | 0.0724 |
| Pearson correlation | Choroid | 0.3025 | 0.3025 | 0.3043 | 0.2047 | 0.2047 | 0.2120 | 0.1630 | 0.1685 | 0.1649 |
| Pearson correlation | Dentate | 0.4475 | 0.4440 | 0.4418 | 0.3741 | 0.3706 | 0.3638 | 0.2443 | 0.2266 | 0.1950 |
| Pearson correlation | Endothelial | 0.7878 | 0.7747 | 0.7697 | 0.7615 | 0.7385 | 0.6694 | 0.7122 | 0.6711 | 0.5033 |
| Pearson correlation | Subiculum Entorhinal (Nxph3+) | 0.6210 | 0.6184 | 0.6184 | 0.5723 | 0.5608 | 0.4994 | 0.4149 | 0.3483 | 0.3060 |
| Pearson correlation | Ependymal | 0.5801 | 0.5745 | 0.5646 | 0.5467 | 0.5393 | 0.5083 | 0.3667 | 0.3531 | 0.3166 |
| Pearson correlation | Fibroblast | 0.7076 | 0.7076 | 0.6853 | 0.6897 | 0.6295 | 0.5402 | 0.4911 | 0.4643 | 0.3728 |
| Pearson correlation | Interneuron (Gad2+) | 0.7354 | 0.7310 | 0.7223 | 0.6963 | 0.6703 | 0.6074 | 0.5315 | 0.5163 | 0.4360 |
| Pearson correlation | Microglia | 0.5593 | 0.5572 | 0.5466 | 0.5110 | 0.4869 | 0.4328 | 0.4407 | 0.3788 | 0.2594 |
| Pearson correlation | Mural | 0.9375 | 0.9250 | 0.9250 | 0.8875 | 0.8625 | 0.7500 | 0.7750 | 0.6875 | 0.5375 |
| Pearson correlation | Neurogenesis | 0.4126 | 0.4108 | 0.4115 | 0.3697 | 0.3592 | 0.3118 | 0.2956 | 0.2616 | 0.1847 |
| Pearson correlation | Oligodendrocyte | 0.8446 | 0.8446 | 0.8342 | 0.6632 | 0.6373 | 0.5078 | 0.4870 | 0.3990 | 0.2435 |
| Pearson correlation | Polydendrocyte | 0.4286 | 0.4224 | 0.4207 | 0.3558 | 0.3418 | 0.3295 | 0.3129 | 0.2989 | 0.2174 |
| Pearson correlation | Subiculum (Slc17a6+) | 0.5270 | 0.5277 | 0.5215 | 0.4785 | 0.4654 | 0.4321 | 0.3130 | 0.3082 | 0.2382 |

g, Global cell assignment precision, mouse cerebellum (RCTD fraction estimation)

| | | | | | | | | | | |
|---|---|---|---|---|---|---|---|---|---|---|
| Single cell assignment | CytoSPACE | 0.8643 | 0.8281 | 0.7656 | 0.7798 | 0.7113 | 0.5780 | 0.6437 | 0.5535 | 0.3961 |
| precision | | | | | | | | | | |
| Single cell assignment | Tangram (all genes) | 0.7080 | 0.6070 | 0.4559 | 0.6015 | 0.4994 | 0.3577 | 0.4563 | 0.3607 | 0.2594 |
| precision | | | | | | | | | | |
| Single cell assignment | Tangram (marker genes) | 0.4746 | 0.4112 | 0.3336 | 0.3279 | 0.2785 | 0.2131 | 0.2225 | 0.1745 | 0.1352 |
| precision | | | | | | | | | | |
| Single cell assignment | CellTrek | 0.0273 | 0.0286 | 0.0222 | 0.0188 | 0.0184 | 0.0155 | 0.0196 | 0.0187 | 0.0178 |
| precision | | | | | | | | | | |
| Single cell assignment | Euclidean distance | 0.1050 | 0.1025 | 0.1231 | 0.0078 | 0.0077 | 0.0121 | 0.0032 | 0.0032 | 0.0039 |
| precision | | | | | | | | | | |
| Single cell assignment | Pearson correlation | 0.5219 | 0.5192 | 0.5125 | 0.4965 | 0.4822 | 0.4352 | 0.4076 | 0.3710 | 0.2834 |
| precision | | | | | | | | | | |

h, Global cell assignment precision, mouse hippocampus (RCTD fraction estimation)

| | | | | | | | | | | |
|---|---|---|---|---|---|---|---|---|---|---|
| Single cell assignment | CytoSPACE | 0.7976 | 0.7730 | 0.7266 | 0.6333 | 0.5925 | 0.5055 | 0.4542 | 0.4033 | 0.3026 |
| precision | | | | | | | | | | |
| Single cell assignment | Tangram (all genes) | 0.6180 | 0.5432 | 0.4169 | 0.4431 | 0.3637 | 0.2717 | 0.3038 | 0.2453 | 0.1845 |
| precision | | | | | | | | | | |
| Single cell assignment | Tangram (marker genes) | 0.3942 | 0.3222 | 0.2364 | 0.2400 | 0.1811 | 0.1277 | 0.1450 | 0.1111 | 0.0835 |
| precision | | | | | | | | | | |
| Single cell assignment | CellTrek | 0.0286 | 0.0257 | 0.0237 | 0.0198 | 0.0199 | 0.0179 | 0.0195 | 0.0188 | 0.0175 |
| precision | | | | | | | | | | |
| Single cell assignment | Euclidean distance | 0.1231 | 0.1242 | 0.1547 | 0.0276 | 0.0301 | 0.0473 | 0.0136 | 0.0151 | 0.0197 |
| precision | | | | | | | | | | |
| Single cell assignment | Pearson correlation | 0.4317 | 0.4301 | 0.4248 | 0.3759 | 0.3663 | 0.3337 | 0.2748 | 0.2521 | 0.1981 |
| precision | | | | | | | | | | |

a-b, Fraction of cells per cell type mapped to correct spot in ST data across noise levels and spatial resolutions by method in mouse cerebellum (a) and mouse hippocampus (b) samples.
c-d, Global assignment precision across noise levels and spatial resolutions by method in mouse cerebellum (c) and mouse hippocampus (d) samples. Single cell assignment precision is the fraction of cells correctly mapped to corresponding ground truth spots in ST data, and cell type precision is the fraction of cells mapped to spots n ST data containing at least one cell of the same cell type in ground truth.
e-h, same as a-d respectively but for selected methods with RCTD used for cell type fraction estimation for CytoSPACE input rather than Spatial Seurat.

TABLE 2
scRNA-seq annotations.
Mapping of cell subsets from the Wu et al. (BRCA) and Lee et al. (CRC)
scRNA-seq datasets to corresponding cell type labels used in th
Wu et al. scRNA-seq datasets

| Major cell types | Minor cell types | | |
|---|---|---|---|
| provided by the authors | provided by the authors | Cell subsets provided by the authors | Cell labels used in this work |
| B-cells | B cells Memory | B cells Memory | B cells |
| B-cells | B cells Naive | B cells Naive | B cells |
| Myeloid | DCs | Myeloid_c11_cDC2_CD1C | Dendritic cells |
| Myeloid | DCs | Myeloid_c4_DCs_pDC_IRF7 | Dendritic cells |
| Myeloid | DCs | Myeloid_c3_cDC1_CLEC9A | Dendritic cells |
| Myeloid | DCs | Myeloid_c0_DC_LAMP3 | Dendritic cells |
| Endothelial | Endothelial ACKR1 | Endothelial ACKR1 | Endothelial cells |
| Endothelial | Endothelial RGS5 | Endothelial RGS5 | Endothelial cells |
| Endothelial | Endothelial CXCL12 | Endothelial CXCL12 | Endothelial cells |
| Endothelial | Endothelial Lymphatic LYVE1 | Endothelial Lymphatic LYVE1 | Endothelial cells |
| Normal Epithelial | Myoepithelial | Myoepithelial | Epithelial cells |
| Normal Epithelial | Luminal Progenitors | Luminal Progenitors | Epithelial cells |
| Normal Epithelial | Mature Luminal | Mature Luminal | Epithelial cells |
| Cancer Epithelial | Cancer Cycling | Cancer Cycling | Epithelial cells |
| Cancer Epithelial | Cancer Her2 SC | Cancer Her2 SC | Epithelial cells |
| Cancer Epithelial | Cancer LumB SC | Cancer LumB SC | Epithelial cells |
| Cancer Epithelial | Cancer Basal SC | Cancer Basal SC | Epithelial cells |
| Cancer Epithelial | Cancer LumA SC | Cancer LumA SC | Epithelial cells |
| CAFs | CAFs MSC iCAF-like | CAFs MSC iCAF-like s1 | Fibroblasts |
| CAFs | CAFs MSC iCAF-like | CAFs MSC iCAF-like s2 | Fibroblasts |
| CAFs | CAFs myCAF-like | CAFs Transitioning s3 | Fibroblasts |
| CAFs | CAFs myCAF-like | CAFs myCAF like s4 | Fibroblasts |
| CAFs | CAFs myCAF-like | CAFs myCAF like s5 | Fibroblasts |
| Myeloid | Macrophage | Myeloid_c10_Macrophage_1_EGR1 | Monocytes and Macrophages |
| Myeloid | Monocyte | Myeloid_c12_Monocyte_1_IL1B | Monocytes and Macrophages |
| Myeloid | Macrophage | Myeloid_c2_LAM2_APOE | Monocytes and Macrophages |
| Myeloid | Macrophage | Myeloid_c1_LAM1_FABP5 | Monocytes and Macrophages |
| Myeloid | Monocyte | Myeloid_c8_Monocyte_2_S100A9 | Monocytes and Macrophages |
| Myeloid | Monocyte | Myeloid_c7_Monocyte_3_FCGR3A | Monocytes and Macrophages |
| Myeloid | Macrophage | Myeloid_c9_Macrophage_2_CXCL10 | Monocytes and Macrophages |
| Myeloid | Macrophage | Myeloid_c5_Macrophage_3_SIGLEC1 | Monocytes and Macrophages |
| T-cells | NK cells | T_cells_c9_NK_cells_AREG | NK cells |
| T-cells | NKT cells | T_cells_c10_NKT_cells_FCGR3A | NK cells |
| Plasmablasts | Plasmablasts | Plasmablasts | PCs |
| PVL | PVL Differentiated | PVL Differentiated s3 | PVL |
| PVL | PVL Immature | PVL_Immature s2 | PVL |
| PVL | PVL Immature | PVL Immature s1 | PVL |
| PVL | Cycling PVL | Cycling PVL | PVL |
| T-cells | T cells CD4+ | T_cells_c0_CD4+_CCR7 | T cells CD4 |
| T-cells | T cells CD4+ | T_cells_c1_CD4+_IL7R | T cells CD4 |
| T-cells | T cells CD4+ | T_cells_c2_CD4+_T-regs_FOXP3 | T cells CD4 |
| T-cells | T cells CD4+ | T_cells_c3_CD4+_Tfh_CXCL13 | T cells CD4 |
| T-cells | T cells CD8+ | T_cells_c4_CD8+_ZFP36 | T cells CD8 |
| T-cells | T cells CD8+ | T_cells_c6_IFIT1 | T cells CD8 |
| T-cells | T cells CD8+ | T_cells_c5_CD8+_GZMK | T cells CD8 |
| T-cells | T cells CD8+ | T_cells_c7_CD8+_IFNG | T cells CD8 |
| T-cells | T cells CD8+ | T_cells_c8_CD8+_LAG3 | T cells CD8 |
| T-cells | Cycling T-cells | T_cells_c11_MKI67 | Excluded |
| Myeloid | Cycling_Myeloid | Cycling_Myeloid | Excluded |

Lee et al. scRNA-seq dataset

| Major cell types | Cell subsets | |
|---|---|---|
| provided by the authors | provided by the authors | Cell labels used in this work |
| B cells | CD19+CD20+ B | B cells |
| B cells | IgA+ Plasma | Plasma cells |
| B cells | lgG+ Plasma | Plasma cells |
| T cells | CD4+ T cells | T cells CD4 |
| T cells | Regulatory T cells | T cells CD4 |
| T cells | T follicular helper cells | T cells CD4 |
| T cells | T helper 17 cells | T cells CD4 |
| T cells | CD8+ T cells | T cells CD8 |
| T cells | NK cells | NK cells |
| Myeloids | Pro-inflammatory | Monocytes and Macrophages |
| Myeloids | Proliferating | Monocytes and Macrophages |
| Myeloids | SPP1+ | Monocytes and Macrophages |
| Myeloids | cDC | Dendritic cells |
| Mast cells | Mast cells | Mast cells |
| Stromal cells | Myofibroblasts | Fibroblasts |
| Stromal cells | Stromal 1 | Fibroblasts |
| Stromal cells | Stromal 2 | Fibroblasts |
| Stromal cells | Stromal 3 | Fibroblasts |
| Stromal cells | Lymphatic ECs | Endothelial cells |
| Stromal cells | Proliferative ECs | Endothelial cells |
| Stromal cells | Stalk-like ECs | Endothelial cells |
| Stromal cells | Tip-like ECs | Endothelial cells |
| Epithelial cells | CMS1 | Epithelial cells |
| Epithelial cells | CMS2 | Epithelial cells |
| Epithelial cells | CMS3 | Epithelial cells |
| Epithelial cells | CMS4 | Epithelial cells |
| Stromal cells | Smooth muscle cells | Smooth muscle cells |
| Stromal cells | Pericytes | Pericytes |
| Stromal cells | Enteric glial cells | Enteric glial cells |
| Epithelial cells | Mature Enterocytes type 2 | Excluded |
| Epithelial cells | Goblet cells | Excluded |
| Epithelial cells | Intermediate | Excluded |
| Epithelial cells | Mature Enterocytes type 1 | Excluded |
| Epithelial cells | Stem-like/TA | Excluded |
| T cells | gamma delta T cells | Excluded |
| T cells | Unknown | Excluded |

TABLE 3
Running time analysis.
Comparison of running times (shown in minutes) across methods for representative scRNA-
seq/ST datasets analyzed in this work. In all cases, the core CytoSPACE mapping function
was run with a single CPU, whereas Tangram and CellTrek were each run with 24 CPU cores.
For the fractional abundance inference step via Spatial Seurat, 24 CPU cores were provided.
For all methods, data loading and file writing were excluded from reported running times.
Comparison of running times (shown in minutes) across CytoSPACE solvers and other methods

| | | | Seurat + | Seurat + | | |
|---|---|---|---|---|---|---|
| | CytoSPACE | CytoSPACE | CytoSPACE | CytoSPACE | | |
| | exact | integer | exact | integer | CellTrek | Tangram |
| Dataset | (1 core) | (1 core) | (1 core) | (1 core) | (24 cores) | (24 cores) |
| Melanoma 1 (Tirosh et al.) | 0.7 | 0.5 | 10.2 | 10.0 | 10.7 | 3.6 |
| Melanoma 2 (Tirosh et al.) | 1.0 | 0.8 | 10.3 | 10.1 | 10.0 | 5.4 |
| HER2+ BRCA (Wu et al.) | 5.1 | 2.6 | 8.6 | 6.0 | 19.7 | 64.7 |
| ER+HER2+ BRCA (Wu et al.) | 6.8 | 5.6 | 13.6 | 12.4 | 36.5 | 168.7 |
| TNBC BRCA (Wu et al.) | 24.9 | 9.5 | 45.9 | 30.5 | 142.3 | 268.7 |
| CRC (Lee et al.) | 8.1 | 31.6 | 30.4 | 53.9 | 186.2 | 72.4 |

TABLE 4
Solver comparison.
Concordance between CytoSPACE solvers (exact shortest augmenting path
vs. integer approximation cost scaling push-relabel methods) for single-
cell spot assignment in selected datasets. In all cases tested, greater
than 99% of cells were assigned to the same spot between solver methods.
Overlap of cell-to-spot assignments across
CytoSPACE solvers (exact vs. integer)

| Dataset | Percentage of cells |
|---|---|
| Melanoma 1 (Tirosh et al.) | 100.0 |
| Melanoma 2 (Tirosh et al.) | 100.0 |
| HER2+ BRCA (Wu et al.) | 99.9 |
| ER+HER2+ BRCA (Wu et al.) | 99.5 |
| TNBC BRCA (Wu et al.) | 100.0 |
| CRC (Lee et al.) | 99.9 |

TABLE 5
Data underlying mouse kidney analysis.

| | | | | | [symbol] | |
|---|---|---|---|---|---|---|
| | | | | Mean Euclidean | ordering | Ground truth |
| Cell | | | | distance to state | from state | distance to |
| type/state | | Epithelial | | 32 by CytoSPACE | 19 to state | state 32 |
| number^{a} | Epithelial cell state^{a} | type | Zone | (base of inner medulla) | [symbol] ^{b} | (FIG. 2i)^{c} |
| 19 | Nephron connecting tubule | Nephron | Cortex | 33.10 | 1 | 15 |
| 18 | Distal convoluted tubule | Nephron | Cortex | 30.88 | 2 | 14 |
| 3, 4 | Segment 1 of proximal tubule | Nephron | Cortex | 32.98 | 2 | 14 |
| 17 | Macula densa | Nephron | Cortex | 26.92 | 3 | 13 |
| 1 | Podocytes (visceral epithelium) | Nephron | Cortex | 43.97 | 4 | 12 |
| 2 | Parietal epithelium | Nephron | Cortex | 33.85 | 4 | 12 |
| 5, 6 | Segment 2 of proximal tubule | Nephron | Cortex | 30.80 | 5 | 11 |
| 16 | Distal straight tubule of outer stripe of OM an [symbol] | Nephron | Cortex | 23.11 | 6 | 10 |
| 7, 8 | Segment 3 of proximal tubule | Nephron | Outer stripe | 23.63 | 7 | 9 |
| 9A | LOH thin descending limb of inner stripe of O [symbol] | Nephron | Inner stripe | 18.65 | 8 | 8 |
| 9B | LOH thin descending limb of inner stripe of O [symbol] | Nephron | Inner stripe | 20.89 | 8 | 8 |
| 15 | Distal straight tubule of inner stripe of OM | Nephron | Inner stripe | 16.19 | 9 | 7 |
| 10 | Upper LOH thin descending limb of IM of juxt [symbol] | Nephron | Inner medulla | 18.39 | 10 | 6 |
| 11 | Lower LOH thin descending limb of IM of juxt [symbol] | Nephron | Inner medulla | 17.27 | 12 | 4 |
| 13 | Lower LOH thin limb of IM of juxtamedullary n [symbol] | Nephron | Inner medulla | 7.43 | 14 | 2 |
| 20 | Principal-like cell of nephron connecting tubul [symbol] | Ureteric | Cortex | 33.69 | 2 | 14 |
| 21 | Intercalated type non-A non-B cell of nephron | Ureteric | Cortex | 35.97 | 3 | 13 |
| 22 | Intercalated type A cell of nephron connecting | Ureteric | Cortex | 28.41 | 3 | 13 |
| 23 | Principal-like cell of cortical collecting duct | Ureteric | Cortex | 29.32 | 5 | 11 |
| 24 | Intercalated type B cell of cortical collecting d [symbol] | Ureteric | Cortex | 32.73 | 5 | 11 |
| 25 | Intercalated type A cell of OM collecting duct | Ureteric | Outer stripe | 31.35 | 7 | 9 |
| 26 | Principal cell of OM collecting duct | Ureteric | Inner stripe | 16.01 | 8 | 8 |
| 27 | Intercalated type A cell of IM collecting duct | Ureteric | Inner medulla | 20.07 | 10 | 6 |
| 28 | Principal cell of IM collecting duct type 1 | Ureteric | Inner medulla | 12.65 | 11 | 5 |
| 29 | Principal cell of IM collecting duct type 2 | Ureteric | Inner medulla | 11.00 | 11 | 5 |
| 30 | Principal-like cell of deep IM collecting duct ty [symbol] | Ureteric | Inner medulla | 14.19 | 13 | 3 |
| 31 | Cell of deep IM collecting duct type 2 | Ureteric | Inner medulla | 13.32 | 13 | 3 |
| 32 | Deep medullary epithelium of pelvis | Ureteric | Inner medulla | 8.87 | 15 | 1 |

[symbol]

indicates data missing or illegible when filed

TABLE 6
Data underlying T cell state analysis of MERSCOPE data.
Gene sets for T cell states and corresponding
known tumor enrichment ranks Lower rank indicates
enrichment in adjacent normal tissue; higher rank

| Known | | scRNA-seq | |
|---|---|---|---|
| tumor | | mapped to | |
| enrichment | | MERSCOPE by | MERSCOPE |
| rank | CD4 T cell state | NES | NES |
| 1 | Temra | −1.20 | 0.85 |
| 2 | CREM+ Tm | −1.92 | −1.20 |
| 3 | TNF+ | 0.87 | 0.86 |
| 4 | AREG+ Tm | −2.47 | −1.56 |
| 5 | CCL5+ Tm | −1.96 | −0.89 |
| 6 | Tn | −2.22 | −1.41 |
| 7 | CCR6+ Th17 | −2.03 | −1.18 |
| 8 | ADSL+ Tn | −1.76 | −1.63 |
| 9 | CXCR5+ Tfh | −2.01 | −1.81 |
| 10 | CAPG+CREM− Tm | 0.89 | 1.09 |
| 11 | IL26+ Th17 | −1.39 | −1.02 |
| 12 | TIMP1+ Tm | 0.89 | −1.03 |
| 13 | GZMK+ Tem | 0.84 | 0.96 |
| 14 | IL21+ Tfh | 0.94 | 0.66 |
| 15 | NME1+CCR4+ | 1.57 | −1.29 |
| 16 | CAPG+ Tm | 1.21 | −1.04 |
| 17 | TNFRSF9− Treg | 1.59 | 0.73 |
| 18 | S1PR1+ Treg | 1.20 | −1.23 |
| 19 | NME1+CCR4− | 1.64 | 1.22 |
| 20 | TNFRSF9+ Treg | 1.89 | 1.54 |
| 21 | IFNG+ Th | 1.60 | 1.79 |
| 22 | ISG+ Th | 1.00 | −1.03 |
| 23 | ISG+ Treg | 1.75 | 1.20 |

| Known | | SCRNA-seq | |
|---|---|---|---|
| tumor | | mapped to | |
| enrichment | | MERSCOPE by | MERSCOPE |
| rank | CD8 T cell state | NES | NES |
| 1 | KIR+EOMES+ NK-like | −1.35 | −1.65 |
| 2 | IL7R+ Tm | −2.12 | −1.21 |
| 3 | Temra | 1.16 | 0.76 |
| 4 | Tc17 | −1.51 | −0.95 |
| 5 | KIR+TXK+ NK-like | 0.98 | 1.26 |
| 6 | ZNF683+CXCR6− Tm | 1.55 | 1.02 |
| 7 | Tn | −2.24 | −2.01 |
| 8 | ZNF683+CXCR6+ Trm | 1.79 | 1.13 |
| 9 | GZMK+ early Tem | −1.76 | −1.73 |
| 10 | TCF7+ Tex | 1.09 | −1.50 |
| 11 | GZMK+ Tem | 1.49 | −1.38 |
| 12 | NME1+ | 1.68 | 1.76 |
| 13 | GZMK+ Tex | 1.84 | 1.15 |
| 14 | OXPHOS− Tex | 1.44 | 1.25 |
| 15 | Terminal Tex | 1.98 | 1.91 |
| 16 | ISG+ | 1.41 | 0.70 |

1-13. (canceled)

14. A method for yielding a spatially resolved map of a specimen, using a computational processing system, comprising:
obtaining spatial omics data from a plurality of regions that cover a specimen, wherein the specimen comprises a plurality of cell types;
estimating a number of cells per region from the spatial omics data
and a fraction of each cell type from the spatial omics data
querying referential single cell omics data to match a number of cells for each cell type to yield single cell omics data for spatial assignment; and
based on a globally optimal solution, assigning single cells from the single cell omics data to spatial coordinates to yield a spatially resolved map of the specimen.

15. The method of claim 14, wherein the spatial omics is one of: spatial transcriptomics, spatial genomics, spatial epigenomics, spatial methylomics, spatial proteomics, or spatial metabolomics.

16. The method of claim 14 further comprising: extracting source material to perform the spatial omics from each region of the plurality of regions, wherein the source material is extracted via laser capture microdissection, iterative microdigestion, or in situ capture.

17. The method of claim 14, wherein the spatial omics is spatial transcriptomics, the method further comprising:
determining expression of a plurality of transcripts via in situ hybridization.

18. The method of claim 14, wherein the step of estimating the number of cells per region comprises:
estimating, using the computational processing system, the number of cells per region of the plurality of regions based on an amount of source material derived from each region, as determined by the spatial omics data.

19. The method of claim 14, wherein the step of estimating the number of cells per region comprises:
estimating, using the computational processing system, the number of cells per region via cell segmentation.

20. The method of claim 19, wherein each region of the plurality of regions is examined for segmented nuclei or staining of cell membranes and the estimation of the number of cells is based on the nuclei count or cell membrane count.

21. The method of claim 14, wherein estimating a fraction of each cell type of the plurality of cell types is estimated by a deconvolution method.

22. The method of claim 21, wherein the deconvolution method is determined from the spatial omics data and an a priori defined reference.

23. The method of claim 21, wherein the deconvolution method is determined by: Spatial Seurat, RCTD, SPOTlight, cell2location, or CIBERSORTx.

24. The method of claim 14, wherein querying the referential single cell omics data to match the number of cells for each cell type of the plurality of cell types further comprises:
removing, using the computational processing system, single cell omics data of one or more single cells within the referential single cell omics data when the number of single cells within the referential single cell omics data is greater than the number of cells estimated within the specimen.

25. The method of claim 14, wherein querying the referential single cell omics data to match the number of cells for each cell type of the plurality of cell types further comprises:
adding, using the computational processing system, single cell omics data of one or more single cells within the referential single cell omics data when the number of single cells within the referential single cell omics data is less than the number of cells estimated within the specimen.

26. The method of claim 25, wherein adding single cell omics data of one or more single cells comprises duplicating single cell omics data of the single cell omics data.

27. The method of claim 25, wherein adding single cell omics data of one or more single cells comprises generating single cell omics data representative of the single cell omics data.

28. The method of claim 14, wherein each region comprises a number of subregions equal to with the number of cells estimated for each region.

29. The method of claim 28, wherein assigning single cells from the set of single cell omics data to spatial coordinates comprises:
generating, using the computational processing system, matrix of single cell omics profiles with single cells and a matrix of specimen omics profiles with subregions; and
utilizing, using the computational processing system, the matrices to determine a globally optimal solution.

30. The method of claim 29 further comprises:
determining, using the computational processing system, the globally optimal solution by summation of assignments of singles cells to subregions that minimizes a linear cost function.

31. The method of claim 30, wherein the determining the globally optimal solution further comprises:
solving, using the computational processing system, the globally optimal solution via a shortest augmenting paths-based Jonker-Volgenant algorithm.

32. The method of claim 30, wherein the determining the globally optimal solution comprises:
solving, using the computational processing system, the globally optimal solution via a cost scaling push-relabel method.

33. The method of claim 14, wherein the spatially resolved map of the specimen has a single-cell resolution.

34. A method for yielding a spatially resolved map of a specimen, the method comprising:
obtaining, using a computational processing system, spatial omics data from a plurality of regions that cover a specimen, wherein the specimen is a collection of cells that comprises a plurality of cell types;
estimating, using the computational processing system, a number of cells per region of the plurality of regions; and
based on a globally optimal solution, concurrently:
determining a fraction of each cell type of the plurality of cells;
querying, using the computational processing system, referential single cell omics data to match the number of cells for each cell type of the plurality of cell types to yield a set of single cell omics data for spatial assignment; and
assigning, using the computational processing system, single cells from the set of single cell omics data to spatial coordinates to yield a spatially resolved map of the specimen.

35. The method of claim 34, wherein the spatial omics is one of: spatial transcriptomics, spatial genomics, spatial epigenomics, spatial methylomics, spatial proteomics, or spatial metabolomics.

36. The method of claim 34 further comprising:
extracting source material to perform the spatial omics from each region of the plurality of regions, wherein the source material is extracted via laser capture microdissection, iterative microdigestion, or in situ capture.

37. The method of claim 34, wherein the spatial omics is spatial transcriptomics, the method further comprising:
determining expression of a plurality of transcripts via in situ hybridization.

38. The method of claim 34, wherein the step of estimating the number of cells per region comprises:
estimating, using the computational processing system, the number of cells per region of the plurality of regions based on an amount of source material derived from each region, as determined by the spatial omics data.

39. The method of claim 34, wherein the step of estimating the number of cells per region comprises:
estimating, using the computational processing system, the number of cells per region via cell segmentation.

40. The method of claim 39, wherein each region of the plurality of regions is examined for segmented nuclei or staining of cell membranes and the estimation of the number of cells is based on the nuclei count or cell membrane count.

41. The method of claim 34, wherein querying the referential single cell omics data to match the number of cells for each cell type of the plurality of cell types further comprises:
removing, using the computational processing system, single cell omics data of one or more single cells within the referential single cell omics data when the number of single cells within the referential single cell omics data is greater than the number of cells estimated within the specimen.

42. The method of claim 34, wherein querying the referential single cell omics data to match the number of cells for each cell type of the plurality of cell types further comprises: adding, using the computational processing system, single cell omics data of one or more single cells within the referential single cell omics data when the number of single cells within the referential single cell omics data is less than the number of cells estimated within the specimen.

43. The method of claim 42, wherein adding single cell omics data of one or more single cells comprises duplicating single cell omics data of the single cell omics data.

44. The method of claim 42, wherein adding single cell omics data of one or more single cells comprises generating single cell omics data representative of the single cell omics data.

45. The method of claim 34, wherein each region comprises a number of subregions equal to with the number of cells estimated for each region.

46. The method of claim 45, wherein assigning single cells from the set of single cell omics data to spatial coordinates comprises:
generating, using the computational processing system, matrix of single cell omics profiles with single cells and a matrix of specimen omics profiles with subregions; and
utilizing, using the computational processing system, the matrices to determine a globally optimal solution.

47. The method of claim 46 further comprises:
determining, using the computational processing system, the globally optimal solution by summation of assignments of singles cells to subregions that minimizes a linear cost function.

48. The method of claim 47, wherein the determining the globally optimal solution further comprises:
solving, using the computational processing system, the globally optimal solution via a shortest augmenting paths-based Jonker-Volgenant algorithm.

49. The method of claim 47, wherein the determining the globally optimal solution further comprises:
solving, using the computational processing system, the globally optimal solution via a cost scaling push-relabel method.

50. The method of claim 34, wherein the spatially resolved map of the specimen has a single-cell resolution.

51. A method for yielding a spatially resolved map of a specimen for a set of one or more cell types, the method comprising:
obtaining, using a computational processing system, spatial omics data from a plurality of regions that cover a specimen, wherein the specimen is a collection of cells that comprises a plurality of cell types;
estimating, using the computational processing system, a number of cells per region of the plurality of regions from the spatial omics data;
estimating, for each region of the plurality regions, using the computational processing system, a fraction of each cell type of the plurality of cell types from the spatial omics data;
querying, for each region comprising a fraction of a cell type of the set of one or more cell types, using the computational processing system, referential single cell omics data to match the number of cells for each cell type of the plurality of cell types to yield a set of single cell omics data for spatial assignment; and
based on a globally optimal solution, assigning, for each region comprising a fraction of a cell type of the set of one or more cell types, using the computational processing system, single cells from the set of single cell omics data to spatial coordinates to yield a spatially resolved map of the specimen consisting of the cell types of the set of one or more cell types.

52. The method of claim 51, wherein the spatial omics is one of: spatial transcriptomics, spatial genomics, spatial epigenomics, spatial methylomics, spatial proteomics, or spatial metabolomics.

53. The method of claim 51 further comprising:
extracting source material to perform the spatial omics from each region of the plurality of regions, wherein the source material is extracted via laser capture microdissection, iterative microdigestion, or in situ capture.

54. The method of claim 51, wherein the spatial omics is spatial transcriptomics, the method further comprising:
determining expression of a plurality of transcripts via in situ hybridization.

55. The method of claim 51, wherein the step of estimating the number of cells per region comprises:
estimating, using the computational processing system, the number of cells per region of the plurality of regions based on an amount of source material derived from each region, as determined by the spatial omics data.

56. The method of claim 51, wherein the step of estimating the number of cells per region comprises:
estimating, using the computational processing system, the number of cells per region via cell segmentation.

57. The method of claim 56, wherein each region of the plurality of regions is examined for segmented nuclei or staining of cell membranes and the estimation of the number of cells is based on the nuclei count or cell membrane count.

58. The method of claim 51, wherein estimating a fraction of each cell type of the plurality of cell types is estimated by a deconvolution method.

59. The method of claim 58, wherein the deconvolution method is determined from the spatial omics data and an a priori defined reference.

60. The method of claim 58, wherein the deconvolution method is determined by: Spatial Seurat, RCTD, SPOTlight, cell2location, or CIBERSORTx.

61. The method of claim 51, wherein querying the referential single cell omics data to match the number of cells for each cell type of the plurality of cell types further comprises:
removing, for each region comprising a fraction of a cell type of the set of one or more cell types, using the computational processing system, single cell omics data of one or more single cells within the referential single cell omics data when the number of single cells within the referential single cell omics data is greater than the number of cells estimated within the specimen.

62. The method of claim 51, wherein querying the referential single cell omics data to match the number of cells for each cell type of the plurality of cell types further comprises:
adding, for each region comprising a fraction of a cell type of the set of one or more cell types, using the computational processing system, single cell omics data of one or more single cells within the referential single cell omics data when the number of single cells within the referential single cell omics data is less than the number of cells estimated within the specimen.

63. The method of claim 62, wherein adding single cell omics data of one or more single cells comprises duplicating single cell omics data of the single cell omics data.

64. The method of claim 62, wherein adding single cell omics data of one or more single cells comprises generating single cell omics data representative of the single cell omics data.

65. The method of claim 51, wherein each region comprises a number of subregions equal to with the number of cells estimated for each region.

66. The method of claim 65, wherein assigning single cells from the set of single cell omics data to spatial coordinates comprises:
generating, for each region comprising a fraction of a cell type of the set of one or more cell types, using the computational processing system, a matrix of single cell omics profiles with single cells and a matrix of specimen omics profiles with subregions; and
utilizing, using the computational processing system, the matrices to determine a globally optimal solution.

67. The method of claim 66 further comprises:
determining, using the computational processing system, the globally optimal solution by summation of assignments of singles cells to subregions that minimizes a linear cost function.

68. The method of claim 67, wherein the determining the globally optimal solution further comprises:
solving, using the computational processing system, the globally optimal solution via a shortest augmenting paths-based Jonker-Volgenant algorithm.

69. The method of claim 67, wherein the determining the globally optimal solution further comprises:
solving, using the computational processing system, the globally optimal solution via a cost scaling push-relabel method.

70. The method of claim 51, wherein the spatially resolved map of the specimen has a single-cell resolution.

71. A method for yielding a spatially resolved map of a specimen via spatial transcriptomics, the method comprising:
obtaining, using a computational processing system, spatial transcriptomics data from a plurality of regions that cover a specimen, wherein the specimen is a collection of cells that comprises a plurality of cell types;
estimating, using the computational processing system, a number of cells per region of the plurality of regions from the spatial transcriptomics data;
estimating, using the computational processing system, a fraction of each cell type of the plurality of cell types from the spatial transcriptomics data;
querying, using the computational processing system, referential single cell transcriptomics data to match the number of cells for each cell type of the plurality of cell types to yield a set of single cell transcriptomics data for spatial assignment; and
based on a globally optimal solution, assigning, using the computational processing system, single cells from the set of single cell transcriptomics data to spatial coordinates to yield a spatially resolved map of the specimen.

72. The method of claim 71 further comprising: extracting RNA from each region of the plurality of regions, wherein the RNA is extracted via in situ capture; and
sequencing the extracted RNA from each region of the plurality of regions to yield the spatial transcriptomics data from the plurality of regions.

73. The method of claim 72, wherein the RNA is extracted from each region of the plurality of regions via 10xGenomics Visium or NanoString GeoMX.

74. The method of claim 72, wherein the sequencing is performed by one of the following techniques: whole exome sequencing, capture targeted sequencing, amplification-based targeted sequencing, sequencing based on random priming, or end-biased sequencing.

75. The method of claim 71 further comprising:
determining expression of a plurality of transcripts via in situ hybridization to yield the spatial transcriptomics data from the plurality of regions.

76. The method of claim 75, wherein the expression of the plurality of transcripts is determined via Vizgen MERSCOPE, NanoString CosMX, 10×Genomics Xenium, or hybridization-based in situ sequencing.

77. The method of claim 71, wherein the step of estimating the number of cells per region comprises:
estimating, using the computational processing system, the number of cells per region of the plurality of regions based on a number of detectably expressed genes.

78. The method of claim 77, wherein the number of detectably expressed genes is determined by a number of unique molecular identifiers.

79. The method of claim 71, wherein the step of estimating the number of cells per region comprises:
estimating, using the computational processing system, the number of cells per region via cell segmentation.

80. The method of claim 79, wherein each region of the plurality of regions is examined for segmented nuclei or staining of cell membranes and the estimation of the number of cells is based on the nuclei count or cell membrane count.

81. The method of claim 71, wherein estimating a fraction of each cell type of the plurality of cell types is estimated by a deconvolution method.

82. The method of claim 81, wherein the deconvolution method is determined from the spatial transcriptomics data and an a priori defined reference.

83. The method of claim 81, wherein the deconvolution method is determined by: Spatial Seurat, RCTD, SPOTlight, cell2location, or CIBERSORTx.

84. The method of claim 71, wherein querying the referential single cell transcriptomics data to match the number of cells for each cell type of the plurality of cell types further comprises:
removing, using the computational processing system, single cell transcriptomics data of one or more single cells within the referential single cell transcriptomics data when the number of single cells within the referential single cell transcriptomics data is greater than the number of cells estimated within the specimen.

85. The method of claim 71, wherein querying the referential single cell transcriptomics data to match the number of cells for each cell type of the plurality of cell types further comprises:
adding, using the computational processing system, single cell transcriptomics data of one or more single cells within the referential single cell transcriptomics data when the number of single cells within the referential single cell transcriptomics data is less than the number of cells estimated within the specimen.

86. The method of claim 85, wherein adding single cell transcriptomics data of one or more single cells comprises duplicating single cell transcriptomics data of the single cell transcriptomics data.

87. The method of claim 85, wherein adding single cell transcriptomics data of one or more single cells comprises generating single cell transcriptomics data representative of the single cell transcriptomics data.

88. The method of claim 71, wherein each region comprises a number of subregions equal to with the number of cells estimated for each region.

89. The method of claim 88, wherein assigning single cells from the set of single cell transcriptomics data to spatial coordinates comprises:
generating, using the computational processing system, matrix of single cell transcriptomics profiles with single cells and a matrix of specimen transcriptomics profiles with subregions; and
utilizing, using the computational processing system, the matrices to determine a globally optimal solution.

90. The method of claim 89 further comprises:
determining, using the computational processing system, the globally optimal solution by summation of assignments of singles cells to subregions that minimizes a linear cost function.

91. The method of claim 90, wherein the determining the globally optimal solution further comprises:
solving, using the computational processing system, the globally optimal solution via a shortest augmenting paths-based Jonker-Volgenant algorithm.

92. The method of claim 90, wherein the determining the globally optimal solution further comprises:
solving, using the computational processing system, the globally optimal solution via a cost scaling push-relabel method.

93. The method of claim 71, wherein the spatially resolved map of the specimen has a single cell resolution.

94. A method to diagnose a medical disorder based on spatial signatures, comprising:
rendering a spatially resolved map of a tissue specimen extracted from a patient, wherein the rendering a spatially resolved map comprises:
generating spatial omics data from a plurality of regions that cover the tissue specimen;
querying, using a computational processing system, referential single cell omics data to yield a set of single cell omics data for spatial assignment; and
based on a globally optimal solution, assigning, using the computational processing system, single cells from the set of single cell omics data to spatial coordinates to yield the spatially resolved map of the tissue specimen;
assessing the spatially resolved map to detect a presence of a spatial signature, wherein the spatial signature is associated with a characteristic of a medical disorder; and determining the patient has the characteristic of the medical disorder by the
presence of the spatial signature within the spatially resolved map.

95. The method of claim 94, wherein assessing the spatially resolved map to detect the presence of the spatial signature further comprises:
utilizing the rendered spatially resolved map of the tissue specimen as input in a trained machine learning model to yield a likelihood of the characteristic of medical disorder, wherein determining the patient has the characteristic of medical disorder is determined by the likelihood of the characteristic of medical disorder.

96. The method of claim 94, wherein the characteristic of medical disorder is a response to therapy.

97. The method of claim 96 further comprising:
administering the therapy based on a presence of the spatial signature.

98. The method of claim 94, wherein the characteristic of medical disorder is a need for a further diagnostic technique to be performed.

99. The method of claim 98 further comprising:
performing the further diagnostic technique based on a presence of the spatial signature.

100. The method of claim 94 further comprising:
performing a spatial omics protocol using the tissue specimen extracted from the patient, wherein the spatial omics protocol is utilized to render the spatially resolved map.

101. The method of claim 100 further comprising:
extracting the tissue specimen from the patient to perform the spatial omics protocol.

102. The method of claim 94, wherein the tissue specimen comprises tissue of a tumor, of a multicellular organ, infiltrated by immune cells, infected with pathogens, interacting with microbiomes.

103. The method of claim 94, wherein the medical disorder is cancer, a pathogenic infection, an organ dysfunction, an inflammatory disorder, an autoimmune disorder, diabetes, liver dysfunction, heart disease, or a neurodegenerative disorder.

104. The method of claim 94, wherein the characteristic of the medical disorder is a particular pathology, a likelihood of success or failure of a therapy, a severity of the medical disorder, a need for a particular medical intervention, or a likelihood of a future medical complication.

105. A method to diagnose a cancer based on spatial signatures, comprising:
rendering a spatially resolved map of a tumor specimen from a patient, wherein the rendering a spatially resolved map comprises:
generating spatial omics data from a plurality of regions that cover the tumor specimen;
querying, using a computational processing system, referential single cell omics data to yield a set of single cell omics data for spatial assignment; and
based on a globally optimal solution, assigning, using the computational processing system, single cells from the set of single cell omics data to spatial coordinates to yield the spatially resolved map of the tumor specimen;
assessing the spatially resolved map to detect a presence of a spatial signature, wherein the spatial signature is associated with a cancer characteristic; and
determining the patient has the cancer characteristic by the presence of the spatial signature within the spatially resolved map.

106. The method of claim 105, wherein assessing the spatially resolved map to detect the presence of the spatial signature further comprises:
utilizing the rendered spatially resolved map of the tumor specimen as input in a trained machine learning model to yield a likelihood of the cancer characteristic; wherein determining the patient has the cancer characteristic of medical disorder is determined by the likelihood of the cancer characteristic.

107. The method of claim 105, wherein the cancer characteristic is a response to a therapy, a toxicity of a therapy, or a resistance to a therapy.

108. The method of claim 107, wherein the cancer characteristic is the response to the therapy, the method further comprising:
administering the therapy based on a presence of the spatial signature, wherein the presence of the spatial signature indicates the patient will respond to the therapy.

109. The method of claim 107, wherein the cancer characteristic is the response to the therapy, the method further comprising:
administering the therapy based on a lack of a presence of the spatial signature, wherein the presence of the spatial signature indicates the patient will not respond to the therapy.

110. The method of claim 107, wherein the cancer characteristic is the toxicity of the therapy, the method further comprising:
administering the therapy based on a presence of the spatial signature, wherein the presence of the spatial signature indicates the therapy is not toxic to the patient.

111. The method of claim 107, wherein the cancer characteristic is the toxicity of the therapy, the method further comprising:
administering the therapy based on a lack of a presence of the spatial signature, wherein the presence of the spatial signature indicates the therapy is toxic to the patient.

112. The method of claim 107, wherein the cancer characteristic is the resistance to the therapy, the method further comprising:
administering the therapy based on a presence of the spatial signature, wherein the presence of the spatial signature indicates the patient will not be resistant to the therapy.

113. The method of claim 107, wherein the cancer characteristic is the resistance to the therapy, the method further comprising:
administering the therapy based on a lack of a presence of the spatial signature, wherein the presence of the spatial signature indicates the patient will be resistant to the therapy.

114. The method of claim 107, wherein the therapy comprises one of: immunotherapy, chemotherapy, radiotherapy, a targeted therapy, hormone therapy, or surgical resection.

115. The method of claim 105 further comprising:
performing a spatial omics protocol using the tumor specimen extracted from the patient, wherein the spatial omics protocol is utilized to render the spatially resolved map.

116. The method of claim 115 further comprising:
extracting the tumor specimen from the patient to perform the spatial omics protocol.

117. The method of claim 105, wherein the cancer characteristic is cancer progression, a likelihood of metastasis, a transition from pre-invasive to invasive cancer, or a likelihood of recurrence.

118. A method for training a machine learning model to predict spatial signatures from spatially resolved maps, comprising:
rendering a spatially resolved map of a plurality of multicellular specimens, wherein each multicellular specimen is associated with a biological characteristic;
rendering a spatially resolved map of a plurality of multicellular control specimens, wherein each multicellular control specimen is not associated with the biological characteristic,
wherein rendering of each spatially resolved map comprises:
generating spatial omics data from a plurality of regions that cover the specimen;
querying, using a computational processing system, referential single cell omics data to yield a set of single cell omics data for spatial assignment;
based on a globally optimal solution, assigning, using the computational processing system, single cells from the set of single cell omics data to spatial coordinates to yield the spatially resolved map of the specimen; and
training a machine learning model with each spatially resolved map of the plurality of multicellular specimens and of the plurality of multicellular control specimens to predict the biological characteristic from a spatially resolved map.

119. The method of claim 118, wherein the biological characteristic comprises a pathology, a medical disorder, a health status, a metabolic status, an organ status, an activation of multicellular communication, a multicellular transition, or a multicellular response to a stimulus.

120. The method of claim 118, wherein each multicellular specimen is a tumor specimen and the biological characteristic is a cancer characteristic selected from: a response to a therapy, a toxicity of a therapy, or a resistance to a therapy.

121. The method of claim 120, wherein the therapy comprises one of: immunotherapy, chemotherapy, radiotherapy, a targeted therapy, hormone therapy, or surgical resection.

122. The method of claim 118, wherein each multicellular specimen is a tumor specimen and the biological characteristic is a cancer characteristic selected from: cancer progression, a likelihood of metastasis, a transition from pre-invasive to invasive cancer, or a likelihood of recurrence.

123. The method of claim 118, wherein the machine learning model is a classifier.

124. The method of claim 118, wherein the machine learning model is a regressor.

125. The method of claim 118, wherein the machine learning model incorporates a deep neural network (DNN), a convolutional neural network (CNN), a graph neural network (GNN), a recurrent neural network, a long short-term memory (LSTM) network, a kernel ridge regression (KRR), or gradient-boosted random forest decision trees.

126. The method of claim 118, wherein the machine learning model incorporates a spatial encoder.
